(19) United States
(12) Patent Application Publication
(10) Pub. No.: US 2014/0227715 A1
(43) Pub. Date: Aug. 14, 2014

(54) Non-Invasive methods to Determine Vulnerable Plaque Burden in Subjects

(71) Applicant: Singulex, Inc., Alameda, CA (US)

(72) Inventors: John Allan Todd, Lafayette, CA (US); Quynh Anh Thuc Lu, Mountain View, CA (US); Jeffrey David Greenberg, Milburn, NJ (US)

(73) Assignee: Singulex, Inc., Alameda, CA (US)

(21) Appl. No.: 14/181,210

(22) Filed: Feb. 14, 2014

Related U.S. Application Data
(60) Provisional application No. 61/764,633, filed on Feb. 14, 2013.

(51) Int. Cl. G01N 33/68

(52) U.S. Cl. CPC G01N 33/6893
USPC 435/7.4; 436/501

(57) ABSTRACT

Methods, kits, and systems for determining vulnerable plaque burden in a subject. The methods involve measuring an amount of at least one biomarker in a blood sample from the subject, wherein the at least one biomarker is selected from the group consisting of vascular endothelial growth factor-A (VEGF), cardiac troponin I (cTnI), and matrix metalloproteinase-9 (MMP-9). The concentration of these biomarkers in a sample from the subject are used to determine vulnerable plaque burden in the subject.

### CROSS REFERENCE TO RELATED APPLICATION

The application claim the benefit of U.S. provisional patent application Ser. No. 61/764,633, filed Feb. 14, 203, which is incorporated by reference herein in its entirety.

### BACKGROUND

Cardiovascular disease (CVD) is an abnormal function of the heart and/or blood vessels. Included under this designation are such diverse medical conditions as coronary artery disease, congestive heart failure, arrhythmia, atherosclerosis, hypertension, stroke, cerebrovascular disease, peripheral vascular disease and myocardial infarction. Worldwide, CVD is a major cause of death with an estimated 17.3 million deaths globally in 2008 attributed to cardiovascular diseases. As of 2011, cardiovascular diseases are the number one cause of death worldwide, representing ˜30% of all global deaths, and CVD is projected to remain the single leading cause of death; projected to account for an estimated 23 million deaths worldwide in 2030.

Cardiovascular disease involves a progressive process with etiologies in both cardiac muscle (cardio-pathology) and vascular inflammation. The disease process follows a continuum from early onset mild vascular inflammation to severe acute events such as acute myocardial infarction or chronic events such as heart failure. Plaque rupture and thrombosis is the main cause for abrupt coronary occlusion leading to acute myocardial infarctions, unstable angina and many cases of sudden cardiac death.

Vulnerable plaques are those that are likely to rupture and are characterized by a thin fibrous cap (≦65 μm thick), a lipid core occupying at 30-40% of the plaque volume (composed of free cholesterol crystals, cholesterol esters, oxidized lipids impregnated with tissue factor), numerous lipid-filled macrophages near the necrotic core and in the cap, abundant lymphocytes in the adventitia, and other inflammatory cells (such as mast cells) in the lesion, as well as adventitial inflammation and neovascularization (formation of new blood vessels in the plaque). The two major determinants of plaque rupture appear to be the size of the lipid-rich core and the thickness of fibrous cap (Thim et al., 2008 J Intern. Med. 263(5): 506-16).

A challenge facing clinicians who have patients presenting with vulnerable plaque risk factors is understanding their degree of risk associated with plaque rupture. Measurement of ¹⁸F-fluorodeoxyglucose (¹⁸FDG) uptake using Positron Emission Tomography/Computed Tomography (PET/CT) is strongly associated with plaque macrophage content as a marker of carotid vascular inflammation and can be used to assess vulnerable plaque burden. These procedures can be time consuming, inconvenient and expensive. Therefore, the inventors have identified a need in the art for highly sensitive and less invasive methods to predict a patient's vulnerable plaque burden.

### SUMMARY

In one aspect, the disclosure is directed to a method for determining vulnerable plaque burden in a subject. The method includes (a) measuring an amount of at least one biomarker in a blood sample from the subject, wherein the at least one biomarker is selected from the group consisting of vascular endothelial growth factor-A (VEGF), cardiac troponin I (cTnI), and matrix metalloproteinase-9 (MMP-9), (b) comparing the amount of the at least one biomarker in the biological sample from the subject to a threshold concentration representing the concentration of the at least one biomarker in a population of normal healthy control subjects, and (c) determining vulnerable plaque burden in the subject when the concentration of at least one in the sample is greater than the threshold concentration.

In another aspect, the disclosure is directed to a method for determining vulnerable plaque burden in a subject. The method includes measuring the concentration of one or more biomarkers selected from the group consisting of VEGF, cTnI and MMP-9 in a blood sample from a patient, and standardizing the biomarker concentrations in the sample to threshold concentrations for the biomarkers in a population of normal healthy controls to create a ratio of the concentrations in the sample and the threshold concentrations. The ratios represent the vulnerable plaque burden in the subject.

In yet another aspect the disclosure is directed to a method for determining vulnerable plaque burden in a subject. The method includes (a) measuring an amount of at least two biomarkers selected from VEGF, cTnI or MMP-9 in a blood sample from a patient, (b) standardizing the biomarker concentrations in the sample to threshold concentrations of the biomarkers in a population of normal healthy controls to create ratios of the concentrations in the test sample and the threshold concentrations, (c) adding the ratios to create a score representing vulnerable plaque burden in the subject.

Optionally in the various methods of the disclosure, measuring the amount of VEGF, cTnI or MMP-9 includes contacting the biological sample with an antibody specific for the VEGF, cTnI and MMP-9, and determining the amount of specific binding between the antibody and the VEGF, cTnI and MMP-9 in the sample.

The methods may further include the use of a highly sensitive single molecule detector for the detection of the concentration of labels representing the concentration of cTnI, MMP-9 and VEGF in patient sample.

### BRIEF DESCRIPTION OF THE FIGURES

FIGS. 1A-1F are graphs comparing the serum levels of VEGF, cTnI and MMP9 alone and in various combinations to vascular inflammation scored by a PET target-to-background signal.

### DESCRIPTION

The use of a biomarker that can determine the vulnerable plaque burden in at risk individuals can facilitate the identification of patients that will have an increased risk of developing cardiovascular disease (CVD). This is important for both patient management as well as drug development. Of particular interest are patients with Rheumatoid Arthritis (RA), who have a two to three fold greater risk of CVD than the general population. The use of biomarkers to identify vulnerable plaque burden can provide for earlier diagnosis of disease and identify patients at higher risk for disease progression.

Unless otherwise defined, the technical and scientific terms used herein have the same meaning as commonly understood by one of ordinary skill in the art. Expansion and clarification of some terms are provided herein. All publications, patent applications, patents and other references mentioned herein, if not otherwise indicated, are explicitly incorporated by reference.

As used herein, the singular forms “a,” “an”, and “the” include plural referents unless the context clearly dictates otherwise.

As used herein, the term “subject” refers to a mammal, typically a human, that can be afflicted by CVD, but may or may not have such a disease. The terms “subject” and “patient” are used herein interchangeably in reference.

As used herein, the term “sample” is taken broadly to include any sample suitable for the methods described herein. Typically, the sample is a biological sample such as, for example, a biological fluid such as blood, which includes serum and/or plasma, samples.

As used herein, the term “healthy volunteer control subject average concentrations” or “healthy control average concentrations” refers to the average concentration of the various biomarkers described herein for at least two subjects who do not have a vulnerable plaque burden (e.g., healthy volunteers, or control subjects). Preferably, average concentration values are calculated from biomarker concentrations measured in larger groups. Healthy control average concentrations are provided herein, but one of skill in the art may also measure biomarker concentrations in one or more populations of subjects lacking vulnerable plaques utilizing an apparatus capable of sensitively measuring the concentrations of biomarkers described herein and calculating the average values for each biomarker in such control subject populations.

As used herein, the term “therapy” refers to the administration of any medical treatment (e.g., pharmaceuticals) or interventional treatment (e.g., surgery) to affect vulnerable plaque or the biomarkers relevant to vulnerable plaque described herein.

As used herein, the term “CV” refers to the coefficient of variance. As used herein, the term “average CV” refers to average of the coefficient of variance obtained for all samples tested in triplicate.

The term “antibody,” as used herein, refers to naturally occurring antibodies as well as non-naturally occurring antibodies, including, for example, single chain antibodies, chimeric, bifunctional and humanized antibodies, as well as antigen-binding fragments thereof. It will be appreciated that the choice of epitope or region of the molecule to which the antibody is raised will determine its specificity, e.g., for various forms of the molecule, if present, or for total (e.g., all, or substantially all, of the molecule). In various embodiments, the antibody is a polyclonal antibody or a monoclonal antibody.

Antibodies to VEGF, cTnI or MMP-9 are well characterized in the field of the disclosure. Antibodies to VEGF, cTnI or MMP-9 are available from a variety of commercial and non-commercial sources. The disclosure is not limited to any of the particular antibodies described for exemplary purposes.

In one aspect the disclosure is directed to the use of plasma proteomics of growth factors, troponins and matrix metalloproteinases that can serve as individual, additive or combinatorial biomarkers for determining vulnerable plaque burden in at risk patients.

Vascular Endothelial Growth Factor-A, commonly known as VEGF, can be used as a biomarker for determining vulnerable plaque burden. VEGF is a member of a family of secreted glycoproteins that promote endothelial cell growth, survival, migration, and vascular permeability, all of which contribute to angiogenesis. The binding of VEGF to its receptor triggers the activation of a cell signaling pathway that is critical for the growth of blood vessels from pre-existing vasculature. VEGF is implicated in a variety of diseases including cancer, age-related macular degeneration, diabetic retinopathy and RA.

Cardiac troponin is another protein of interest as a biomarker for determining vulnerable plaque burden in at risk patients. Cardiac troponin is a marker of cardiac damage, and is useful in the diagnosis and prognosis of a number of diseases and conditions (e.g., acute myocardial infarct (AMI)). Because the level of troponin found in the circulation of healthy individuals is very low, and cardiac specific troponins do not arise from extra-cardiac sources, the troponins are very sensitive and specific markers of cardiac injury. See U.S. Pat. No. 7,838,250, which is incorporated by reference in its entirety.

Another set of proteins that are of interest as biomarkers for determining vulnerable plaque burden in at risk patients are the matrix metalloproteinases. Certain studies have shown that some of the matrix metalloproteinases (MMP) are molecular mediators of inflammation and that cells found in artheroscelrotic plaques express MMP's. The 92-kD collagenase, or MMP-9, is produced by normal alveolar macrophages and granulocytes. MMP-9 is also known as 92-kD gelatinase, type V collagenase, and gelatinase-9. MMP-9 is present in most cases of inflammatory responses. Most MMP-9 is secreted as an inactive proprotein which are activated when cleaved by extracellular proteinases. Either proMMP-9 or total-MMP (tMMP-9) are useful biomarkers in the detection of vulnerable plaque.

cTnI, VEGF, and MMP-9 are associated with vulnerable plaque as measured by radiographic findings in at risk patients. According to the one aspect of the disclosure, differences in growth factors, troponins and matrix metalloproteinase concentrations can be determined between various patients with highly sensitive assays. Measuring these concentrations in a patient sample allows for determining vulnerable plaque burden in at risk patients.

In some embodiments, the present disclosure provides methods for quantifying normal levels of VEGF, cTnI or MMP-9, and identifying abnormally elevated levels of VEGF, cTnI or MMP-9, which is indicative of the presence of vascular inflammation or vulnerable plaque burden in a subject. This measurement can be augmented by co-measurement of the same or other biomarkers in healthy control individuals, where elevated levels are indicative of inflammation.

In one aspect, the disclosure provides methods for detecting an amount of VEGF, cTnI or MMP-9, in a biological sample from a patient. The amount of VEGF, cTnI or MMP-9 in a patient's biological sample can be correlated to the carotid vascular inflammation in an at risk patient.

In order to determine the vulnerable plaque burden in at risk patients, the concentration of at least one VEGF, cTnI and MMP-9 is measured in a biological sample from the subject. Measurement can be accomplished with several known methods, including the highly sensitive methods described herein. Once the marker(s) has been measured, its concentration can be compared to the amount of the marker(s) in the sample to a threshold concentration representing the concentration of the marker(s) in a population of normal healthy control subjects. Thresholds can be set using a variety of methods, including but not limited to analyses of biomarker concentrations using samples obtained from healthy control populations. Using these populations, biomarker concentrations are determined for each sample in the population and then averages, medians, or 75th percentiles are calculated and then used as threshold values. For instance, in plasma from a population of normal healthy control populations, the average levels of VEGF, cTnI and MMP-9 were found to be 19.61 pg/mL, 3.17 pg/mL and 179 ng/mL, respectively; and hence these values would represent thresholds based upon average determinations. Vulnerable plaque burden in the subject is determined when the concentration of at least one biomarker in the sample is greater than the threshold concentration.

In another embodiment, the threshold concentration may be reported in the literature or may be another suitable value as determined by one of skill in the art.

Also, the threshold concentration can be calculated from the concentration of VEGF, cTnI or MMP-9 or other growth factors, troponins or metalloproteinases in biological control samples taken from the same patient at earlier time points or healthy volunteer control subjects.

In another embodiment, the disclosure involves standardizing the biomarker concentrations in the biological test sample to a threshold concentration of the biomarkers in a population of normal healthy controls to create a ratio of the concentration in the test sample and the threshold concentrations. For example, for cTnI, ratios of at least 2 (patient cTnI concentration/healthy control cTnI concentration) is indicative of vulnerable plaque burden in the patient. In other embodiment, the ratio is at least 3, at least 4, at least 5, at least 6, at least 7, at least 8, at least 9, at least 10, at least 11, at least 12, at least 15, at least 20, at least 25, at least 30, at least 50, at least 75 and at least 100. Similarly, Patient/Control MMP-9 or VEGF concentration ratios indicative of vulnerable plaque may be at least 2, at least 3, at least 4, at least 5, at least 6, at least 7, at least 8, at least 9, at least 10, at least 11, at least 12, at least 15, at least 20, at least 25, at least 30, at least 50, at least 75 and at least 100. In some embodiments, the reference sample can be from the same patient, but taken at an earlier time point

In yet another embodiment, the disclosure includes creating a score of the vulnerable plaque burden in a subject. For example, the score can be obtained by determining the ratios of the concentrations of the biomarkers in the test sample and the threshold concentrations, and adding the ratios to create a score representing vulnerable plaque burden in the subject. Accordingly, the patient marker concentration/control population marker concentration ratio for at least two of cTnI, MMP-9 and VEGF are added to create a score. For instance, as shown in the Examples below, when the cTnI ratio is 14.2, the VEGF ratio is 6.7, the total MMP-9 ratio is 12.0 and the pro-MMP ration is 99.1, the score may be 20.9 (cTnI+VEGF), 32.9 (cTnI+VEGF+TMMP-9) or 120 (cTnI+VEGF+pMMP-9). Other similar scores can be calculated with these four markers. The score can be indicative of vulnerable plaque (for example, as determined by measurement of a carotid Maximum Disease Segment (MDS) as is known in the art).

In another embodiment, the additive score (e.g., cTnI+VEGF+MMP-9) can be normalized to provide a normalized score on a particular scale, such as a scale of 1-100, thereby providing a caregiver with a useful tool when considering the extent of vulnerable plaque.

In another embodiment, the scale is from 1 to 10, or uses any appropriate scale that is useful to the caregiver.

Detection and Characterization of Vulnerable Plaque

Imaging plaque formation in carotid arteries can be used a surrogate for coronary artery plaque, and this load is can be measured using a Multimodal Magnetic Resonance Imaging (MRI). Multimodal MRI enables imaging of plaque morphology (e.g. plaque geometry, volume, area, or wall thickness) and plaque composition, which reflects the characteristics of plaque vulnerability, such as a necrotic core, haemorrhage, calcification or fibrous cap thickness. Using Dynamic Contrast Enhanced (DCE) MRI enables imaging of plaque neovascularization, which is a hallmark of plaque inflammation and vulnerability.

Additionally, imaging plaques can be performed using fluorodeoxyglucose (FDG) uptake measured using positron emission tomography/computerized tomography (PET/CT). Noninvasive imaging in PET and computerized tomography angiography (CTA) can be applied to detecting vulnerable plaque. Recently, it has been shown that ¹⁸F-fluorodeoxyglucose (¹⁸FDG) uptake in plaques, in both animal models and human patients, is proportional to the degree of inflammation and macrophage density, and this can be used to identify and localize inflamed plaques and thrombosis (Aziz et al., Circulation, 117:2061-70 (2008)). ¹⁸FDG-PET imaging can be used to assess the severity of inflammation in carotid plaques in patients (Tawakol et al., J Am Coll Cardiol., 48(9):1818-24 (2006)). Furthermore, uptake of ¹⁸FDG on PET imaging may indicate a higher risk of cardiovascular events (Paulmier et al., J Nucl Cardiol., 15(2):209-17 (2008)). The measurement of ¹⁸FDG uptake using PET/CT is strongly associated with plaque macrophage content as a marker of carotid vascular inflammation and can be used to assess vulnerable plaque burden. A PET target-to-background ratio (TBR) score for carotid vascular inflammation in patients can be generated based on their radiographs and can be associated with the presence or amount of biomarkers in a patient sample.

In one aspect, the disclosure is directed to methods of determining the vulnerable plaque burden of a patient comprising: measuring an amount of VEGF, cTnI or MMP-9 in a first biological test sample from a patient; measuring an amount of the VEGF, cTnI or MMP-9 in control sample(s) from a healthy volunteer subject(s) to determine a threshold value based upon the average value; comparing the amount of the VEGF, cTnI or MMP-9 in the test sample to the threshold values for VEGF, cTnI or MMP-9, and determining that a patient has an increased risk of vulnerable plaque burden by determining that the amount of the VEGF, cTnI or MMP-9 in the first sample is greater than the threshold values.

In another aspect, biomarkers in a patient sample are detected and compared to ¹⁸FDG uptake using ¹⁸FDG-PET imaging of, for example, the left or right common carotid arteries, descending aorta or ascending aorta. ¹⁸FDG uptake, as indicated by a larger target-to-background ratio, reflects a greater degree of vascular inflammation. Vascular inflammation as detected by ¹⁸FDG uptake can be compared to the amount of biomarker in the patient sample and accepted statistical analysis (e.g., student's t-test or Spearman correlation coefficient) can be used to establish a relationship.

The degree of vulnerable plaque in a target area or subject can be given a score by standardizing the biomarker concentrations from patients samples to threshold levels determined in a population of healthy control volunteers. A score could be given based on a single biomarker or by combining one, two, three or more biomarkers. The score can represent, for example, the target area or subject's vulnerable plaque burden and can be ranked from, for example, 1-100, where 1 represents the least amount of vulnerable plaque and 100 represents the most vulnerable plaque. A higher score is consistent with a greater degree of ¹⁸FDG uptake as determined by the target-to-background ratio from PET imaging for the target area. A target area can be, for example, the maximal diseased segment of the left and/or right common carotid artery, descending aorta and/or ascending aorta. A target area can also be the mean target-to-background ratio (TBR) for a given target area, for example, the left and/or right common carotid arteries, descending aorta and/or ascending aorta.

Elevated levels of VEGF, cTnI or MMP-9 are consistent with vulnerable plaque burden and/or carotid vascular inflammation. These observations confirm that VEGF, cTnI or MMP-9 are prognostic biomarkers for vulnerable plaque burden and/or carotid vascular inflammation. As one example, the ratios of the concentrations of each of VEGF, cTnI and MMP-9 in a patient blood sample (e.g., serum) and a threshold concentration representing the concentration in normal healthy control samples can be added to create a specific score of vulnerable plaque burden.

Instruments and Systems Suitable for Highly Sensitive Analysis Biomarkers

In one aspect, the disclosure provides a method for determining the presence or absence of a single molecule, e.g., a molecule of a marker of a biological state, in a sample, by i) labeling the molecule if present, with a label; and ii) detecting the presence or absence of the label, where the detection of the presence of the label indicates the presence of the single molecule in the sample. In some embodiments, the method is capable of detecting the molecule at a limit of detection of less than about 100, 80, 60, 50, 40, 30, 20, 15, 12, 10, 9, 8, 7, 6, 5, 4, 3, 2, 1, 0.9, 0.8, 0.7, 0.6, 0.5, 0.4, 0.3, 0.2, 0.1, 0.05, 0.01, 0.005, or 0.001 femtomolar. Detection limits may be determined by use of an appropriate standard, e.g., National Institute of Standards and Technology reference standard material.

The methods also provide methods of determining a concentration of a molecule, e.g., a marker indicative of a biological state, in a sample by detecting single molecules of the molecule in the sample. The “detecting” of a single molecule includes detecting the molecule directly or indirectly. In the case of indirect detection, labels that correspond to single molecules, e.g., labels attached to the single molecules, can be detected.

In some embodiments, the disclosure provides a method for determining the presence or absence of a single molecule of a protein in a biological sample, comprising labeling said molecule with a label and detecting the presence or absence of said label in a single molecule detector, wherein said label includes a fluorescent moiety that is capable of emitting at least about 200 photons when simulated by a laser emitting light at the excitation wavelength of the moiety, wherein the laser is focused on a spot not less than about 5 microns in diameter that contains the moiety, and wherein the total energy directed at the spot by the laser is no more than about 15 microJoules. The single molecule detector may, in some embodiments, include not more than one interrogation space. The limit of detection of the single molecule in the sample may be less than about 10, 1, 0.1, 0.01, or 0.001 femtomolar. In some embodiments, the limit of detection is less than about 1 femtomolar. The detecting may include detecting electromagnetic radiation emitted by said fluorescent moiety. The method may further include exposing said fluorescent moiety to electromagnetic radiation, e.g., electromagnetic radiation provided by a laser, such as a laser with a power output of about 2, 3, 4, 5, 6, 7, 8, 9, 10, 11, 12, 13, 14, 15, 16, 17, 18, 19, 20 mW. In some embodiments, the laser stimulus provides light to the interrogation space for between about 10-1000 microseconds, or about 1000, 250, 100, 50, 25 or 10 microseconds. In some embodiments, the label further includes a binding partner specific for binding a target molecule, such as an antibody. In some embodiments, the fluorescent moiety includes a fluorescent dye molecule, such as a dye molecule that includes at least one substituted indolium ring system in which the substituent on the 3-carbon of the indolium ring contains a chemically reactive group or a conjugated substance. The method may further include measuring the concentration of said protein in the sample.

In some embodiments, detecting the presence or absence of said label includes: (i) passing a portion of said sample through an interrogation space; (ii) subjecting said interrogation space to exposure to electromagnetic radiation, said electromagnetic radiation being sufficient to stimulate said fluorescent moiety to emit photons, if said label is present; and (iii) detecting photons emitted during said exposure of step (ii). The method may further include determining a background photon level in said interrogation space, wherein said background level represents the average photon emission of the interrogation space when it is subjected to electromagnetic radiation in the same manner as in step (ii), but without label in the interrogation space. The method may further include comparing the amount of photons detected in step (iii) to a threshold photon level, wherein said threshold photon level is a function of said background photon level, wherein an amount of photons detected in step (iii) greater that the threshold level indicates the presence of said label, and an amount of photons detected in step (iii) equal to or less than the threshold level indicates the absence of said label.

Labels

One skilled in the art will recognize that many strategies can be used for labeling target molecules to enable their detection or discrimination in a mixture of molecules. The labels may be attached by any known means, including methods that utilize non-specific or specific interactions of label and target. Labels may provide a detectable signal or affect the mobility of the target molecule in an electric field. In addition, labeling can be accomplished directly or through binding partners.

In some embodiments, the label includes a binding partner to the molecule of interest, where the binding partner is attached to a fluorescent moiety. The compositions and methods of the disclosure may utilize highly fluorescent moieties, e.g., a moiety capable of emitting at least about 200 photons when simulated by a laser emitting light at the excitation wavelength of the moiety, wherein the laser is focused on a spot not less than about 5 microns in diameter that contains the moiety, and wherein the total energy directed at the spot by the laser is no more than about 15 microJoules, for example, no more than about 12 microJoules, no more than about 9 microJoules, no more than about 6 microJoules, and no more than about 3 microJoules. Moieties suitable for the compositions and methods of the disclosure are described in more detail below.

In some embodiments, a label for detecting a biological molecule includes a binding partner for the biological molecule that is attached to a fluorescent moiety. In some embodiments, the moiety includes a plurality of fluorescent entities, e.g., about 2 to 4, 2 to 5, 2 to 6, 2 to 7, 2 to 8, 2 to 9, 2 to 10, or about 3 to 5, 3 to 6, 3 to 7, 3 to 8, 3 to 9, or 3 to 10 fluorescent entities. The fluorescent entities can be fluorescent dye molecules. In some embodiments, the fluorescent dye molecules include at least one substituted indolium ring system in which the substituent on the 3-carbon of the indolium ring contains a chemically reactive group or a conjugated substance. In some embodiments, the dye molecules are ALEXA FLUOR® dye molecules (Life Technologies), such as for example, ALEXA FLUOR® 488, ALEXA FLUOR® 532, ALEXA FLUOR® 647, ALEXA FLUOR® 680 or ALEXA FLUOR® 700, ALEXA FLUOR® 750. Other examples of suitable fluorescent molecules include fluorescein, B-phycoerythrin, allophycocyanin, PBXL-3, and Qdot® 605. In some embodiments, the dye molecules include a first type and a second type of dye molecule, e.g., two different ALEXA FLUOR® molecules, where the first type and second type of dye molecules have different emission spectra. The ratio of the number of first type to second type of dye molecule can be, e.g., 4 to 1, 3 to 1, 2 to 1, 1 to 1, 1 to 2, 1 to 3 or 1 to 4.

Binding Partners

Any suitable binding partner with the requisite specificity for the form of target molecule to be detected may be used. If the molecule, e.g., a marker, has several different forms, various specificities of binding partners are possible. Suitable binding partners are known in the art and include antibodies, aptamers, lectins, and receptors. A useful and versatile type of binding partner is an antibody.

In some embodiments, the binding partner is an antibody specific for a molecule to be detected. The term “antibody,” as used herein, is a broad term and is used in its ordinary sense, including, without limitation, to refer to naturally occurring antibodies as well as non-naturally occurring antibodies, including, for example, single chain antibodies, chimeric, bifunctional and humanized antibodies, as well as antigen-binding fragments thereof. It will be appreciated that the choice of epitope or region of the molecule to which the antibody is raised will determine its specificity, e.g., for various forms of the molecule, if present, or for total (e.g., all, or substantially all, of the molecule).

In some embodiments, the antibody is a polyclonal antibody. In other embodiments, the antibody is a monoclonal antibody. Methods for producing antibodies are well-established. One skilled in the art will recognize that many procedures are available for the production of antibodies, for example, as described in Antibodies, A Laboratory Manual, Ed Harlow and David Lane, Cold Spring Harbor Laboratory (1988). One skilled in the art will also appreciate that binding fragments or Fab fragments which mimic antibodies can also be prepared from genetic information by various procedures (Antibody Engineering: A Practical Approach (Borrebaeck, C., ed.), 1995, Oxford University Press; J. Immunol. 149, 3914-3920 (1992)). Monoclonal and polyclonal antibodies also commercially available from a variety of commercial sources. (e.g., R and D Systems, Minneapolis, Minn.; HyTest Ltd., Turku Finland; Abcam Inc., Cambridge, Mass.; Life Diagnostics, Inc., West Chester, Pa.; Fitzgerald Industries International, Inc., Concord, Mass.; BiosPacific, Emeryville, Calif.).

Capture binding partners and detection binding partner pairs, e.g., capture and detection antibody pairs may be used in embodiments of the disclosure. Thus, in some embodiments, a heterogeneous assay protocol is used in which, typically, two binding partners, e.g., two antibodies, are used. One binding partner is a capture partner, usually immobilized on a solid support, and the other binding partner is a detection binding partner, typically with a detectable label attached. Antibody pairs can be designed and prepared by methods well known in the art. Compositions of the disclosure include antibody pairs wherein one member of the antibody pair is a label as described herein, and the other member is a capture antibody.

Fluorescent Moieties

In some embodiments of labels used in the disclosure, the binding partner, e.g., antibody, is attached to a fluorescent moiety. The fluorescence of the moiety will be sufficient to allow detection in a single molecule detector, such as the single molecule detectors described herein.

A “fluorescent moiety,” as that term is used herein, includes one or more fluorescent entities whose total fluorescence is such that the moiety may be detected in the single molecule detectors described herein. Thus, a fluorescent moiety may include a single entity (e.g., a Quantum Dot or fluorescent molecule) or a plurality of entities (e.g., a plurality of fluorescent molecules). It will be appreciated that when “moiety,” as that term is used herein, refers to a group of fluorescent entities, e.g., a plurality of fluorescent dye molecules, each individual entity may be attached to the binding partner separately or the entities may be attached together, as long as the entities as a group provide sufficient fluorescence to be detected.

Typically, the fluorescence of the moiety involves a combination of quantum efficiency and lack of photobleaching sufficient that the moiety is detectable above background levels in a single molecule detector, with the consistency necessary for the desired limit of detection, accuracy, and precision of the assay. For example, in some embodiments, the fluorescence of the fluorescent moiety is such that it allows detection and/or quantitation of a molecule, e.g., a marker, at a limit of detection of less than about 10, 9, 8, 7, 6, 5, 4, 3, 2, 1, 0.1, 0.01, 0.001, 0.00001, or 0.000001 pg/ml and with a coefficient of variation of less than about 20, 15, 14, 13, 12, 11, 10, 9, 8, 7, 6, 5, 4, 3, 2, 1%, in the instruments described herein or other instruments that provide suitable quantitative accuracy.

“Limit of detection,” or LoD, as those terms are used herein, includes the lowest concentration at which one can identify a sample as containing a molecule of the substance of interest, e.g., the first non-zero value. It can be defined by the variability of zeros and the slope of the standard curve. For example, the limit of detection of an assay may be determined by running a standard curve, determining the standard curve zero value, and adding 2 standard deviations to that value. A concentration of the substance of interest that produces a signal equal to this value is the “lower limit of detection” concentration.

Furthermore, a fluorescent moiety has properties that are consistent with its use in the assay of choice. In some embodiments, the assay is an immunoassay, where the fluorescent moiety is attached to an antibody; the moiety must have properties such that it does not aggregate with other antibodies or proteins, or experiences no more aggregation than is consistent with the required accuracy and precision of the assay. In some embodiments, fluorescent moieties that are preferred are fluorescent moieties, e.g., dye molecules that have a combination of 1) high absorption coefficient; 2) high quantum yield; 3) high photostability (low photobleaching); and 4) compatibility with labeling the molecule of interest (e.g., protein) so that it may be analyzed using the analyzers and systems, for example, as described herein (e.g., does not cause precipitation of the protein of interest, or precipitation of a protein to which the moiety has been attached).

Fluorescent moieties, e.g., a single fluorescent dye molecule or a plurality of fluorescent dye molecules, that are useful in some embodiments of the disclosure may be defined in terms of their photon emission characteristics when stimulated by EM radiation. For example, in some embodiments, the disclosure utilizes a fluorescent moiety, e.g., a moiety comprising a single fluorescent dye molecule or a plurality of fluorescent dye molecules, that is capable of emitting an average of at least about 10, 20, 30, 40, 50, 75, 100, 125, 150, 175, 200, 225, 250, 275, 300, 350, 400, 500, 600, 700, 800, 900, or 1000, photons when simulated by a laser emitting light at the excitation wavelength of the moiety, where the laser is focused on a spot of not less than about 5 microns in diameter that contains the moiety, and where the total energy directed at the spot by the laser is no more than about 15 microJoules. It will be appreciated that the total energy may be achieved by many different combinations of power output of the laser and length of time of exposure of the dye moiety. For example, in order to acheive total energy of 15 microJoules, a laser of a power output of 1 mW may be used for 15 ms, 3 mW for 5 ms, and so on.

In some embodiments, the disclosure utilizes a fluorescent dye moiety, e.g., a single fluorescent dye molecule or a plurality of fluorescent dye molecules, that is capable of emitting an average of at least about 200 photons when simulated by a laser emitting light at the excitation wavelength of the moiety, where the laser is focused on a spot of not less than about 5 microns in diameter that contains the moiety, and wherein the total energy directed at the spot by the laser is no more than about 15 microJoules.

In some embodiments, the fluorescent moiety includes an average of at least about 1, 2, 3, 4, 5, 6, 7, 8, 9, or 10 fluorescent entities, e.g., fluorescent molecules. In some embodiments, the fluorescent moiety includes an average of no more than about 2, 3, 4, 5, 6, 7, 8, 9, 10, or 11 fluorescent entities, e.g., fluorescent molecules. In some embodiments, the fluorescent moiety includes an average of about 1 to 11, or about 2 to 10, or about 2 to 8, or about 2 to 6, or about 2 to 5, or about 2 to 4, or about 3 to 10, or about 3 to 8, or about 3 to 6, or about 3 to 5, or about 4 to 10, or about 4 to 8, or about 4 to 6, or about 2, 3, 4, 5, 6, or more than about 6 fluorescent entities. By “average” it is meant that, in a given sample that is representative of a group of labels of the disclosure, where the sample contains a plurality of the binding partner-fluorescent moiety units, the molar ratio of the particular fluorescent entity to the binding partner, as determined by standard analytical methods, corresponds to the number or range of numbers specified. For example, in embodiments wherein the label includes a binding partner that is an antibody and a fluorescent moiety that includes a plurality of fluorescent dye molecules of a specific absorbance, a spectrophotometric assay can be used in which a solution of the label is diluted to an appropriate level and the absorbance at 280 nm is taken to determine the molarity of the protein (antibody) and an absorbance at, e.g., 650 nm (for ALEXA FLUOR® 647), is taken to determine the molarity of the fluorescent dye molecule. The ratio of the latter molarity to the former represents the average number of fluorescent entities (dye molecules) in the fluorescent moiety attached to each antibody.

Dyes

A non-exlusive list of useful fluorescent entities for use in the fluorescent moieties of the disclosure is given in Table 2 of U.S. Patent Publication No. 2009/0234202, which includes a number of ALEXA FLUOR® dyes, Atto dyes (Attec-tec GmbH, Germany), and Dyomic Fluors (Dyomics GmbH, Germany).

Other suitable dyes for use in the disclosure include modified carbocyanine dyes. One such modification includes modification of an indolium ring of the carbocyanine dye to permit a reactive group or conjugated substance at the number three position. The modification of the indolium ring provides dye conjugates that are uniformly and substantially more fluorescent on proteins, nucleic acids and other biopolymers, than conjugates labeled with structurally similar carbocyanine dyes bound through the nitrogen atom at the number one position. In addition to having more intense fluorescence emission than structurally similar dyes at virtually identical wavelengths, and decreased artifacts in their absorption spectra upon conjugation to biopolymers, the modified carbocyanine dyes have greater photostability and higher absorbance (extinction coefficients) at the wavelengths of peak absorbance than the structurally similar dyes. Thus, the modified carbocyanine dyes result in greater sensitivity in assays using the modified dyes and their conjugates. Preferred modified dyes include compounds that have at least one substituted indolium ring system in which the substituent on the 3-carbon of the indolium ring contains a chemically reactive group or a conjugated substance. Other dye compounds include compounds that incorporate an azabenzazolium ring moiety and at least one sulfonate moiety. The modified carbocyanine dyes that can be used to detect individual molecules in various embodiments of the disclosure are described in U.S. Pat. No. 6,977,305, which is herein incorporated by reference in its entirety. Thus, in some embodiments the labels of the disclosure utilize a fluorescent dye that includes a substituted indolium ring system in which the substituent on the 3-carbon of the indolium ring contains a chemically reactive group or a conjugated substance group.

The ALEXA FLUOR® dyes are disclosed in U.S. Pat. Nos. 6,977,305; 6,974,874; 6,130,101; and 6,974,305 which are herein incorporated by reference in their entirety. Some embodiments of the disclosure utilize a dye chosen from the group consisting of ALEXA FLUOR® 647, ALEXA FLUOR® 488, ALEXA FLUOR® 532, ALEXA FLUOR® 555, ALEXA FLUOR® 610, ALEXA FLUOR® 680, ALEXA FLUOR® 700, and ALEXA FLUOR® 750. Some embodiments of the disclosure utilize the ALEXA FLUOR® 647 molecule, which has an absorption maximum between about 650 and 660 nm and an emission maximum between about 660 and 670 nm. The ALEXA FLUOR® 647 dye is used alone or in combination with other Alexa Fluor dyes.

Currently available organic fluors can be improved by rendering them less hydrophobic by adding hydrophilic groups such as polyethylene. Alternatively, currently sulfonated organic fluors such as the ALEXA FLUOR® 647 dye can be rendered less acidic by making them zwitterionic. Particles such as antibodies that are labeled with the modified fluors are less likely to bind non-specifically to surfaces and proteins in immunoassays, and thus enable assays that have greater sensitivity and lower backgrounds. Methods for modifying and improving the properties of fluorescent dyes for the purpose of increasing the sensitivity of a system that detects single molecules are known in the art. Preferably, the modification improves the Stokes shift while maintaining a high quantum yield.

Quantum Dots

In some embodiments, the fluorescent label moiety that is used to detect a molecule in a sample using the analyzer systems of the disclosure is a quantum dot. Quantum dots (QDs), also known as semiconductor nanocrystals or artificial atoms, are semiconductor crystals that contain anywhere between 100 to 1,000 electrons and range from 2-10 nm. Some QDs can be between 10-20 nm in diameter. QDs have high quantum yields, which makes them particularly useful for optical applications. QDs are fluorophores that fluoresce by forming excitons, which are similar to the excited state of traditional fluorophores, but have much longer lifetimes of up to 200 nanoseconds. This property provides QDs with low photobleaching. The energy level of QDs can be controlled by changing the size and shape of the QD, and the depth of the QDs' potential. One optical feature of small excitonic QDs is coloration, which is determined by the size of the dot. The larger the dot, the redder, or more towards the red end of the spectrum the fluorescence. The smaller the dot, the bluer or more towards the blue end it is. The bandgap energy that determines the energy and hence the color of the fluoresced light is inversely proportional to the square of the size of the QD. Larger QDs have more energy levels which are more closely spaced, thus allowing the QD to absorb photons containing less energy, i.e., those closer to the red end of the spectrum. Because the emission frequency of a dot is dependent on the bandgap, it is possible to control the output wavelength of a dot with extreme precision. In some embodiments the protein that is detected with the single molecule analyzer system is labeled with a QD. In some embodiments, the single molecule analyzer is used to detect a protein labeled with one QD and using a filter to allow for the detection of different proteins at different wavelengths.

QDs have broad excitation and narrow emission properties which, when used with color filtering, require only a single electromagnetic source to resolve individual signals during multiplex analysis of multiple targets in a single sample. Thus, in some embodiments, the analyzer system includes one continuous wave laser and particles that are each labeled with one QD. Colloidally prepared QDs are free floating and can be attached to a variety of molecules via metal coordinating functional groups. These groups include but are not limited to thiol, amine, nitrile, phosphine, phosphine oxide, phosphonic acid, carboxylic acids or other ligands. By bonding appropriate molecules to the surface, the quantum dots can be dispersed or dissolved in nearly any solvent or incorporated into a variety of inorganic and organic films. Quantum dots (QDs) can be coupled to streptavidin directly through a maleimide ester coupling reaction or to antibodies through a meleimide-thiol coupling reaction. This yields a material with a biomolecule covalently attached on the surface, which produces conjugates with high specific activity. In some embodiments, the protein that is detected with the single molecule analyzer is labeled with one quantum dot. In some embodiments, the quantum dot is between 10 and 20 nm in diameter. In other embodiments, the quantum dot is between 2 and 10 nm in diameter. In other embodiments, the quantum dot is about 2 nm, 3 nm, 4 nm, 5 nm, 6 nm, 7 nm, 8 nm, 9 nm, 10 nm, 11 nm, 12 nm, 13 nm, 14 nm, 15 v, 16 nm, 17 nm, 18 nm, 19 nm or 20 nm in diameter. Quantum Dots useful in the disclosure include Qdot® 525, Qdot® 565, Qdot® 585, Qdot® 605, Qdot® 655, Qdot® 705, and Qdot® 800 (Life Technologies, Inc.).

Binding Partner-Fluorescent Moiety Compositions

The labels of the disclosure generally contain a binding partner, e.g., antibody, bound to a fluorescent moiety to provide the requisite fluorescence for detection and quantitation in analytical instruments, for example single molecule detectors. Any suitable combination of binding partner and fluorescent moiety for detection in the single molecule detectors described herein may be used as label. The antibody may be any antibody as described above. A fluorescent moiety may be attached such that the label is capable of emitting an average of at least about 50, 75, 100, 125, 150, 175, 200, 225, 250, 275, 300, 350, 400, 500, 600, 700, 800, 900, or 1000, photons when simulated by a laser emitting light at the excitation wavelength of the moiety, where the laser is focused on a spot of not less than about 5 microns in diameter that contains the label, and wherein the total energy directed at the spot by the laser is no more than about 15 microJoules.

Attachment of the fluorescent moiety, or fluorescent entities that make up the fluorescent moiety, to the binding partner, e.g., antibody, may be by any suitable means; such methods are well-known in the art and exemplary methods are given in the Examples. In some embodiments, after attachment of the fluorescent moiety to the binding partner to form a label for use in the methods of the disclosure, and prior to the use of the label for labeling the protein of interest, it is useful to perform a filtration step. E.g., an antibody-dye label may be filtered prior to use, e.g., through a 0.2 micron filter, or any suitable filter for removing aggregates. Other reagents for use in the assays of the disclosure may also be filtered, e.g., through a 0.2 micron filter, or any suitable filter. Without being bound by theory, it is thought that such filtration removes a portion of the aggregates of the, e.g., antibody-dye labels. As such aggregates can bind as a unit to the protein of interest, but upon release in elution buffer are likely to disaggregate, false positives may result; i.e., several labels will be detected from an aggregate that has bound to only a single protein molecule of interest. Regardless of theory, filtration has been found to reduce false positives in the subsequent assay and to improve accuracy and precision.

It will be appreciated that immunoassays often employ a sandwich format, in which binding partner pairs, e.g., antibodies, to the same molecule, e.g., a marker, are used. The disclosure also encompasses binding partner pairs, e.g., antibodies, wherein both antibodies are specific to the same molecule, e.g., the same marker, and wherein at least one member of the pair is a label as described herein. Thus, for any label that includes a binding-partner and a fluorescent moiety, the disclosure also encompasses a pair of binding partners wherein the first binding partner, e.g., antibody, is part of the label, and the second binding partner, e.g., antibody, is, typically, unlabeled and serves as a capture binding partner. In addition, binding partner pairs are frequently used in FRET assays. FRET assays useful in the disclosure are disclosed in U.S. Patent Publication No. 2006/0078998, incorporated by reference herein in its entirety, and the present disclosure also encompasses binding partner pairs, each of which includes a FRET label.

Sample

The sample may be any suitable sample. Typically, the sample is a biological sample, e.g., a biological fluid. Such fluids include, without limitation, bronchoalveolar lavage fluid (BAL), blood, serum, plasma, urine, nasal swab, cerebrospinal fluid, pleural fluid, synovial fluid, peritoneal fluid, amniotic fluid, gastric fluid, lymph fluid, interstitial fluid, tissue homogenate, cell extracts, saliva, sputum, stool, physiological secretions, tears, mucus, sweat, milk, semen, seminal fluid, vaginal secretions, fluid from ulcers and other surface eruptions, blisters, and abscesses, and extracts of tissues including biopsies of normal, malignant, and suspect tissues or any other constituents of the body which may contain the target particle of interest. Other similar specimens such as cell or tissue culture or culture broth are also of interest.

In some embodiments, the sample is a blood sample. In some embodiments the sample is a plasma sample. In some embodiments the sample is a serum sample.

In general, any method of sample preparation may be used that produces a label corresponding to a molecule of interest, e.g., a marker of a biological state to be measured, where the label is detectable in analytical instruments, such as for example the single molecule detector as described herein. As is known in the art, sample preparation in which a label is added to one or more molecules may be performed in a homogeneous or heterogeneous format. In some embodiments, the sample preparation is formed in a homogenous format. In analyzer systems employing a homogenous format, unbound label is not removed from the sample. See, e.g., U.S. Patent Publication No. 2006/0078998.

In some embodiments, the particle or particles of interest are labeled by addition of labeled antibody or antibodies that bind to the particle or particles of interest.

In some embodiments, a heterogeneous assay format is used, where, typically, a step is employed for removing unbound label. Such assay formats are well-known in the art. One particularly useful assay format is a sandwich assay, e.g., a sandwich immunoassay. In this format, the molecule of interest, e.g., marker of a biological state, is captured, e.g., on a solid support, using a capture binding partner. Unwanted molecules and other substances may then optionally be washed away, followed by binding of a label comprising a detection binding partner and a detectable label, e.g., fluorescent moiety. Further washes remove unbound label, then the detectable label is released, usually though not necessarily still attached to the detection binding partner. In alternative embodiments, sample and label are added to the capture binding partner without a wash in between, e.g., at the same time. Other variations will be apparent to one of skill in the art.

In some embodiments, the method for detecting the molecule of interest, e.g., marker of a biological state, uses a sandwich assay with antibodies, e.g., monoclonal antibodies as capture binding partners. The method includes binding molecules in a sample to a capture antibody that is immobilized on a binding surface, and binding the label comprising a detection antibody to the molecule to form a “sandwich” complex. The label includes the detection antibody and a fluorescent moiety, as described herein, which is detected, e.g., using the single molecule analyzers of the disclosure. Both the capture and detection antibodies specifically bind the molecule. Many examples of sandwich immunoassays are known, and some are described in U.S. Pat. No. 4,168,146 to Grubb et al. and U.S. Pat. No. 4,366,241 to Tom et al., both of which are incorporated herein by reference. Further examples specific to specific markers are described in the Examples.

The capture binding partner may be attached to a solid support, e.g., a microtiter plate or paramagnetic beads. In some embodiments, the disclosure provides a binding partner for a molecule of interest, e.g., marker of a biological state, attached to a paramagnetic bead. Any suitable binding partner that is specific for the molecule that it is wished to capture may be used. The binding partner may be an antibody, e.g., a monoclonal antibody. It will be appreciated that antibodies identified herein as useful as a capture antibody may also be useful as detection antibodies, and vice versa.

The attachment of the binding partner, e.g., antibody, to the solid support may be covalent or noncovalent. In some embodiments, the attachment is noncovalent. An example of a noncovalent attachment well-known in the art is biotin-avidin/streptavidin interactions. Thus, in some embodiments, a solid support, e.g., a microtiter plate or a paramagnetic bead, is attached to the capture binding partner, e.g., antibody, through noncovalent attachment, e.g., biotin-avidin/streptavidin interactions. In some embodiments, the attachment is covalent. Thus, in some embodiments, a solid support, e.g., a microtiter plate or a paramagnetic bead, is attached to the capture binding partner, e.g., antibody, through covalent attachment.

The capture antibody can be covalently attached in an orientation that optimizes the capture of the molecule of interest. For example, in some embodiments, a binding partner, e.g., an antibody, is attached in a orientated manner to a solid support, e.g., a microtiter plate or a paramagnetic microparticle.

In some embodiments, the solid support is a microtiter plate. In some embodiments, the solid support is a paramagnetic bead. An exemplary paramagnetic bead is Streptavidin C1 (Dynal, 650.01-03). Other suitable beads will be apparent to those of skill in the art. Methods for attachment of antibodies to paramagnetic beads are well-known in the art.

The molecule of interest is contacted with the capture binding partner, e.g., capture antibody immobilized on a solid support. Some sample preparation may be used; e.g., preparation of serum from blood samples or concentration procedures before the sample is contacted with the capture antibody. Protocols for binding of proteins in immunoassays are well-known in the art and are included in the Examples.

The time allowed for binding will vary depending on the conditions; it will be apparent that shorter binding times are desirable in some settings, especially in a clinical setting. The use of, e.g., paramagnetic beads can reduce the time required for binding. In some embodiments, the time allowed for binding of the molecule of interest to the capture binding partner, e.g., an antibody, is less that about 12, 10, 8, 6, 4, 3, 2, or 1 hours, or less than about 60, 50, 40, 30, 25, 20, 15, 10, or 5 minutes.

In some embodiments, following the binding of particles of the molecule of interest to the capture binding partner, e.g., a capture antibody, particles that bound nonspecifically, as well as other unwanted substances in the sample, are washed away leaving substantially only specifically bound particles of the molecule of interest. In other embodiments, no wash is used between additions of sample and label, which can reduce sample preparation time. Thus, in some embodiments, the time allowed for both binding of the molecule of interest to the capture binding partner, e.g., an antibody, and binding of the label to the molecule of interest, is less that about 12, 10, 8, 6, 4, 3, 2, or 1 hours, or less than about 60, 50, 40, 30, 25, 20, 15, 10, or 5 minutes.

Label is added either with or following the addition of sample and washing. Protocols for binding antibodies and other immunolabels to proteins and other molecules are well-known in the art. If the label binding step is separate from that of capture binding, the time allowed for label binding can be important, e.g., in clinical applications or other time sensitive settings. In some embodiments, the time allowed for binding of the molecule of interest to the label, e.g., an antibody-dye, is less than about 12, 10, 8, 6, 4, 3, 2, or 1 hours, or less than about 60, 50, 40, 30, 25, 20, 15, 10, or 5 minutes. Excess label is removed by washing.

In some embodiments, the label is not eluted from the protein of interest. In other embodiments, the label is eluted from the protein of interest. Preferred elution buffers are effective in releasing the label without generating significant background. It is useful if the elution buffer is bacteriostatic. Elution buffers used in the disclosure can include a chaotrope, a buffer, an albumin to coat the surface of the microtiter plate, and a surfactant selected so as to produce a relatively low background. The chaotrope can include urea, a guanidinium compound, or other useful chaotropes. The buffer can include borate buffered saline, or other useful buffers. The protein carrier can include, e.g., an albumin, such as human, bovine, or fish albumin, an IgG, or other useful carriers. The surfactant can include an ionic or nonionic detergent including Tween 20, Triton X-100, sodium dodecyl sulfate (SDS), and others.

In another embodiment, the solid phase binding assay can be a competitive binding assay. One such method is as follows. First, a capture antibody immobilized on a binding surface is competitively bound by i) a molecule of interest, e.g., marker of a biological state, in a sample, and ii) a labeled analog of the molecule comprising a detectable label (the detection reagent). Second, the amount of the label using a single molecule analyzer is measured. Another such method is as follows. First, an antibody having a detectable label (the detection reagent) is competitively bound to i) a molecule of interest, e.g., marker of a biological state in a sample, and ii) an analog of the molecule that is immobilized on a binding surface (the capture reagent). Second, the amount of the label using a single molecule analyzer is measured. An “analog of a molecule” refers, herein, to a species that competes with a molecule for binding to a capture antibody. Examples of competitive immunoassays are disclosed in U.S. Pat. No. 4,235,601 to Deutsch et al., U.S. Pat. No. 4,442,204 to Liotta, and U.S. Pat. No. 5,208,535 to Buechler et al., all of which are incorporated herein by reference.

Detection of Molecule of Interest and Determination of Concentration

Following elution, the label is run through a single molecule detector in, e.g., the elution buffer. A processing sample may contain no label, a single label, or a plurality of labels. The number of labels corresponds or is proportional to (if dilutions or fractions of samples are used) the number of molecules of the molecule of interest, e.g., marker of a biological state captured during the capture step.

Any suitable single molecule detector capable of detecting the label used with the molecule of interest may be used. Examples, of suitable single molecule detectors are described herein. Typically the detector will be part of a system that includes an automatic sampler for sampling prepared samples, and, optionally, a recovery system to recover samples.

In some embodiments, the processing sample is analyzed in a single molecule analyzer that utilizes a capillary flow system, and that includes a capillary flow cell, a laser to illuminate an interrogation space in the capillary through which processing sample is passed, a detector to detect radiation emitted from the interrogation space, and a source of motive force to move a processing sample through the interrogation space. In some embodiments, the single molecule analyzer further includes a microscope objective lens that collects light emitted from processing sample as it passes through the interrogation space, e.g., a high numerical aperture microscope objective. In some embodiments, the laser and detector are in a confocal arrangement. In some embodiments, the laser is a continuous wave laser. In some embodiments, the detector is an avalanche photodiode detector. In some embodiments, the source of motive force is a pump to provide pressure. In some embodiments, the disclosure provides an analyzer system that includes a sampling system capable of automatically sampling a plurality of samples providing a fluid communication between a sample container and the interrogation space.

In some embodiments, the interrogation space has a volume of between about 0.001 and 500 pL, or between about 0.01 pL and 100 pL, or between about 0.01 pL and 10 pL, or between about 0.01 L and 5 pL, or between about 0.01 pL and 0.5 pL. Similarly the interrogation space has a volume between about 0.02 pL and about 300 pL, or between about 0.02 pL and about 50 pL or between about 0.02 pL and about 5 pL or between about 0.02 pL and about 0.5 pL or between about 0.02 pL and about 2 pL, or between about 0.05 pL and about 50 pL, or between about 0.05 pL and about 5 pL, or between about 0.05 pL and about 0.5 pL, or between about 0.05 pL and about 0.2 pL, or between about 0.1 pL and about 25 pL. For example, between about 0.004 pL and 100 pL, between about 0.02 pL and 50 pL, between about 0.001 pL and 10 pL, between about 0.001 pL and 10 pL, between about 0.01 pL and 5 pL, between about 0.02 pL and about 5 pL, between about 0.05 pL and 5 pL, between about 0.05 pL and 10 pL, between about 0.5 pL and about 5 pL, or between about 0.02 pL and about 0.5 pL.

In some embodiments, the interrogation space has a volume of more than about 1 μm³, more than about 2 μm³, for example, more than about 3 μm³, 4 μm³, 5 μm³, 10 μm³, 15 μm³, 30 μm³, 50 μm³, 75 μm³, 100 μm³, 150 μm³, 200 μm³, 250 μm³, 300 μm³, 400 μm³, 500 μm³, 550 μm³, 600 μm³, 750 μm³, 1000 μm³, 2000 μm³, 4000 μm³, 6000 μm³, 8000 μm³, 10000 μm³, 12000 μm³, 13000 μm³, 14000 μm³, 15000 μm³, 20000 μm³, 30000 μm³, 40000 μm³, or more than about 50000 μm³. In some embodiments, the interrogation space is of a volume less than about 50000 μm³, fore example, less than about 40000 μm³, 30000 μm³, 20000 μm³, 15000 μm³, 14000 μm³, 13000 μm³, 12000 μm³, less 11000 μm³, 9500 μm³, 8000 μm³, 6500 μm³, 6000 μm³, 5000 μm³, 4000 μm³, 3000 μm³, 2500 μm³, 2000 μm³, 1500 μm³ 1000 μm³, 800 μm³, 600 μm³, 400 μm³, 200 μm³, 100 μm³, 75 μm³, 50 μm³, 25 μm³, 20 μm³,15 μm³, 14 μm³, 13 μm³, 12 μm³, 11 μm³, 10 μm³, 5 μm³, 4 μm³, μm³, less than about 2 μm³, or less than about 1 μm³. In some embodiments, the volume of the interrogation space is between about 1 μm³ and about 10000 μm³, between about 1 μm³ and about 1000 μm³, between about 1 μm³ and about 100 μm³ between about 1 μm³ and about 50 μm³ between about 1 μm³ and about 10 μm³, 2 μm³ and about 10 μm³ or between about 3 μm³ and about 7 μm³.

In some embodiments, an example of a single molecule detector used in the methods of the disclosure utilizes a capillary flow system, and includes a capillary flow cell, a continuous wave laser to illuminate an interrogation space in the capillary through which processing sample is passed, a high numerical aperture microscope objective lens, for example at least about 0.8, that collects light emitted from processing sample as it passes through the interrogation space, an avalanche photodiode detector to detect radiation emitted from the interrogation space, and a pump to provide pressure to move a processing sample through the interrogation space. In any of these embodiments the analyzer does contain not more than one interrogation space.

In some embodiments, the single molecule detector includes a scanning analyzer system, as disclosed in U.S. Pat. No. 7,914,734, entitled “Scanning Analyzer for Single Molecule Detection and Methods of Use,” which is incorporated by reference herein in its entirety. In some embodiments, the single molecule detector used in the methods of the disclosure uses a sample plate, a continuous wave laser directed toward a sample plate in which the sample is contained, a high numerical aperture microscope objective lens that collects light emitted from the sample as interrogation space is translated through the sample, wherein the lens has a numerical aperture of at least about 0.8, an avalanche photodiode detector to detect radiation emitted from the interrogation space, and a scan motor with a moveable mirror to translate the interrogation space. In any of these embodiments, the analyzer can contain only one interrogation space.

In some embodiments, the single molecule detector is capable of determining a concentration for a molecule of interest in a sample where sample may range in concentration over a range of at least about 100-fold, or 1000-fold, or 10.000-fold, or 100.000-fold, or 300.00-fold, or 1,000,000-fold, or 10,000,000-fold, or 30,000,000-fold.

In some embodiments, the methods of the disclosure utilize a single molecule detector capable detecting a difference of less than about 50%, 40%, 30%, 20%, 15%, or 10% in concentration of an analyte between a first sample and a second sample that are introduced into the detector, where the volume of the first sample and said second sample introduced into the analyzer is less than about 100, 90, 80, 70, 60, 50, 40, 30, 20, 15, 10, 5, 4, 3, 2, or 1 μl, and wherein the analyte is present at a concentration of less than about 100, 90, 80, 70, 60, 50, 40, 30, 20, 15, 10, 5, 4, 3, 2, or 1 femtomolar.

The single molecule detector and systems are described in more detail below. Further embodiments of single molecule analyzers useful in the methods of the disclosure, such as detectors with more than one interrogation window, detectors utilize electrokinetic or electrophoretic flow, and the like, may be found in U.S. Patent Publication No. 2006/0078998, incorporated by reference herein in its entirety.

Between runs the instrument may be washed. A wash buffer that maintains the salt and surfactant concentrations of the sample may be used in some embodiments to maintain the conditioning of the capillary; i.e., to keep the capillary surface relatively constant between samples to reduce variability.

A feature that contributes to the extremely high sensitivity of the instruments and methods of the disclosure is the method of detecting and counting labels, which, in some embodiments, are attached to single molecules to be detected or, more typically, correspond to a single molecule to be detected. Briefly, the processing sample flowing through the capillary or contained on a sample plate is effectively divided into a series of detection events, by subjecting a given interrogation space of the capillary to EM radiation from a laser that emits light at an appropriate excitation wavelength for the fluorescent moiety used in the label for a predetermined period of time, and detecting photons emitted during that time. Each predetermined period of time is a “bin.” If the total number of photons detected in a given bin exceeds a predetermined threshold level, a detection event is registered for that bin, i.e., a label has been detected. If the total number of photons is not at the predetermined threshold level, no detection event is registered. In some embodiments, processing sample concentration is dilute enough that, for a large percentage of detection events, the detection event represents only one label passing through the window, which corresponds to a single molecule of interest in the original sample, that is, few detection events represent more than one label in a single bin. In some embodiments, further refinements are applied to allow greater concentrations of label in the processing sample to be detected accurately, i.e., concentrations at which the probability of two or more labels being detected as a single detection event is no longer insignificant.

Although other bin times can be used without departing from the scope of the present disclosure, in some embodiments the bin times are selected in the range of about 1 microsecond to about 5 ms. In some embodiments, the bin time is more than about 1, 2, 3, 4, 5, 6, 7, 8, 9, 10, 15, 20, 30, 40, 50, 60, 70, 80, 90, 100, 200, 250, 300, 400, 500, 600, 700, 750, 800, 900, 1000, 2000, 3000, 4000, or 5000 microseconds. In some embodiments, the bin time is less than about 2, 3, 4, 5, 6, 7, 8, 9, 10, 15, 20, 30, 40, 50, 60, 70, 80, 90, 100, 200, 250, 300, 400, 500, 600, 700, 750, 800, 900, 1000, 2000, 3000, 4000, or 5000 microseconds. In some embodiments, the bin time is about 1 to 1000 microseconds. In some embodiments, the bin time is about 1 to 750 microseconds. In some embodiments, the bin time is about 1 to 500 microseconds. In some embodiments, the bin time is about 1 to 250 microseconds. In some embodiments, the bin time is about 1 to 100 microseconds. In some embodiments, the bin time is about 1 to 50 microseconds. In some embodiments, the bin time is about 1 to 40 microseconds. In some embodiments, the bin time is about 1 to 30 microseconds. In some embodiments, the bin time is about 1 to 25 microseconds. In some embodiments, the bin time is about 1 to 20 microseconds. In some embodiments, the bin time is about 1 to 10 microseconds. In some embodiments, the bin time is about 1 to 7.5 microseconds. In some embodiments, the bin time is about 1 to 5 microseconds. In some embodiments, the bin time is about 5 to 500 microseconds. In some embodiments, the bin time is about 5 to 250 microseconds. In some embodiments, the bin time is about 5 to 100 microseconds. In some embodiments, the bin time is about 5 to 50 microseconds. In some embodiments, the bin time is about 5 to 20 microseconds. In some embodiments, the bin time is about 5 to 10 microseconds. In some embodiments, the bin time is about 10 to 500 microseconds. In some embodiments, the bin time is about 10 to 250 microseconds. In some embodiments, the bin time is about 10 to 100 microseconds. In some embodiments, the bin time is about 10 to 50 microseconds. In some embodiments, the bin time is about 10 to 30 microseconds. In some embodiments, the bin time is about 10 to 20 microseconds. In some embodiments, the bin time is about 1 microsecond. In some embodiments, the bin time is about 2 microseconds. In some embodiments, the bin time is about 3 microseconds. In some embodiments, the bin time is about 4 microseconds. In some embodiments, the bin time is about 5 microseconds. In some embodiments, the bin time is about 6 microseconds. In some embodiments, the bin time is about 7 microseconds. In some embodiments, the bin time is about 8 microseconds. In some embodiments, the bin time is about 9 microseconds. In some embodiments, the bin time is about 10 microseconds. In some embodiments, the bin time is about 11 microseconds. In some embodiments, the bin time is about 12 microseconds. In some embodiments, the bin time is about 13 microseconds. In some embodiments, the bin time is about 14 microseconds. In some embodiments, the bin time is about 5 microseconds. In some embodiments, the bin time is about 15 microseconds. In some embodiments, the bin time is about 16 microseconds. In some embodiments, the bin time is about 17 microseconds. In some embodiments, the bin time is about 18 microseconds. In some embodiments, the bin time is about 19 microseconds. In some embodiments, the bin time is about 20 microseconds. In some embodiments, the bin time is about 25 microseconds. In some embodiments, the bin time is about 30 microseconds. In some embodiments, the bin time is about 40 microseconds. In some embodiments, the bin time is about 50 microseconds. In some embodiments, the bin time is about 100 microseconds. In some embodiments, the bin time is about 250 microseconds. In some embodiments, the bin time is about 500 microseconds. In some embodiments, the bin time is about 750 microseconds. In some embodiments, the bin time is about 1000 microseconds.

In some embodiments, determining the concentration of a particle-label complex in a sample includes determining the background noise level. In some embodiments, the background noise level is determined from the mean noise level, or the root-mean-square noise. In other cases, a typical noise value or a statistical value is chosen. In most cases, the noise is expected to follow a Poisson distribution.

Thus, as a label is encountered in the interrogation space, it is irradiated by the laser beam to generate a burst of photons. The photons emitted by the label are discriminated from background light or background noise emission by considering only the bursts of photons that have energy above a predetermined threshold energy level which accounts for the amount of background noise that is present in the sample. Background noise typically includes low frequency emission produced, for example, by the intrinsic fluorescence of non-labeled particles that are present in the sample, the buffer or diluent used in preparing the sample for analysis, Raman scattering and electronic noise. In some embodiments, the value assigned to the background noise is calculated as the average background signal noise detected in a plurality of bins, which are measurements of photon signals that are detected in an interrogation space during a predetermined length of time. Thus in some embodiments, background noise is calculated for each sample as a number specific to that sample.

Given the value for the background noise, the threshold energy level can be assigned. As discussed above, the threshold value is determined to discriminate true signals (due to fluorescence of a label) from the background noise. Care must be taken in choosing a threshold value such that the number of false positive signals from random noise is minimized while the number of true signals which are rejected is also minimized. Methods for choosing a threshold value include determining a fixed value above the noise level and calculating a threshold value based on the distribution of the noise signal. In one embodiment, the threshold is set at a fixed number of standard deviations above the background level. Assuming a Poisson distribution of the noise, using this method one can estimate the number of false positive signals over the time course of the experiment. In some embodiments, the threshold level is calculated as a value of 4 sigma above the background noise. For example, given an average background noise level of 200 photons, the analyzer system establishes a threshold level of 4√200 above the average background/noise level of 200 photons to be 256 photons. Thus, in some embodiments, determining the concentration of a label in a sample includes establishing the threshold level above which photon signals represent the presence of a label. Conversely, photon signals that have an energy level that is not greater than that of the threshold level indicate the absence of a label.

Many bin measurements are taken to determine the concentration of a sample, and the absence or presence of a label is ascertained for each bin measurement. Typically, 60,000 measurements or more can made in one minute (e.g., in embodiments in which the bin size is 1 ms—for smaller bin sizes the number of measurements is correspondingly larger, e.g., 6,000,000 measurements per minute for a bin size of 10 microseconds). Thus, no single measurement is crucial and the method provides for a high margin of error. The bins that are determined not to contain a label (“no” bins) are discounted and only the measurements made in the bins that are determined to contain label (“yes” bins) are accounted in determining the concentration of the label in the processing sample. Discounting measurements made in the “no” bins or bins that are devoid of label increases the signal to noise ratio and the accuracy of the measurements. Thus, in some embodiments, determining the concentration of a label in a sample includes detecting the bin measurements that reflect the presence of a label.

The signal to noise ratio or the sensitivity of the analyzer system can be increased by minimizing the time that background noise is detected during a bin measurement in which a particle-label complex is detected. For example, in a bin measurement lasting 1 millisecond during which one particle-label complex is detected when passing across an interrogation space within 250 microseconds, 750 microseconds of the 1 millisecond are spent detecting background noise emission. The signal to noise ratio can be improved by decreasing the bin time. In some embodiments, the bin time is 1 millisecond. In other embodiments, the bin time is 750, 500, 250 microseconds, 100 microseconds, 50 microseconds, 25 microseconds or 10 microseconds. Other bin times are as described herein.

Other factors that affect measurements are the brightness or dimness of the fluorescent moiety, the flow rate, and the power of the laser. Various combinations of the relevant factors that allow for detection of label will be apparent to those of skill in the art. In some embodiments, the bin time is adjusted without changing the flow rate. It will be appreciated by those of skill in the art that as bin time decreases, laser power output directed at the interrogation space must increase to maintain a constant total energy applied to the interrogation space during the bin time. For example, if bin time is decreased from 1000 microseconds to 250 microseconds, as a first approximation, laser power output must be increased approximately four-fold. These settings allow for the detection of the same number of photons in a 250 μs as the number of photons counted during the 1000 μs given the previous settings, and allow for faster analysis of sample with lower backgrounds and thus greater sensitivity. In addition, flow rates may be adjusted in order to speed processing of sample. These numbers are merely exemplary, and the skilled practitioner can adjust the parameters as necessary to achieve the desired result.

In some embodiments, the interrogation space encompasses the entire cross-section of the sample stream. When the interrogation space encompasses the entire cross-section of the sample stream, only the number of labels counted and the volume passing through a cross-section of the sample stream in a set length of time are needed to calculate the concentration of the label in the processing sample. In some embodiments, the interrogation space can be defined to be smaller than the cross-sectional area of sample stream by, for example, the interrogation space is defined by the size of the spot illuminated by the laser beam. In some embodiments, the interrogation space can be defined by adjusting the apertures of the analyzer and reducing the illuminated volume that is imaged by the objective lens to the detector. In the embodiments when the interrogation space is defined to be smaller than the cross-sectional area of sample stream, the concentration of the label can be determined by interpolation of the signal emitted by the complex from a standard curve that is generated using one or more samples of known standard concentrations. In yet other embodiments, the concentration of the label can be determined by comparing the measured particles to an internal label standard. In embodiments when a diluted sample is analyzed, the dilution factor is accounted in calculating the concentration of the molecule of interest in the starting sample.

As discussed above, when the interrogation space encompasses the entire cross-section of the sample stream, only the number of labels counted passing through a cross-section of the sample stream in a set length of time (bin) and the volume of sample that was interrogated in the bin are needed to calculate the concentration the sample. The total number of labels contained in the “yes” bins is determined and related to the sample volume represented by the total number of bins used in the analysis to determine the concentration of labels in the processing sample. Thus, in one embodiment, determining the concentration of a label in a processing sample includes determining the total number of labels detected “yes” bins and relating the total number of detected labels to the total sample volume that was analyzed. The total sample volume that is analyzed is the sample volume that is passed through the capillary flow cell and across the interrogation space in a specified time interval. Alternatively, the concentration of the label complex in a sample is determined by interpolation of the signal emitted by the label in a number of bins from a standard curve that is generated by determining the signal emitted by labels in the same number of bins by standard samples containing known concentrations of the label.

In some embodiments, the number of individual labels that are detected in a bin is related to the relative concentration of the particle in the processing sample. At relatively low concentrations, for example at concentrations below about 10⁻¹⁶ M the number of labels is proportional to the photon signal that is detected in a bin. Thus, at low concentrations of label the photon signal is provided as a digital signal. At relatively higher concentrations, for example at concentrations greater than about 10⁻¹⁶ M, the proportionality of photon signal to a label is lost as the likelihood of two or more labels crossing the interrogation space at about the same time and being counted as one becomes significant. Thus, in some embodiments, individual particles in a sample of a concentration greater than about 10⁻¹⁶ M are resolved by decreasing the length of time of the bin measurement.

Alternatively, in other embodiments, the total photon signal that is emitted by a plurality of particles that are present in any one bin is detected. These embodiments allow for single molecule detectors of the disclosure wherein the dynamic range is at least 3, 3.5, 4, 4.5, 5.5, 6, 6.5, 7, 7.5, 8, or more than 8 logs.

“Dynamic range,” as that term is used herein, refers to the range of sample concentrations that may be quantitated by the instrument without need for dilution or other treatment to alter the concentration of successive samples of differing concentrations, where concentrations are determined with an accuracy appropriate for the intended use. For example, if a microtiter plate contains a sample of 1 femtomolar concentration for an analyte of interest in one well, a sample of 10,000 femtomolar concentration for an analyte of interest in another well, and a sample of 100 femtomolar concentration for the analyte in a third well, an instrument with a dynamic range of at least 4 logs and a lower limit of quantitation of 1 femtomolar is able to accurately quantitate the concentration of all the samples without the need for further treatment to adjust concentration, e.g., dilution. Accuracy may be determined by standard methods, e.g., using a series of standards of concentrations that span the dynamic range and constructing a standard curve. Standard measures of fit of the resulting standard curve may be used as a measure of accuracy, e.g., an r² greater than about 0.7, 0.75, 0.8, 0.85, 0.9, 0.91, 0.92, 0.93, 0.94, 0.95, 0.96, 0.97, 0.98, or 0.99.

Increased dynamic range is achieved by altering the manner in which data from the detector is analyzed, and/or by the use of an attenuator between the detector and the interrogation space. At the low end of the range, where processing sample is sufficiently dilute that each detection event, i.e., each burst of photons above a threshold level in a bin (the “event photons”), likely represents only one label, the data is analyzed to count detection events as single molecules. Thereby each bin is analyzed as a simple “yes” or “no” for the presence of label, as described above. For a more concentrated processing sample, where the likelihood of two or more labels occupying a single bin becomes significant, the number of event photons in a significant number of bins is found to be substantially greater than the number expected for a single label, e.g., the number of event photons in a significant number of bins corresponds to two-fold, three-fold, or more, than the number of event photons expected for a single label. For these samples, the instrument changes its method of data analysis to one of integrating the total number of event photons for the bins of the processing sample. This total will be proportional to the total number of labels that were in all the bins. For an even more concentrated processing sample, where many labels are present in most bins, background noise becomes an insignificant portion of the total signal from each bin, and the instrument changes its method of data analysis to one of counting total photons per bin (including background). An even further increase in dynamic range can be achieved by the use of an attenuator between the flow cell and the detector, when concentrations are such that the intensity of light reaching the detector would otherwise exceed the capacity of the detector for accurately counting photons, i.e., saturate the detector.

The instrument may include a data analysis system that receives input from the detector and determines the appropriate analysis method for the sample being run, and outputs values based on such analysis. The data analysis system may further output instructions to use or not use an attenuator, if an attenuator is included in the instrument.

By utilizing such methods, the dynamic range of the instrument can be dramatically increased. Thus, in some embodiments, the instrument is capable of measuring concentrations of samples over a dynamic range of more than about 1000 (3 log), 10,000 (4 log), 100,000 (5 log), 350,000 (5.5 log), 1,000,000 (6 log), 3,500,000 (6.5 log), 10,000,000 (7 log), 35,000,000 (7.5 log), or 100,000,000 (8 log). In some embodiments, the instrument is capable of measuring concentrations of samples over a dynamic range of more than about 100,000 (5 log). In some embodiments, the instrument is capable of measuring concentrations of samples over a dynamic range of more than about 1,000,000 (6 log). In some embodiments, the instrument is capable of measuring concentrations of samples over a dynamic range of more than about 10,000,000 (7 log). In some embodiments, the instrument is capable of measuring the concentrations of samples over a dynamic range of from about 1-10 femtomolar to at least about 1000; 10,000; 100,000; 350,000; 1,000,000; 3,500,000; 10,000,000; or 35,000,000 femtomolar. In some embodiments, the instrument is capable of measuring the concentrations of samples over a dynamic range of from about 1-10 femtomolar to at least about 10,000 femtomolar. In some embodiments, the instrument is capable of measuring the concentrations of samples over a dynamic range of from about 1-10 femtomolar to at least about 100,000 femtomolar. In some embodiments, the instrument is capable of measuring the concentrations of samples over a dynamic range of from about 1-10 femtomolar to at least about 1,000,000 femtomolar. In some embodiments, the instrument is capable of measuring the concentrations of samples over a dynamic range of from about 1-10 femtomolar to at least about 10,000,000.

In some embodiments, an analyzer or analyzer system of the disclosure is capable of detecting an analyte, e.g., a biomarker, at a limit of detection of less than 1 nanomolar, or 1 picomolar, or 1 femtomolar, or 1 attomolar, or 1 zeptomolar. In some embodiments, the analyzer or analyzer system is capable of detecting a change in concentration of the analyte, or of multiple analytes, e.g., a biomarker or biomarkers, from one sample to another sample of less than about 0.1%, 1%, 2%, 5%, 10%, 20%, 30%, 40%, 50%, 60%, 70% or 80% when the biomarker is present at a concentration of less than 1 nanomolar, or 1 picomolar, or 1 femtomolar, or 1 attomolar, or 1 zeptomolar, in the samples, and when the size of each of the sample is less than about 100, 50, 40, 30, 20, 10, 5, 2, 1, 0.1, 0.01, 0.001, or 0.0001 μl. In some embodiments, the analyzer or analyzer system is capable of detecting a change in concentration of the analyte from a first sample to a second sample of less than about 20%, when the analyte is present at a concentration of less than about 1 picomolar, and when the size of each of the samples is less than about 50 μl. In some embodiments, the analyzer or analyzer system is capable of detecting a change in concentration of the analyte from a first sample to a second sample of less than about 20%, when the analyte is present at a concentration of less than about 100 femtomolar, and when the size of each of the samples is less than about 50 μl. In some embodiments, the analyzer or analyzer system is capable of detecting a change in concentration of the analyte from a first sample to a second sample of less than about 20%, when the analyte is present at a concentration of less than about 50 femtomolar, and when the size of each of the samples is less than about 50 μl. In some embodiments, the analyzer or analyzer system is capable of detecting a change in concentration of the analyte from a first sample to a second sample of less than about 20%, when the analyte is present at a concentration of less than about 5 femtomolar, and when the size of each of the samples is less than about 50 μl. In some embodiments, the analyzer or analyzer system is capable of detecting a change in concentration of the analyte from a first sample to a second sample of less than about 20%, when the analyte is present at a concentration of less than about 5 femtomolar, and when the size of each of the samples is less than about 5 μl. In some embodiments, the analyzer or analyzer system is capable of detecting a change in concentration of the analyte from a first sample to a second sample of less than about 20%, when the analyte is present at a concentration of less than about 1 femtomolar, and when the size of each of the samples is less than about 5 μl.

The single molecule detectors of the present disclosure are capable of detecting molecules of interest in a highly sensitive manner with a very low coefficient of variation (CV). In some embodiments, the CV is less than about 50%, 40%, 30%, 25%, 20%, 15%, 10%, 5%, or less than about 1%. In some embodiments, the CV is less than about 50%. In some embodiments, the CV is less than about 40%. In some embodiments, the CV is less than about 30%. In some embodiments, the CV is less than about 25%. In some embodiments, the CV is less than about 20%. In some embodiments, the CV is less than about 15%. In some embodiments, the CV is less than about 10%. In some embodiments, the CV is less than about 5%. In some embodiments, the CV is less than about 1%. In some embodiments, the limit of detection (LOD) is less than about 100 pg/ml and the CV is less than about 10%. In some embodiments, the limit of detection (LOD) is less than about 50 pg/ml and the CV is less than about 10%. In some embodiments, the limit of detection (LOD) is less than about 40 pg/ml and the CV is less than about 10%. In some embodiments, the limit of detection (LOD) is less than about 30 pg/ml and the CV is less than about 10%. In some embodiments, the limit of detection (LOD) is less than about 20 pg/ml and the CV is less than about 10%. In some embodiments, the limit of detection (LOD) is less than about 15 pg/ml and the CV is less than about 10%. In some embodiments, the limit of detection (LOD) is less than about 10 pg/ml and the CV is less than about 10%. In some embodiments, the limit of detection (LOD) is less than about 5 pg/ml and the CV is less than about 10%. In some embodiments, the limit of detection (LOD) is less than about 1 pg/ml and the CV is less than about 10%. In some embodiments, the limit of detection (LOD) is less than about 0.05 pg/ml and the CV is less than about 10%. In some embodiments, the limit of detection (LOD) is less than about 0.01 pg/ml and the CV is less than about 10%. In some embodiments, the limit of detection (LOD) is less than about 10 pg/ml and the CV is less than about 50%. In some embodiments, the limit of detection (LOD) is less than about 10 pg/ml and the CV is less than about 25%. In some embodiments, the limit of detection (LOD) is less than about 10 pg/ml and the CV is less than about 10%. In some embodiments, the limit of detection (LOD) is less than about 10 pg/ml and the CV is less than about 5%. In some embodiments, the limit of detection (LOD) is less than about 10 pg/ml and the CV is less than about 1%. In some embodiments, the limit of detection (LOD) is less than about 5 pg/ml and the CV is less than about 100%. In some embodiments, the limit of detection (LOD) is less than about 5 pg/ml and the CV is less than about 50%. In some embodiments, the limit of detection (LOD) is less than about 5 pg/ml and the CV is less than about 25%. In some embodiments, the limit of detection (LOD) is less than about 5 pg/ml and the CV is less than about 10%. In some embodiments, the limit of detection (LOD) is less than about 5 pg/ml and the CV is less than about 5%. In some embodiments, the limit of detection (LOD) is less than about 5 pg/ml and the CV is less than about 1%. In some embodiments, the limit of detection (LOD) is less than about 1 pg/ml and the CV is less than about 100%. In some embodiments, the limit of detection (LOD) is less than about 1 pg/ml and the CV is less than about 50%. In some embodiments, the limit of detection (LOD) is less than about 1 pg/ml and the CV is less than about 25%. In some embodiments, the limit of detection (LOD) is less than about 1 pg/ml and the CV is less than about 10%. In some embodiments, the limit of detection (LOD) is less than about 1 pg/ml and the CV is less than about 5%. In some embodiments, the limit of detection (LOD) is less than about 1 pg/ml and the CV is less than about 1%.

Instruments and Systems for Highly Sensitive Analysis

The methods of the disclosure utilize analytical instruments of high sensitivity, e.g., single molecule detectors. Such single molecule detectors include embodiments as hereinafter described.

In some embodiments, the disclosure provides an analyzer system kit for detecting a single protein molecule in a sample, said system includes an analyzer system for detecting a single protein molecule in a sample and least one label that includes a fluorescent moiety and a binding partner for the protein molecule, where the analyzer includes an electromagnetic radiation source for stimulating the fluorescent moiety; a capillary flow cell for passing the label; a source of motive force for moving the label in the capillary flow cell; an interrogation space defined within the capillary flow cell for receiving electromagnetic radiation emitted from the electromagnetic source; and an electromagnetic radiation detector operably connected to the interrogation space for measuring an electromagnetic characteristic of the stimulated fluorescent moiety, where the fluorescent moiety is capable of emitting at least about 200 photons when simulated by a laser emitting light at the excitation wavelength of the moiety, where the laser is focused on a spot not less than about 5 microns in diameter that contains the moiety, and where the total energy directed at the spot by the laser is no more than about 3 microJoules.

In some embodiments of the analyzer system kit, the analyzer includes not more than one interrogation space. In some embodiments, the electromagnetic radiation source is a laser that has a power output of at least about 3, 5, 10, or 20 mW. In some embodiments, the fluorescent moiety includes a fluorescent molecule. In some embodiments, the fluorescent molecule is a dye molecule, such as a dye molecule that includes at least one substituted indolium ring system in which the substituent on the 3-carbon of the indolium ring contains a chemically reactive group or a conjugated substance. In some embodiments, the fluorescent moiety is a quantum dot. In some embodiments, the electromagnetic radiation source is a continuous wave electromagnetic radiation source, such as a light-emitting diode or a continuous wave laser. In some embodiments, the motive force is pressure. In some embodiments, the detector is an avalanche photodiode detector. In some embodiments, the analyzer utilizes a confocal optical arrangement for deflecting a laser beam onto said interrogation space and for imaging said stimulated dye molecule wherein said confocal optical arrangement includes an objective lens having a numerical aperture of at least about 0.8. In some embodiments, the analyzer further includes a sampling system capable of automatically sampling a plurality of samples and providing a fluid communication between a sample container and said interrogation space. In some embodiments, the analyzer system further includes a sample recovery system in fluid communication with said interrogation space, wherein said recovery system is capable of recovering substantially all of said sample. In some embodiments, the kit further includes instructions for use of the system.

In one aspect, the methods described herein utilize an analyzer system capable of detecting a single molecule in a sample. In one embodiment, the analyzer system is capable of single molecule detection of a fluorescently labeled particle wherein the analyzer system detects energy emitted by an excited fluorescent label in response to exposure by an electromagnetic radiation source when the single particle is present in an interrogation space defined within a capillary flow cell fluidly connected to the sampling system of the analyzer system. In a further embodiment of the analyzer system, the single particle moves through the interrogation space of the capillary flow cell by means of a motive force. In another embodiment of the analyzer system, an automatic sampling system may be included in the analyzer system for introducing the sample into the analyzer system. In another embodiment of the analyzer system, a sample preparation system may be included in the analyzer system for preparing a sample. In a further embodiment, the analyzer system may contain a sample recovery system for recovering at least a portion of the sample after analysis is complete.

In one aspect, the analyzer system consists of an electromagnetic radiation source for exciting a single particle labeled with a fluorescent label. In one embodiment, the electromagnetic radiation source of the analyzer system is a laser. In a further embodiment, the electromagnetic radiation source is a continuous wave laser.

In a typical embodiment, the electromagnetic radiation source excites a fluorescent moiety attached to a label as the label passes through the interrogation space of the capillary flow cell. In some embodiments, the fluorescent label moiety includes one or more fluorescent dye molecules. In some embodiments, the fluorescent label moiety is a quantum dot. Any fluorescent moiety as described herein may be used in the label.

A label is exposed to electromagnetic radiation when the label passes through an interrogation space located within the capillary flow cell. The interrogation space is typically fluidly connected to a sampling system. In some embodiments the label passes through the interrogation space of the capillary flow cell due to a motive force to advance the label through the analyzer system. The interrogation space is positioned such that it receives electromagnetic radiation emitted from the radiation source. In some embodiments, the sampling system is an automated sampling system capable of sampling a plurality of samples without intervention from a human operator.

The label passes through the interrogation space and emits a detectable amount of energy when excited by the electromagnetic radiation source. In one embodiment, an electromagnetic radiation detector is operably connected to the interrogation space. The electromagnetic radiation detector is capable of detecting the energy emitted by the label, e.g., by the fluorescent moiety of the label.

In a further embodiment of the analyzer system, the system further includes a sample preparation mechanism where a sample may be partially or completely prepared for analysis by the analyzer system. In some embodiments of the analyzer system, the sample is discarded after it is analyzed by the system. In other embodiments, the analyzer system further includes a sample recovery mechanism whereby at least a portion, or alternatively all or substantially all, of the sample may be recovered after analysis. In such an embodiment, the sample can be returned to the origin of the sample. In some embodiments, the sample can be returned to microtiter wells on a sample microtiter plate. The analyzer system typically further consists of a data acquisition system for collecting and reporting the detected signal.

Single Particle Analyzer

The analyzer system includes an electromagnetic radiation source, a mirror, a lens, a capillary flow cell, a microscopic objective lens, an aperture, a detector lens, a detector filter, a single photon detector, and a processor operatively connected to the detector.

In operation the electromagnetic radiation source is aligned so that its output is reflected off of a front surface of the mirror. The lens focuses the beam onto a single interrogation space in the capillary flow cell. The microscope objective lens collects light from sample particles and forms images of the beam onto the aperture. The aperture affects the fraction of light emitted by the specimen in the interrogation space of the capillary flow cell that can be collected. The detector lens collects the light passing through the aperture and focuses the light onto an active area of the detector after it passes through the detector filters. The detector filters minimize aberrant noise signals due to light scatter or ambient light while maximizing the signal emitted by the excited fluorescent moiety bound to the particle. The processor processes the light signal from the particle according to the methods described herein.

In one embodiment, the microscope objective lens is a high numerical aperture microscope objective. As used herein, “high numerical aperture lens” include a lens with a numerical aperture of equal to or greater than 0.6. The numerical aperture is a measure of the number of highly diffracted image-forming light rays captured by the objective. A higher numerical aperture allows increasingly oblique rays to enter the objective lens and thereby produce a more highly resolved image. Additionally, the brightness of an image increases with a higher numerical aperture. High numerical aperture lenses are commercially available from a variety of vendors, and any one lens having a numerical aperture of equal to or greater than approximately 0.6 may be used in the analyzer system. In some embodiments, the lens has a numerical aperture of about 0.6 to about 1.3. In some embodiments, the lens has a numerical aperture of about 0.6 to about 1.0. In some embodiments, the lens has a numerical aperture of about 0.7 to about 1.2. In some embodiments, the lens has a numerical aperture of about 0.7 to about 1.0. In some embodiments, the lens has a numerical aperture of about 0.7 to about 0.9. In some embodiments, the lens has a numerical aperture of about 0.8 to about 1.3. In some embodiments, the lens has a numerical aperture of about 0.8 to about 1.2. In some embodiments, the lens has a numerical aperture of about 0.8 to about 1.0. In some embodiments, the lens has a numerical aperture of at least about 0.6. In some embodiments, the lens has a numerical aperture of at least about 0.7. In some embodiments, the lens has a numerical aperture of at least about 0.8. In some embodiments, the lens has a numerical aperture of at least about 0.9. In some embodiments, the lens has a numerical aperture of at least about 1.0. In some embodiments, the aperture of the microscope objective lens 305 is approximately 1.25. In an embodiment where a microscope objective lens 305 of 0.8 is used, a Nikon 60×/0.8 NA Achromat lens (Nikon, Inc., USA) can be used.

In some embodiments, the electromagnetic radiation source is a laser that emits light in the visible spectrum. In all embodiments, the electromagnetic radiation source is set such that wavelength of the laser is set such that it is of a sufficient wavelength to excite the fluorescent label attached to the particle. In some embodiments, the laser is a continuous wave laser with a wavelength of 639 nm. In other embodiments, the laser is a continuous wave laser with a wavelength of 532 nm. In other embodiments, the laser is a continuous wave laser with a wavelength of 422 nm. In other embodiments, the laser is a continuous wave laser with a wavelength of 405 nm. Any continuous wave laser with a wavelength suitable for exciting a fluorescent moiety as used in the methods and compositions of the disclosure may be used without departing from the scope of the disclosure.

In a single particle analyzer system, as each particle passes through the beam of the electromagnetic radiation source, the particle enters into an excited state. When the particle relaxes from its excited state, a detectable burst of light is emitted. The excitation-emission cycle is repeated many times by each particle in the length of time it takes for it to pass through the beam allowing the analyzer system to detect tens to thousands of photons for each particle as it passes through an interrogation space. Photons emitted by fluorescent particles are registered by the detector with a time delay indicative of the time for the particle label complex to pass through the interrogation space. The photon intensity is recorded by the detector and sampling time is divided into bins, which are uniform, arbitrary, time segments with freely selectable time channel widths. The number of signals contained in each bin evaluated. One or a combination of several statistical analytical methods are employed in order to determine when a particle is present. Such methods include determining the baseline noise of the analyzer system and setting a signal strength for the fluorescent label at a statistical level above baseline noise to eliminate false positive signals from the detector.

The electromagnetic radiation source is focused onto a capillary flow cell of the analyzer system 300 where the capillary flow cell is fluidly connected to the sample system. The beam from the continuous wave electromagnetic radiation source is optically focused to a specified depth within the capillary flow cell. The beam is directed toward the sample-filled capillary flow cell at an angle perpendicular to the capillary flow cell. The beam is operated at a predetermined wavelength that is selected to excite a particular fluorescent label used to label the particle of interest. The size or volume of the interrogation space is determined by the diameter of the beam together with the depth at which the beam is focused. Alternatively, the interrogation space can be determined by running a calibration sample of known concentration through the analyzer system.

When single molecules are detected in the sample concentration, the beam size and the depth of focus required for single molecule detection are set and thereby define the size of the interrogation space. The interrogation space is set such that, with an appropriate sample concentration, only one particle is present in the interrogation space during each time interval over which time observations are made.

It will be appreciated that the detection interrogation volume as defined by the beam is not perfectly spherically shaped, and typically is a “bow-tie” shape. However, for the purposes of definition, “volumes” of interrogation spaces are defined herein as the volume encompassed by a sphere of a diameter equal to the focused spot diameter of the beam. The focused spot of the beam may have various diameters without departing from the scope of the present disclosure. In some embodiments, the diameter of the focused spot of the beam is about 1 to about 5, 10, 15, or 20 microns, or about 5 to about 10, 15, or 20 microns, or about 10 to about 20 microns, or about 10 to about 15 microns. In some embodiments, the diameter of the focused spot of the beam is about 1, 2, 3, 4, 5, 6, 7, 8, 9, 10, 11, 12, 13, 14, 15, 16, 17, 18, 19, or microns. In some embodiments, the diameter of the focused spot of the beam is about 5 microns. In some embodiments, the diameter of the focused spot of the beam is about 10 microns. In some embodiments, the diameter of the focused spot of the beam is about 12 microns. In some embodiments, the diameter of the focused spot of the beam is about 13 microns. In some embodiments, the diameter of the focused spot of the beam is about 14 microns. In some embodiments, the diameter of the focused spot of the beam is about 15 microns. In some embodiments, the diameter of the focused spot of the beam is about 16 microns. In some embodiments, the diameter of the focused spot of the beam is about 17 microns. In some embodiments, the diameter of the focused spot of the beam is about 18 microns. In some embodiments, the diameter of the focused spot of the beam is about 19 microns. In some embodiments, the diameter of the focused spot of the beam is about 20 microns.

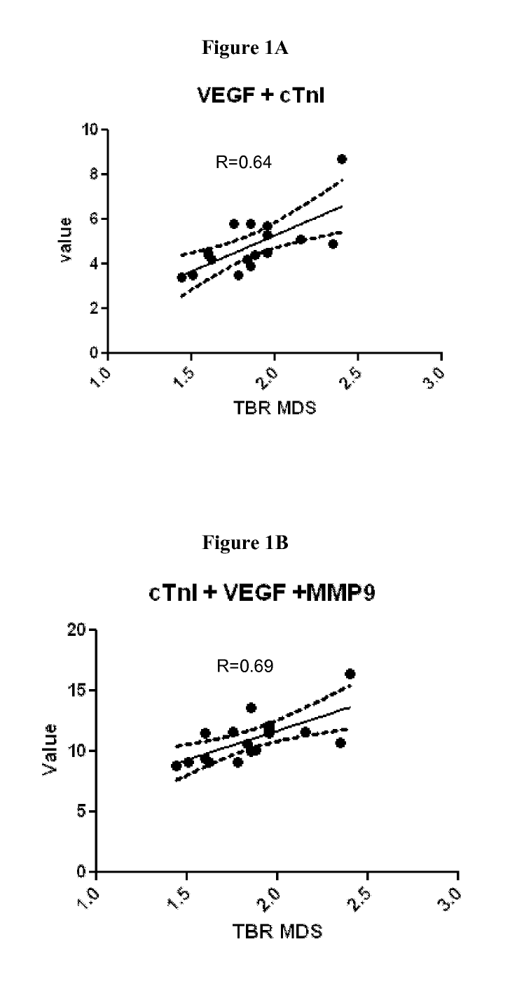

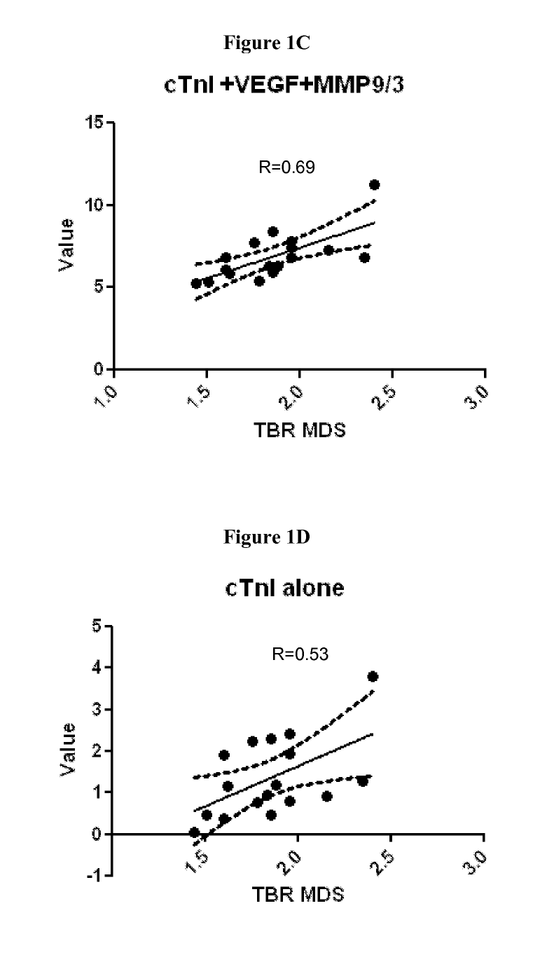

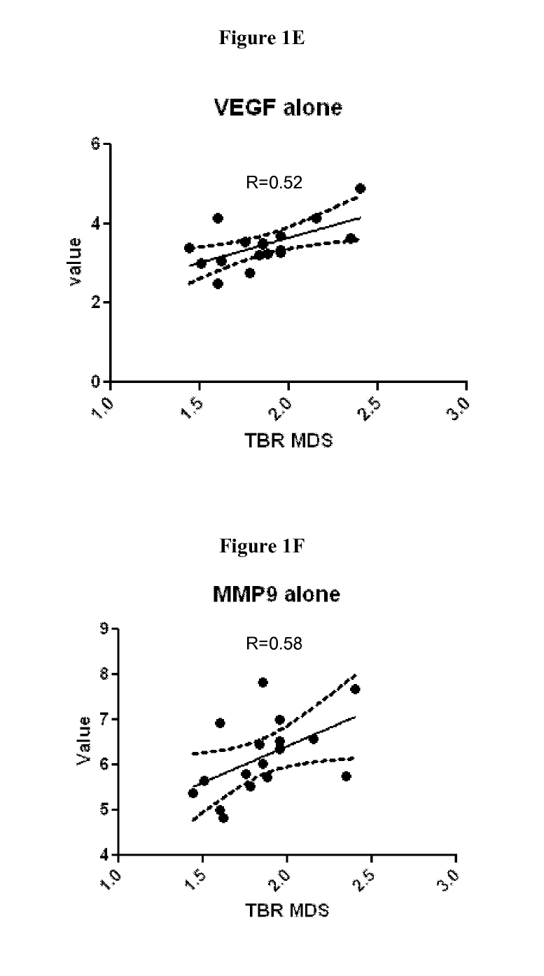

In an alternate embodiment of the single particle analyzer system, more than one electromagnetic radiation source can be used to excite particles labeled with fluorescent labels of different wavelengths. In another alternate embodiment, more than one interrogation space in the capillary flow cell can be used. In another alternate embodiment, multiple detectors can be employed to detect different emission wavelengths from the fluorescent labels. An illustration incorporating each of these alternative embodiments of an analyzer system is shown in FIG. 1B of U.S. Patent Publication No. 2006/0078998.

In some embodiments of the analyzer system, a motive force is required to move a particle through the capillary flow cell of the analyzer system. In one embodiment, the motive force can be a form of pressure. The pressure used to move a particle through the capillary flow cell can be generated by a pump. In some embodiments, a Scivex, Inc. HPLC pump can be used. In some embodiments where a pump is used as a motive force, the sample can pass through the capillary flow cell at a rate of 1 μL/min to about 20 μL/min, or about 5 μL/min to about 20 μL/min. In some embodiments, the sample can pass through the capillary flow cell at a rate of about 5 μL/min. In some embodiments, the sample can pass through the capillary flow cell at a rate of about 10 μL/min. In some embodiments, the sample can pass through the capillary flow cell at a rate of about 15 μL/min. In some embodiments, the sample can pass through the capillary flow cell at a rate of about 20 μL/min. In some embodiments, an electrokinetic force can be used to move the particle through the analyzer system. Such a method has been previously disclosed and is incorporated by reference from previous U.S. Patent Publication No. 2006/0078998.

In one aspect of the analyzer system, the detector of the analyzer system detects the photons emitted by the fluorescent label. In one embodiment, the photon detector is a photodiode. In a further embodiment, the detector is an avalanche photodiode detector. In some embodiments, the photodiodes can be silicon photodiodes with a wavelength detection of 190 nm and 1100 nm. When germanium photodiodes are used, the wavelength of light detected is between 400 nm to 1700 nm. In other embodiments, when an indium gallium arsenide photodiode is used, the wavelength of light detected by the photodiode is between 800 nm and 2600 nm. When lead sulfide photodiodes are used as detectors, the wavelength of light detected is between 1000 nm and 3500 nm.

In some embodiments, the optics of the electromagnetic radiation source and the optics of the detector are arranged in a conventional optical arrangement. In such an arrangement, the electromagnetic radiation source and the detector are aligned on different focal planes. The arrangement of the laser and the detector optics of the analyzer system is that of a conventional optical arrangement.

In some embodiments, the optics of the electromagnetic radiation source and the optics of the detector are arranged in a confocal optical arrangement. In such an arrangement, the electromagnetic radiation source and the detector are aligned on the same focal plane. The confocal arrangement renders the analyzer more robust because the electromagnetic radiation source and the detector optics do not need to be realigned if the analyzer system is moved. This arrangement also makes the use of the analyzer more simplified because it eliminates the need to realign the components of the analyzer system. The beam from an electromagnetic radiation source is focused by the microscope objective to form one interrogation space within the capillary flow cell. A dichroic mirror, which reflects laser light but passes fluorescent light, is used to separate the fluorescent light from the laser light. Filter that is positioned in front of the detector eliminates any non-fluorescent light at the detector. In some embodiments, an analyzer system configured in a confocal arrangement can include two or more interrogations spaces. Such a method has been previously disclosed and is incorporated by reference from previous U.S. Patent Publication No. 2006/0078998.

The laser can be a tunable dye laser, such as a helium-neon laser. The laser can be set to emit a wavelength of 632.8 nm. Alternatively, the wavelength of the laser can be set to emit a wavelength of 543.5 nm or 1523 nm. Alternatively, the electromagnetic laser can be an argon ion laser. In such an embodiment, the argon ion laser can be operated as a continuous gas laser at about 25 different wavelengths in the visible spectrum, the wavelength set between 408.9 and 686.1 nm but at its optimum performance set between 488 and 514.5 nm.

Electromagnetic Radiation Source

In some embodiments of the analyzer system a chemiluminescent label may be used. In such an embodiment, it may not be necessary to utilize an EM source for detection of the particle. In another embodiment, the extrinsic label or intrinsic characteristic of the particle is a light-interacting label or characteristic, such as a fluorescent label or a light-scattering label. In such an embodiment, a source of EM radiation is used to illuminate the label and/or the particle. EM radiation sources for excitation of fluorescent labels are preferred.

In some embodiments, the analyzer system consists of an electromagnetic radiation source. Any number of radiation sources may be used in any one analyzer system 300 without departing from the scope of the disclosure. Multiple sources of electromagnetic radiation have been previously disclosed and are incorporated by reference from previous U.S. Patent Publication No. 2006/0078998. In some embodiments, all the continuous wave electromagnetic (EM) radiation sources emit electromagnetic radiation at the same wavelengths. In other embodiments, different sources emit different wavelengths of EM radiation.

In one embodiment, the EM source(s) are continuous wave lasers producing wavelengths of between 200 nm and 1000 nm. Such EM sources have the advantage of being small, durable and relatively inexpensive. In addition, they generally have the capacity to generate larger fluorescent signals than other light sources. Specific examples of suitable continuous wave EM sources include, but are not limited to: lasers of the argon, krypton, helium-neon, helium-cadmium types, as well as, tunable diode lasers (red to infrared regions), each with the possibility of frequency doubling. The lasers provide continuous illumination with no accessory electronic or mechanical devices, such as shutters, to interrupt their illumination. In an embodiment where a continuous wave laser is used, an electromagnetic radiation source of 3 mW may be of sufficient energy to excite a fluorescent label. A beam from a continuous wave laser of such energy output may be between 2 to 5 μm in diameter. The time of exposure of the particle to laser beam in order to be exposed to 3 mW may be a time period of about 1 msec. In alternate embodiments, the time of exposure to the laser beam may be equal to or less than about 500 μec. In an alternate embodiment, the time of exposure may be equal to or less than about 100 pec. In an alternate embodiment, the time of exposure may be equal to or less than about 50 μec. In an alternate embodiment, the time of exposure may be equal to or less than about 10 pec.

LEDs are another low-cost, high reliability illumination source. Recent advances in ultra-bright LEDs and dyes with high absorption cross-section and quantum yield support the applicability of LEDs to single particle detection. Such lasers could be used alone or in combination with other light sources such as mercury arc lamps, elemental arc lamps, halogen lamps, arc discharges, plasma discharges, light-emitting diodes, or combination of these.

In other embodiments, the EM source could be in the form of a pulse wave laser. In such an embodiment, the pulse size of the laser is an important factor. In such an embodiment, the size, focus spot, and the total energy emitted by the laser is important and must be of sufficient energy as to be able to excite the fluorescent label. When a pulse laser is used, a pulse of longer duration may be required. In some embodiments a laser pulse of 2 nanoseconds may be used. In some embodiments a laser pulse of 5 nanoseconds may be used. In some embodiments a pulse of between 2 to 5 nanoseconds may be used.

The optimal laser intensity depends on the photo bleaching characteristics of the single dyes and the length of time required to traverse the interrogation space (including the speed of the particle, the distance between interrogation spaces if more than one is used and the size of the interrogation space(s)). To obtain a maximal signal, it is desirable to illuminate the sample at the highest intensity which will not result in photo bleaching a high percentage of the dyes. The preferred intensity is one such that no more that 5% of the dyes are bleached by the time the particle has traversed the interrogation space.

The power of the laser is set depending on the type of dye molecules that need to be stimulated and the length of time the dye molecules are stimulated, and/or the speed with which the dye molecules pass through the capillary flow cell. Laser power is defined as the rate at which energy is delivered by the beam and is measured in units of Joules/second, or Watts. It will be appreciated that the greater the power output of the laser, the shorter the time that the laser illuminates the particle may be, while providing a constant amount of energy to the interrogation space while the particle is passing through the space. Thus, in some embodiments, the combination of laser power and time of illumination is such that the total energy received by the interrogation space during the time of illumination is more than about 0.1, 0.5, 1, 2, 3, 4, 5, 6, 7, 8, 9, 10, 12, 15, 20, 25, 30, 35, 40, 45, 50, 60, 70, 80, 90, or 100 microJoule. In some embodiments, the combination of laser power and time of illumination is such that the total energy received by the interrogation space during the time of illumination is less than about 0.5, 1, 2, 3, 4, 5, 6, 7, 8, 9, 10, 12, 15, 20, 25, 30, 35, 40, 45, 50, 60, 70, 80, 90, 100, or 110 microJoule. In some embodiments, the combination of laser power and time of illumination is such that the total energy received by the interrogation space during the time of illumination is between about 0.1 and 100 microJoule. In some embodiments, the combination of laser power and time of illumination is such that the total energy received by the interrogation space during the time of illumination is between about 1 and 100 microJoule. In some embodiments, the combination of laser power and time of illumination is such that the total energy received by the interrogation space during the time of illumination is between about 1 and 50 microJoule. In some embodiments, the combination of laser power and time of illumination is such that the total energy received by the interrogation space during the time of illumination is between about 2 and 50 microJoule. In some embodiments, the combination of laser power and time of illumination is such that the total energy received by the interrogation space during the time of illumination is between about 3 and 60 microJoule. In some embodiments, the combination of laser power and time of illumination is such that the total energy received by the interrogation space during the time of illumination is between about 3 and 50 microJoule. In some embodiments, the combination of laser power and time of illumination is such that the total energy received by the interrogation space during the time of illumination is between about 3 and 40 microJoule. In some embodiments, the combination of laser power and time of illumination is such that the total energy received by the interrogation space during the time of illumination is between about 3 and 30 microJoule. In some embodiments, the combination of laser power and time of illumination is such that the total energy received by the interrogation space during the time of illumination is about 1 microJoule. In some embodiments, the combination of laser power and time of illumination is such that the total energy received by the interrogation space during the time of illumination is about 3 microJoule. In some embodiments, the combination of laser power and time of illumination is such that the total energy received by the interrogation space during the time of illumination is about 5 microJoule. In some embodiments, the combination of laser power and time of illumination is such that the total energy received by the interrogation space during the time of illumination is about 10 microJoule. In some embodiments, the combination of laser power and time of illumination is such that the total energy received by the interrogation space during the time of illumination is about 15 microJoule. In some embodiments, the combination of laser power and time of illumination is such that the total energy received by the interrogation space during the time of illumination is about 20 microJoule. In some embodiments, the combination of laser power and time of illumination is such that the total energy received by the interrogation space during the time of illumination is about 30 microJoule. In some embodiments, the combination of laser power and time of illumination is such that the total energy received by the interrogation space during the time of illumination is about 40 microJoule. In some embodiments, the combination of laser power and time of illumination is such that the total energy received by the interrogation space during the time of illumination is about 50 microJoule. In some embodiments, the combination of laser power and time of illumination is such that the total energy received by the interrogation space during the time of illumination is about 60 microJoule. In some embodiments, the combination of laser power and time of illumination is such that the total energy received by the interrogation space during the time of illumination is about 70 microJoule. In some embodiments, the combination of laser power and time of illumination is such that the total energy received by the interrogation space during the time of illumination is about 80 microJoule. In some embodiments, the combination of laser power and time of illumination is such that the total energy received by the interrogation space during the time of illumination is about 90 microJoule. In some embodiments, the combination of laser power and time of illumination is such that the total energy received by the interrogation space during the time of illumination is about 100 microJoule.

In some embodiments, the laser power output is set to at least about 1 mW, 2 mW, 3 mW, 4 mW, 5 mW, 6, mw, 7 mW, 8 mW, 9 mW, 10 mW, 13 mW, 15 mW, 20 mW, 25 mW, 30 mW, 40 mW, 50 mW, 60 mW, 70 mW, 80 mW, 90 mW, 100 mW, or more than 100 mW. In some embodiments, the laser power output is set to at least about 1 mW. In some embodiments, the laser power output is set to at least about 3 mW. In some embodiments, the laser power output is set to at least about 5 mW. In some embodiments, the laser power output is set to at least about 10 mW. In some embodiments, the laser power output is set to at least about 15 mW. In some embodiments, the laser power output is set to at least about 20 mW. In some embodiments, the laser power output is set to at least about 30 mW. In some embodiments, the laser power output is set to at least about 40 mW. In some embodiments, the laser power output is set to at least about 50 mW. In some embodiments, the laser power output is set to at least about 60 mW. In some embodiments, the laser power output is set to at least about 90 mW.

The time that the laser illuminates the interrogation space can be set to no less than about 1, 2, 3, 4, 5, 10, 15, 20, 30, 40, 50, 60, 70, 80, 90, 100, 150, 200, 150, 300, 350, 400, 450, 500, 600, 700, 800, 900, or 1000 microseconds. The time that the laser illuminates the interrogation space can be set to no more than about 2, 3, 4, 5, 10, 15, 20, 30, 40, 50, 60, 70, 80, 90, 100, 150, 200, 150, 300, 350, 400, 450, 500, 600, 700, 800, 900, 1000, 1500, or 2000 microseconds. The time that the laser illuminates the interrogation space can be set between about 1 and 1000 microseconds. The time that the laser illuminates the interrogation space can be set between about 5 and 500 microseconds. The time that the laser illuminates the interrogation space can be set between about 5 and 100 microseconds. The time that the laser illuminates the interrogation space can be set between about 10 and 100 microseconds. The time that the laser illuminates the interrogation space can be set between about 10 and 50 microseconds. The time that the laser illuminates the interrogation space can be set between about 10 and 20 microseconds. The time that the laser illuminates the interrogation space can be set between about 5 and 50 microseconds. The time that the laser illuminates the interrogation space can be set between about 1 and 100 microseconds. In some embodiments, the time that the laser illuminates the interrogation space is about 1 microsecond. In some embodiments, the time that the laser illuminates the interrogation space is about 5 microseconds. In some embodiments, the time that the laser illuminates the interrogation space is about 10 microseconds. In some embodiments, the time that the laser illuminates the interrogation space is about 25 microseconds. In some embodiments, the time that the laser illuminates the interrogation space is about 50 microseconds. In some embodiments, the time that the laser illuminates the interrogation space is about 100 microseconds. In some embodiments, the time that the laser illuminates the interrogation space is about 250 microseconds. In some embodiments, the time that the laser illuminates the interrogation space is about 500 microseconds. In some embodiments, the time that the laser illuminates the interrogation space is about 1000 microseconds.

For example, the time that the laser illuminates the interrogation space can be set to 1 millisecond, 250 microseconds, 100 microseconds, 50 microseconds, 25 microseconds or 10 microseconds with a laser that provides a power output of 3 mW, 4 mw, 5 mW, or more than 5 mW. In some embodiments, a label is illuminated with a laser that provides a power output of 3 mW and illuminates the label for about 1000 microseconds. In other embodiments, a label is illuminated for less than 1000 milliseconds with a laser providing a power output of not more than about 20 mW. In other embodiments, the label is illuminated with a laser power output of 20 mW for less than or equal to about 250 microseconds. In some embodiments, the label is illuminated with a laser power output of about 5 mW for less than or equal to about 1000 microseconds.

Capillary Flow Cell

The capillary flow cell is fluidly connected to the sample system. In one embodiment, the interrogation space of an analyzer system is determined by the cross sectional area of the corresponding beam and by a segment of the beam within the field of view of the detector. In one embodiment of the analyzer system, the interrogation space has a volume, as defined herein, of between about between about 0.01 and 500 pL, or between about 0.01 pL and 100 pL, or between about 0.01 pL and 10 pL, or between about 0.01 pL and 1 pL, or between about 0.01 pL and 0.5 pL, or between about 0.02 pL and about 300 pL, or between about 0.02 pL and about 50 pL or between about 0.02 pL and about 5 pL or between about 0.02 pL and about 0.5 pL or between about 0.02 pL and about 2 pL, or between about 0.05 pL and about 50 pL, or between about 0.05 pL and about 5 pL, or between about 0.05 pL and about 0.5 pL, or between about 0.05 pL and about 0.2 pL, or between about 0.1 pL and about 25 pL. In some embodiments, the interrogation space has a volume between about 0.01 pL and 10 pL. In some embodiments, the interrogation space has a volume between about 0.01 pL and 1 pL. In some embodiments, the interrogation space has a volume between about 0.02 pL and about 5 pL. In some embodiments, the interrogation space has a volume between about 0.02 pL and about 0.5 pL. In some embodiments, the interrogation space has a volume between about 0.05 pL and about 0.2 pL. In some embodiments, the interrogation space has a volume of about 0.1 pL. Other useful interrogation space volumes are as described herein. It should be understood by one skilled in the art that the interrogation space can be selected for maximum performance of the analyzer. Although very small interrogation spaces have been shown to minimize the background noise, large interrogation spaces have the advantage that low concentration samples can be analyzed in a reasonable amount of time. In embodiments in which two interrogation spaces and are used, volumes such as those described herein for a single interrogation space may be used.

In one embodiment of the present disclosure, the interrogation spaces are large enough to allow for detection of particles at concentrations ranging from about 1000 femtomolar (fM) to about 1 zeptomolar (zM). In one embodiment of the present disclosure, the interrogation spaces are large enough to allow for detection of particles at concentrations ranging from about 1000 fM to about 1 attomolar (aM). In one embodiment of the present disclosure, the interrogation spaces are large enough to allow for detection of particles at concentrations ranging from about 10 fM to about 1 attomolar (aM). In many cases, the large interrogation spaces allow for the detection of particles at concentrations of less than about 1 fM without additional pre-concentration devices or techniques. One skilled in the art will recognize that the most appropriate interrogation space size depends on the brightness of the particles to be detected, the level of background signal, and the concentration of the sample to be analyzed.

The size of the interrogation space can be limited by adjusting the optics of the analyzer. In one embodiment, the diameter of the beam can be adjusted to vary the volume of the interrogation space. In another embodiment, the field of view of the detector can be varied. Thus, the source and the detector can be adjusted so that single particles will be illuminated and detected within the interrogation space. In another embodiment, the width of aperture that determine the field of view of the detector is variable. This configuration allows for altering the interrogation space, in near real time, to compensate for more or less concentrated samples, ensuring a low probability of two or more particles simultaneously being within an interrogation space. Similar alterations for two or more interrogation spaces may performed.

In another embodiment, the interrogation space can be defined through the use of a calibration sample of known concentration that is passed through the capillary flow cell prior to the actual sample being tested. When only one single particle is detected at a time in the calibration sample as the sample is passing through the capillary flow cell, the depth of focus together with the diameter of the beam of the electromagnetic radiation source determines the size of the interrogation space in the capillary flow cell.

Physical constraints to the interrogation spaces can also be provided by a solid wall. In one embodiment, the wall is one or more of the walls of a flow cell when the sample fluid is contained within a capillary. In one embodiment, the cell is made of glass, but other substances transparent to light in the range of about 200 to about 1,000 nm or higher, such as quartz, fused silica, and organic materials such as Teflon, nylon, plastics, such as polyvinylchloride, polystyrene, and polyethylene, or any combination thereof, may be used without departing from the scope of the present disclosure. Although other cross-sectional shapes (e.g., rectangular, cylindrical) may be used without departing from the scope of the present disclosure, in one embodiment the capillary flow cell has a square cross section. In another embodiment, the interrogation space may be defined at least in part by a channel (not shown) etched into a chip (not shown). Similar considerations apply to embodiments in which two interrogation spaces are used.

The interrogation space is bathed in a fluid. In one embodiment, the fluid is aqueous. In other embodiments, the fluid is non-aqueous or a combination of aqueous and non-aqueous fluids. In addition the fluid may contain agents to adjust pH, ionic composition, or sieving agents, such as soluble macroparticles or polymers or gels. It is contemplated that valves or other devices may be present between the interrogation spaces to temporarily disrupt the fluid connection. Interrogation spaces temporarily disrupted are considered to be connected by fluid.

In another embodiment of the disclosure, an interrogation space is the single interrogation space present within the flow cell which is constrained by the size of a laminar flow of the sample material within a diluent volume, also called sheath flow. In these and other embodiments, the interrogation space can be defined by sheath flow alone or in combination with the dimensions of the illumination source or the field of view of the detector. Sheath flow can be configured in numerous ways, including: The sample material is the interior material in a concentric laminar flow, with the diluent volume in the exterior; the diluent volume is on one side of the sample volume; the diluent volume is on two sides of the sample material; the diluent volume is on multiple sides of the sample material, but not enclosing the sample material completely; the diluent volume completely surrounds the sample material; the diluent volume completely surrounds the sample material concentrically; the sample material is the interior material in a discontinuous series of drops and the diluent volume completely surrounds each drop of sample material.

In some embodiments, single molecule detectors of the disclosure include no more than one interrogation space. In some embodiments, multiple interrogation spaces are used. Multiple interrogation spaces have been previously disclosed and are incorporated by reference from U.S. Patent Publication No. 2006/0078998. One skilled in the art will recognize that in some cases the analyzer will contain a plurality of distinct interrogation spaces. In some embodiments, the analyzer contains 2, 3, 4, 5, 6 or more distinct interrogation spaces.

Motive Force

In one embodiment of the analyzer system, the particles are moved through the interrogation space by a motive force. In some embodiments, the motive force for moving particles is pressure. In some embodiments, the pressure is supplied by a pump, and air pressure source, a vacuum source, a centrifuge, or a combination thereof. In some embodiments, the motive force for moving particles is an electrokinetic force. The use of an electrokinetic force as a motive force has been previously disclosed in a prior application and is incorporated by reference from U.S. Patent Publication No. 2006/0078998.

In one embodiment, pressure can be used as a motive force to move particles through the interrogation space of the capillary flow cell. In a further embodiment, pressure is supplied to move the sample by means of a pump. Suitable pumps are known in the art. In one embodiment, pumps manufactured for HPLC applications, such as those made by Scivax, Inc. can be used as a motive force. In other embodiments, pumps manufactured for microfluidics applications can be used when smaller volumes of sample are being pumped. Such pumps are described in U.S. Pat. Nos. 5,094,594, 5,730,187, 6,033,628, and 6,533,553, which discloses devices which can pump fluid volumes in the nanoliter or picoliter range. Preferably all materials within the pump that come into contact with sample are made of highly inert materials, e.g., polyetheretherketone (PEEK), fused silica, or sapphire.

A motive force is necessary to move the sample through the capillary flow cell to push the sample through the interrogation space for analysis. A motive force is also required to push a flushing sample through the capillary flow cell after the sample has been passed through. A motive force is also required to push the sample back out into a sample recovery vessel, when sample recovery is employed. Standard pumps come in a variety of sizes, and the proper size may be chosen to suit the anticipated sample size and flow requirements. In some embodiments, separate pumps are used for sample analysis and for flushing of the system. The analysis pump may have a capacity of approximately 0.000001 mL to approximately 10 mL, or approximately 0.001 mL to approximately 1 mL, or approximately 0.01 mL to approximately 0.2 mL, or approximately 0.005, 0.01, 0.05, 0.1, or 0.5 mL. Flush pumps may be of larger capacity than analysis pumps. Flush pumps may have a volume of about 0.01 mL to about 20 mL, or about 0.1 mL to about 10 mL, or about 0.1 mL to about 2 mL, or about or about 0.05, 0.1, 0.5, 1, 5, or 10 mL. These pump sizes are illustrative only, and those of skill in the art will appreciate that the pump size may be chosen according to the application, sample size, viscosity of fluid to be pumped, tubing dimensions, rate of flow, temperature, and other factors well known in the art. In some embodiments, pumps of the system are driven by stepper motors, which are easy to control very accurately with a microprocessor.

In preferred embodiments, the flush and analysis pumps are used in series, with special check valves to control the direction of flow. The plumbing is designed so that when the analysis pump draws up the maximum sample, the sample does not reach the pump itself. This is accomplished by choosing the ID and length of the tubing between the analysis pump and the analysis capillary such that the tubing volume is greater than the stroke volume of the analysis pump.

Detectors

In one embodiment, light (e.g., light in the ultra-violet, visible or infrared range) emitted by a fluorescent label after exposure to electromagnetic radiation is detected. The detector or detectors is capable of capturing the amplitude and duration of photon bursts from a fluorescent moiety, and further converting the amplitude and duration of the photon burst to electrical signals. Detection devices such as CCD cameras, video input module cameras, and Streak cameras can be used to produce images with contiguous signals. In another embodiment, devices such as a bolometer, a photodiode, a photodiode array, avalanche photodiodes, and photomultipliers which produce sequential signals may be used. Any combination of the aforementioned detectors may also be used. In one embodiment, avalanche photodiodes are used for detecting photons.

Using specific optics between an interrogation space and its corresponding detector, several distinct characteristics of the emitted electromagnetic radiation can be detected including: emission wavelength, emission intensity, burst size, burst duration, and fluorescence polarization. In some embodiments, the detector is a photodiode that is used in reverse bias. A photodiode set in reverse bias usually has an extremely high resistance. This resistance is reduced when light of an appropriate frequency shines on the P/N junction. Hence, a reverse biased diode can be used as a detector by monitoring the current running through it. Circuits based on this effect are more sensitive to light than ones based on zero bias.

In one embodiment of the analyzer system, the photodiode can be an avalanche photodiode, which can be operated with much higher reverse bias than conventional photodiodes, thus allowing each photo-generated carrier to be multiplied by avalanche breakdown, resulting in internal gain within the photodiode, which increases the effective responsiveness (sensitivity) of the device. The choice of photodiode is determined by the energy or emission wavelength emitted by the fluorescently labeled particle. In some embodiments, the photodiode is a silicon photodiode that detects energy in the range of 190-1100 nm; in another embodiment the photodiode is a germanium photodiode that detects energy in the range of 800-1700 nm; in another embodiment the photodiode is an indium gallium arsenide photodiode that detects energy in the range of 800-2600 nm; and in yet other embodiments, the photodiode is a lead sulfide photodiode that detects energy in the range of between less than 1000 nm to 3500 nm. In some embodiments, the avalanche photodiode is a single-photon detector designed to detect energy in the 400 nm to 1100 nm wavelength range. Single photon detectors are commercially available (e.g., Perkin Elmer, Wellesley, Mass.).

In some embodiments, the detector is an avalanche photodiode detector that detects energy between 300 nm and 1700 nm. In one embodiment, silicon avalanche photodiodes can be used to detect wavelengths between 300 nm and 1100 nm. Indium gallium arsenic photodiodes can be used to detect wavelengths between 900 nm and 1700 nm. In some embodiments, an analyzer system can include at least one detector; in other embodiments, the analyzer system can include at least two detectors, and each detector can be chosen and configured to detect light energy at a specific wavelength range. For example, two separate detectors can be used to detect particles that have been tagged with different labels, which upon excitation with an EM source, will emit photons with energy in different spectra. In one embodiment, an analyzer system can include a first detector that can detect fluorescent energy in the range of 450-700 nm such as that emitted by a green dye (e.g., Alexa Fluor 546); and a second detector that can detect fluorescent energy in the range of 620-780 nm such as that emitted by a far-red dye (e.g., Alexa Fluor 647). Detectors for detecting fluorescent energy in the range of 400-600 nm such as that emitted by blue dyes (e.g., Hoechst 33342), and for detecting energy in the range of 560-700 nm such as that emitted by red dyes (Alexa Fluor 546 and Cy3) can also be used.

A system comprising two or more detectors can be used to detect individual particles that are each tagged with two or more labels that emit light in different spectra. For example, two different detectors can detect an antibody that has been tagged with two different dye labels. Alternatively, an analyzer system comprising two detectors can be used to detect particles of different types, each type being tagged with different dye molecules, or with a mixture of two or more dye molecules. For example, two different detectors can be used to detect two different types of antibodies that recognize two different proteins, each type being tagged with a different dye label or with a mixture of two or more dye label molecules. By varying the proportion of the two or more dye label molecules, two or more different particle types can be individually detected using two detectors. It is understood that three or more detectors can be used without departing from the scope of the disclosure.

It should be understood by one skilled in the art that one or more detectors can be configured at each interrogation space, whether one or more interrogation spaces are defined within a flow cell, and that each detector may be configured to detect any of the characteristics of the emitted electromagnetic radiation listed above. The use of multiple detectors, e.g., for multiple interrogation spaces, has been previously disclosed in a prior application and is incorporated by reference here from U.S. Patent Publication No. 2006/0078998. Once a particle is labeled to render it detectable (or if the particle possesses an intrinsic characteristic rendering it detectable), any suitable detection mechanism known in the art may be used without departing from the scope of the present disclosure, for example a CCD camera, a video input module camera, a Streak camera, a bolometer, a photodiode, a photodiode array, avalanche photodiodes, and photomultipliers producing sequential signals, and combinations thereof. Different characteristics of the electromagnetic radiation may be detected including: emission wavelength, emission intensity, burst size, burst duration, fluorescence polarization, and any combination thereof.

Sampling System

In a further embodiment, the analyzer system may include a sampling system to prepare the sample for introduction into the analyzer system. The sampling system included is capable of automatically sampling a plurality of samples and providing a fluid communication between a sample container and a first interrogation space.

In some embodiments, the analyzer system of the disclosure includes a sampling system for introducing an aliquot of a sample into the single particle analyzer for analysis. Any mechanism that can introduce a sample may be used. Samples can be drawn up using either a vacuum suction created by a pump or by pressure applied to the sample that would push liquid into the tube, or by any other mechanism that serves to introduce the sample into the sampling tube. Generally, but not necessarily, the sampling system introduces a sample of known sample volume into the single particle analyzer; in some embodiments where the presence or absence of a particle or particles is detected, precise knowledge of the sample size is not critical. In preferred embodiments the sampling system provides automated sampling for a single sample or a plurality of samples. In embodiments where a sample of known volume is introduced into the system, the sampling system provides a sample for analysis of more than about 0.0001, 0.001, 0.01, 0.1, 1, 2, 5, 10, 20, 30, 40, 50, 60, 70, 80, 90, 100, 150, 200, 500, 1000, 1500, or 2000 μl. In some embodiments the sampling system provides a sample for analysis of less than about 2000, 1000, 500, 200, 100, 90, 80, 70, 60, 50, 40, 30, 20, 10, 5, 2, 1, 0.1, 0.01, or 0.001 μl. In some embodiments the sampling system provides a sample for analysis of between about 0.01 and 1500 μl, or about 0.1 and 1000 μl, or about 1 and 500 μl, or about 1 and 100 μl, or about 1 and 50 μl, or about 1 and 20 μl. In some embodiments, the sampling system provides a sample for analysis between about 5 μl and 200 μl, or about 5 μl and about 100 μl, or about 5 μl and 50 μl. In some embodiments, the sampling system provides a sample for analysis between about 10 μl and 200 μl, or between about 10 μl and 100 μA, or between about 10 μl and 50 μl. In some embodiments, the sampling system provides a sample for analysis between about 0.5 μl and about 50 μl.

Because of the sensitivity of the methods of the present disclosure, very small sample volumes can be used. For example, the methods here can be used to measure VEGF in small sample volumes, e.g., 10 μl or less, compared to the standard sample volume of 100 μl. The present disclosure enables a greater number of samples to provide quantifiable results in small volume samples compared to other methods. For example, a lysate prepared from a typical 1 mm needle biopsy may have a volume less than or equal to 10 μl. Using the present disclosure, such sample can be assayed. In some embodiments, the present disclosure allows the use of sample volume under 100 μl. In some embodiments, the present disclosure allows the use of sample volume under 90 μl. In some embodiments, the present disclosure allows the use of sample volume under 80 μl. In some embodiments, the present disclosure allows the use of sample volume under 70 μl. In some embodiments, the present disclosure allows the use of sample volume under 60 μl. In some embodiments, the present disclosure allows the use of sample volume under 50 μl. In some embodiments, the present disclosure allows the use of sample volume under 40 μl. In some embodiments, the present disclosure allows the use of sample volume under 30 μl. In some embodiments, the present disclosure allows the use of sample volume under 25 μl. In some embodiments, the present disclosure allows the use of sample volume under 20 μl. In some embodiments, the present disclosure allows the use of sample volume under 15 μl. In some embodiments, the present disclosure allows the use of sample volume under 10 μl. In some embodiments, the present disclosure allows the use of sample volume under 5 μl. In some embodiments, the present disclosure allows the use of sample volume under 1 μl. In some embodiments, the present disclosure allows the use of sample volume under 0.05 μl. In some embodiments, the present disclosure allows the use of sample volume under 0.01 μl. In some embodiments, the present disclosure allows the use of sample volume under 0.005 μl. In some embodiments, the present disclosure allows the use of sample volume under 0.001 μl. In some embodiments, the present disclosure allows the use of sample volume under 0.0005 μl. In some embodiments, the present disclosure allows the use of sample volume under 0.0001 μl.

In some embodiments, the sampling system provides a sample size that can be varied from sample to sample. In these embodiments, the sample size may be any one of the sample sizes described herein, and may be changed with every sample, or with sets of samples, as desired.

Sample volume accuracy, and sample to sample volume precision of the sampling system, is required for the analysis at hand. In some embodiments, the precision of the sampling volume is determined by the pumps used, typically represented by a CV of less than about 50, 40, 30, 20, 10, 5, 4, 3, 2, 1, 0.5, 0.1, 0.05, or 0.01% of sample volume. In some embodiments, the sample to sample precision of the sampling system is represented by a CV of less than about 50, 40, 30, 20, 10, 5, 4, 3, 2, 1, 0.5, 0.1, 0.05, or 0.01%. In some embodiments, the intra-assay precision of the sampling system is represented by a CV of less than about 10, 5, 1, 0.5, or 0.1%. In some embodiments, the intra-assay precision of the sampling system shows a CV of less than about 10%. In some embodiments, the interassay precision of the sampling system is represented by a CV of less than about 5%. In some embodiments, the interassay precision of the sampling system shows a CV of less than about 1%. In some embodiments, the interassay precision of the sampling system is represented by a CV of less than about 0.5%. In some embodiments, the interassay precision of the sampling system shows a CV of less than about 0.1%.

In some embodiments, the sampling system provides low sample carryover, advantageous in that an additional wash step is not required between samples. Thus, in some embodiments, sample carryover is less than about 1, 0.5, 0.1, 0.05, 0.04, 0.03, 0.02, 0.01, 0.005, or 0.001%. In some embodiments, sample carryover is less than about 0.02%. In some embodiments, sample carryover is less than about 0.01%.

In some embodiments the sampler provides a sample loop. In these embodiments, multiple samples are drawn into tubing sequentially and each is separated from the others by a “plug” of buffer. The samples typically are read one after the other with no flushing in between. Flushing is done once at the end of the loop. In embodiments where a buffer “plug” is used, the plug may be recovered ejecting the buffer plug into a separate well of a microtiter plate.

The sampling system may be adapted for use with standard assay equipment, for example, a 96-well microtiter plate, or, preferably, a 384-well plate. In some embodiments the system includes a 96 well plate positioner and a mechanism to dip the sample tube into and out of the wells, e.g., a mechanism providing movement along the X, Y, and Z axes. In some embodiments, the sampling system provides multiple sampling tubes from which samples may be stored and extracted from, when testing is commenced. In some embodiments, all samples from the multiple tubes are analyzed on one detector. In other embodiments, multiple single molecule detectors may be connected to the sample tubes. Samples may be prepared by steps that include operations performed on sample in the wells of the plate prior to sampling by the sampling system, or sample may be prepared within the analyzer system, or some combination of both.

Sample Preparation System

Sample preparation includes the steps necessary to prepare a raw sample for analysis. These steps can involve, by way of example, one or more steps of: separation steps such as centrifugation, filtration, distillation, chromatography; concentration, cell lysis, alteration of pH, addition of buffer, addition of diluents, addition of reagents, heating or cooling, addition of label, binding of label, cross-linking with illumination, separation of unbound label, inactivation and/or removal of interfering compounds and any other steps necessary for the sample to be prepared for analysis by the single particle analyzer. In some embodiments, blood is treated to separate out plasma or serum. Additional labeling, removal of unbound label, and/or dilution steps may also be performed on the serum or plasma sample.

In some embodiments, the analyzer system includes a sample preparation system that performs some or all of the processes needed to provide a sample ready for analysis by the single particle analyzer. This system may perform any or all of the steps listed above for sample preparation. In some embodiments samples are partially processed by the sample preparation system of the analyzer system. Thus, in some embodiments, a sample may be partially processed outside the analyzer system first. For example, the sample may be centrifuged first. The sample may then be partially processed inside the analyzer by a sample preparation system. Processing inside the analyzer includes labeling the sample, mixing the sample with a buffer and other processing steps that will be known to one in the art. In some embodiments, a blood sample is processed outside the analyzer system to provide a serum or plasma sample, which is introduced into the analyzer system and further processed by a sample preparation system to label the particle or particles of interest and, optionally, to remove unbound label. In other embodiments preparation of the sample can include immunodepletion of the sample to remove particles that are not of interest or to remove particles that can interfere with sample analysis. In yet other embodiments, the sample can be depleted of particles that can interfere with the analysis of the sample. For example, sample preparation can include the depletion of heterophilic antibodies, which are known to interfere with immunoassays that use non-human antibodies to directly or indirectly detect a particle of interest. Similarly, other proteins that interfere with measurements of the particles of interest can be removed from the sample using antibodies that recognize the interfering proteins.

In some embodiments, the sample can be subjected to solid phase extraction prior to being assayed and analyzed. For example, a serum sample that is assayed for cAMP can first be subjected to solid phase extraction using a c18 column to which it binds. Other proteins such as proteases, lipases and phosphatases are washed from the column, and the cAMP is eluted essentially free of proteins that can degrade or interfere with measurements of cAMP. Solid phase extraction can be used to remove the basic matrix of a sample, which can diminish the sensitivity of the assay. In yet other embodiments, the particles of interest present in a sample may be concentrated by drying or lyophilizing a sample and solubilizing the particles in a smaller volume than that of the original sample.

In some embodiments the analyzer system provides a sample preparation system that provides complete preparation of the sample to be analyzed on the system, such as complete preparation of a blood sample, a saliva sample, a urine sample, a cerebrospinal fluid sample, a lymph sample, a BAL sample, a biopsy sample, a forensic sample, a bioterrorism sample, and the like. In some embodiments the analyzer system provides a sample preparation system that provides some or all of the sample preparation. In some embodiments, the initial sample is a blood sample that is further processed by the analyzer system. In some embodiments, the sample is a serum or plasma sample that is further processed by the analyzer system. The serum or plasma sample may be further processed by, e.g., contacting with a label that binds to a particle or particles of interest; the sample may then be used with or without removal of unbound label.

In some embodiments, sample preparation is performed, either outside the analysis system or in the sample preparation component of the analysis system, on one or more microtiter plates, such as a 96-well plate. Reservoirs of reagents, buffers, and the like can be in intermittent fluid communication with the wells of the plate by means of tubing or other appropriate structures, as are well-known in the art. Samples may be prepared separately in 96 well plates or tubes. Sample isolation, label binding and, if necessary, label separation steps may be done on one plate. In some embodiments, prepared particles are then released from the plate and samples are moved into tubes for sampling into the sample analysis system. In some embodiments, all steps of the preparation of the sample are done on one plate and the analysis system acquires sample directly from the plate. Although this embodiment is described in terms of a 96-well plate, it will be appreciated that any vessel for containing one or more samples and suitable for preparation of sample may be used. For example, standard microtiter plates of 384 or 1536 wells may be used. More generally, in some embodiments, the sample preparation system is capable of holding and preparing more than about 5, 10, 20, 30, 40, 50, 60, 70, 80, 90, 100, 200, 300, 500, 1000, 5000, or 10,000 samples. In some embodiments, multiple samples may be sampled for analysis in multiple analyzer systems. Thus, in some embodiments, 2 samples, or more than about 2, 3, 4, 5, 7, 10, 15 20, 50, or 100 samples are sampled from the sample preparation system and run in parallel on multiple sample analyzer systems.

Microfluidics systems may also be used for sample preparation and as sample preparation systems that are part of analyzer systems, especially for samples suspected of containing concentrations of particles high enough that detection requires smaller samples. Principles and techniques of microfluidic manipulation are known in the art. See, e.g., U.S. Pat. Nos. 4,979,824; 5,770,029; 5,755,942; 5,746,901; 5,681,751; 5,658,413; 5,653,939; 5,653,859; 5,645,702; 5,605,662; 5,571,410; 5,543,838; 5,480,614; 5,716,825; 5,603,351; 5,858,195; 5,863,801; 5,955,028; 5,989,402; 6,041,515; 6,071,478; 6,355,420; 6,495,104; 6,386,219; 6,606,609; 6,802,342; 6,749,734; 6,623,613; 6,554,744; 6,361,671; 6,143,152; 6,132,580; 5,274,240; 6,689,323; 6,783,992; 6,537,437; 6,599,436; 6,811,668 and published PCT Patent Application No. WO 99/55461(A1). Samples may be prepared in series or in parallel, for use in a single or multiple analyzer systems.

In some embodiments, the sample includes a buffer. The buffer may be mixed with the sample outside the analyzer system, or it may be provided by the sample preparation mechanism. While any suitable buffer can be used, the preferable buffer has low fluorescence background, is inert to the detectably labeled particle, can maintain the working pH and, in embodiments wherein the motive force is electrokinetic, has suitable ionic strength for electrophoresis. The buffer concentration can be any suitable concentration, such as in the range from about 1 to about 200 mM. Any buffer system may be used as long as it provides for solubility, function, and delectability of the molecules of interest. In some embodiments, e.g., for application using pumping, the buffer is selected from the group consisting of phosphate, glycine, acetate, citrate, acidulate, carbonate/bicarbonate, imidazole, triethanolamine, glycine amide, borate, MES, Bis-Tris, ADA, aces, PIPES, MOPSO, Bis-Tris Propane, BES, MOPS, TES, HEPES, DIPSO, MOBS, TAPSO, Trizma, HEPPSO, POPSO, TEA, EPPS, Tricine, Gly-Gly, Bicine, HEPBS, TAPS, AMPD, TABS, AMPSO, CHES, CAPSO, AMP, CAPS, and CABS. The buffer can also be selected from the group consisting of Gly-Gly, bicine, tricine, 2-morpholine ethanesulfonic acid (MES), 4-morpholine propanesulfonic acid (MOPS) and 2-amino-2-methyl-1-propanol hydrochloride (AMP). A useful buffer is 2 mM Tris/borate at pH 8.1, but Tris/glycine and Tris/HCl are also acceptable. Other buffers are as described herein.

Buffers useful for electrophoresis are disclosed in a prior application and are incorporated by reference herein from U.S. Patent Publication No. 2006/0078998.

Sample Recovery

One highly useful feature of embodiments of the analyzers and analysis systems of the disclosure is that the sample can be analyzed without consuming it. This can be especially important when sample materials are limited. Recovering the sample also allows one to do other analyses or reanalyze it. The advantages of this feature for applications where sample size is limited and/or where the ability to reanalyze the sample is desirable, e.g., forensic, drug screening, and clinical diagnostic applications, will be apparent to those of skill in the art.

Thus, in some embodiments, the analyzer system of the disclosure further provides a sample recovery system for sample recovery after analysis. In these embodiments, the system includes mechanisms and methods by which the sample is drawn into the analyzer, analyzed and then returned, e.g., by the same path, to the sample holder, e.g., the sample tube. Because no sample is destroyed and because it does not enter any of the valves or other tubing, it remains uncontaminated. In addition, because all the materials in the sample path are highly inert, e.g., PEEK, fused silica, or sapphire, there is little contamination from the sample path. The use of the stepper motor controlled pumps (particularly the analysis pump) allows precise control of the volumes drawn up and pushed back out. This allows complete or nearly complete recovery of the sample with little if any dilution by the flush buffer. Thus, in some embodiments, more than about 50%, 60%, 70%, 80%, 85%, 90%, 95%, 96%, 97%, 98%, 99%, 99.5%, or 99.9% of the sample is recovered after analysis. In some embodiments, the recovered sample is undiluted. In some embodiments, the recovered sample is diluted less than about 1.5-fold, 1.4-fold, 1.3-fold, 1.2-fold, 1.1-fold, 1.05-fold, 1.01-fold, 1.005-fold, or 1.001-fold.

For sampling and/or sample recovery, any mechanism for transporting a liquid sample from a sample vessel to the analyzer may be used. In some embodiments the inlet end of the analysis capillary has attached a short length of tubing, e.g., PEEK tubing that can be dipped into a sample container, e.g., a test tube or sample well, or can be held above a waste container. When flushing, to clean the previous sample from the apparatus, this tube is positioned above the waste container to catch the flush waste. When drawing a sample in, the tube is put into the sample well or test tube. Typically the sample is drawn in quickly, and then pushed out slowly while observing particles within the sample. Alternatively, in some embodiments, the sample is drawn in slowly during at least part of the draw-in cycle; the sample may be analyzed while being slowly drawn in. This can be followed by a quick return of the sample and a quick flush. In some embodiments, the sample may be analyzed both on the inward (draw-in) and outward (pull out) cycle, which improves counting statistics, e.g., of small and dilute samples, as well as confirming results, and the like. If it is desired to save the sample, it can be pushed back out into the same sample well it came from, or to another. If saving the sample is not desired, the tubing is positioned over the waste container.

Methods Using Highly Sensitive Analysis

The systems, system kits, and methods of the present disclosure make possible measurement of molecules in samples at concentrations far lower than previously measured. The high sensitivity of the instruments, kits, and methods of the disclosure allows the establishment of markers, e.g., biological markers, that have not previously been possible because of a lack of sensitivity of detection. The disclosure also includes the use of the compositions and methods described herein for the discovery of new markers.

There are numerous markers currently available which, while potentially of use in determining a biological state, are not currently of practical use because their lower ranges are unknown. In some cases, abnormally high levels of the marker are detectable by current methodologies, but normal ranges have not been established. In some cases, upper normal ranges of the marker are detectable, but not lower normal ranges, or levels below normal. In some cases, for example, markers specific to tumors, or markers of infection, any level of the marker indicates the potential presence of the biological state, and enhancing sensitivity of detection is an advantage for early diagnosis. In some cases, the rate of change, or lack of change, in the concentration of the marker over multiple timepoints provides the most useful information, but present methods of analysis do not permit determination of levels of the marker at timepoint sampling in the early stages of a condition, when it is typically at its most treatable. In many cases, the marker may be detected at clinically useful levels only through the use of cumbersome methods that are not practical or useful in a clinical setting, such as methods that require complex sample treatment and time-consuming analysis.

In addition, there are potential markers of biological states that exist in sufficiently low concentrations that their presence remains extremely difficult or impossible to detect by current methods.

The analytical methods and compositions of the present disclosure provide levels of sensitivity and precision that allow the detection of markers for biological states at concentrations at which the markers have been previously undetectable, thus allowing the “repurposing” of such markers from confirmatory markers, or markers useful only in limited research settings, to diagnostic, prognostic, treatment-directing, or other types of markers useful in clinical settings and/or in large-scale clinical settings such as clinical trials. Such methods allow, e.g., the determination of normal and abnormal ranges for such markers.

The markers thus repurposed can be used for, e.g., detection of normal state (normal ranges), detection of responder/non-responder (e.g., to a treatment, such as administration of a drug); early disease or pathological occurrence detection, disease staging; disease monitoring (monitoring for recurrence of cancer after treatment); study of disease mechanism; and study of treatment toxicity, such as toxicity of drug treatments.

The disclosure thus provides methods and compositions for the sensitive detection of markers, and further methods of establishing values for normal and abnormal levels of the markers. In further embodiments, the disclosure provides methods of diagnosis, prognosis, and/or treatment selection based on values established for the markers. The disclosure also provides compositions for use in such methods, e.g., detection reagents for the ultrasensitive detection of markers.

In some embodiments, the disclosure provides a method of establishing a marker for a biological state, by establishing a range of concentrations for the marker in biological samples obtained from a first population by measuring the concentrations of the marker the biological samples by detecting single molecules of the marker, e.g., by detecting a label that has been attached to a single molecule of the marker. In some embodiments, the marker is a polypeptide or small molecule. The samples may be any sample type described herein, e.g., blood, plasma, serum, or urine.

The method may utilize samples from a first population where the population is a population that does not exhibit the biological state. In the case where the biological state is a disease state, the first population may be a population that does not exhibit the disease, e.g., a “normal” population. In some embodiments the method may further include establishing a range of range of levels for the marker in biological samples obtained from a second population, where the members of the second population exhibit the biological state, by measuring the concentrations of the marker the biological samples by detecting single molecules of the marker. In some embodiments, e.g., cross-sectional studies, the first and second populations are different. In some embodiments, at least one member of the second population is a member of the first population, or at least one member of said the population is a member of the second population. In some embodiments, e.g., longitudinal studies, substantially all the members of the second population are members of the first population who have developed the biological state, e.g., a disease or pathological state.

The detecting of single molecules of the marker is performed using a method as described herein, e.g., a method with a limit of detection for said marker of less than about 1000, 100, 50, 20, 10, 5, 1, 0.5, 0.1, 0.05, 0.01, 0.005, or 0.001 femtomolar of the marker in the samples, by detecting single molecules of the marker. In some embodiments, the limit of detection of said marker is than about 100, 50, 20, 10, 5, 1, 0.5, 0.1, 0.05, 0.01, 0.005, or 0.001 pg/ml of the marker in the samples, by detecting single molecules of the marker.

The biological state may be a phenotypic state; a condition affecting the organism; a state of development; age; health; pathology; disease; disease process; disease staging; infection; toxicity; or response to chemical, environmental, or drug factors (such as drug response phenotyping, drug toxicity phenotyping, or drug effectiveness phenotyping).

In some embodiments, the biological state is a pathological state, including but not limited to inflammation, abnormal cell growth, and abnormal metabolic state. In some embodiments, the state is a disease state. Disease states include, but are not limited to, cancer, cardiovascular disease, inflammatory disease, autoimmune disease, neurological disease, infectious disease and pregnancy related disorders. In some embodiments the state is a disease stage state, e.g., a cancer disease stage state.

The methods may also be used for determination of a treatment response state. In some embodiments, the treatment is a drug treatment. The response may be a therapeutic effect or a side effect, e.g., an adverse effect. Markers for therapeutic effects will be based on the disease or condition treated by the drug. Markers for adverse effects typically will be based on the drug class and specific structure and mechanism of action and metabolism. A common adverse effect is drug toxicity. An example is cardiotoxicity, which can be monitored by the marker cardiac troponin. In some embodiments one or more markers for the disease state and one or more markers for one or more adverse effects of a drug are monitored, typically in a population that is receiving the drug. Samples may be taken at intervals and the respective values of the markers in the samples may be evaluated over time.

The detecting of single molecules of the marker may include labeling the marker with a label comprising a fluorescent moiety capable of emitting at least about 200 photons when simulated by a laser emitting light at the excitation wavelength of the moiety, where the laser is focused on a spot not less than about 5 microns in diameter that contains the moiety, and wherein the total energy directed at the spot by the laser is no more than about 3 microJoules. In some embodiments, the fluorescent moiety includes a molecule that includes at least one substituted indolium ring system in which the substituent on the 3-carbon of the indolium ring contains a chemically reactive group or a conjugated substance. In some embodiments, the fluorescent moiety may include a dye selected from the group consisting of Alexa Fluor 488, Alexa Fluor 532, Alexa Fluor 647, Alexa Fluor 680 or Alexa Fluor 700. In some embodiments, the moiety includes Alexa Fluor 647. In some embodiments, the label further includes a binding partner for the marker, e.g., an antibody specific for said marker, such as a polyclonal antibody or a monoclonal antibody. Binding partners for a variety of markers are described herein.

The method may further include establishing a threshold level for the marker based on the first range, or the first and second ranges, where the presence of marker in a biological sample from an individual at a level above or below the threshold level indicates an increased probability of the presence of the biological state in said individual. An example of a threshold determined for a normal population is the suggested threshold for cardiac troponin of greater than the 99th percentile value in a normal population. Other threshold levels may be determined empirically, i.e., based on data from the first and second populations regarding marker levels and the presence, absence, severity, rate of progression, rate of regression, and the like, of the biological state being monitored. It will be appreciated that threshold levels may be established at either end of a range, e.g., a minimum below which the concentration of the marker in a sample indicates an increased probability of a biological state, and/or a maximum above which the concentration of the marker in a sample indicates an increased probability of a biological state. In some embodiments, a risk stratification may be produced in which two or more ranges of marker concentrations correspond to two or more levels of risk. Other methods of analyzing data from two populations and for markers and producing clinically-relevant values for use by, e.g., physicians and other health care professionals, are well-known in the art.

For some biological markers, the presence of any marker at all is an indication of a disease or pathological state, and the threshold is essentially zero. Other evaluations of marker concentration may also be made, such as in a series of samples, where change in value, rate of change, spikes, decrease, and the like may all provide useful information for determination of a biological state. In addition, panels of markers may be used if it is found that more than one marker provides information regarding a biological state. If panels of markers are used, the markers may be measured separately in separate samples (e.g., aliquots of a common sample) or simultaneously by multiplexing. Examples of panels of markers and multiplexing are given in, e.g., U.S. Patent Publication No. 2006/0078998.

The establishment of such markers and, e.g., reference ranges for normal and/or abnormal states, allow for sensitive and precise determination of the biological state of an organism. Thus, in some embodiments, the disclosure provides a method for detecting the presence or absence of a biological state of an organism, comprising i) measuring the concentration of a marker in a biological sample from the organism, wherein said marker is a marker established through establishing a range of concentrations for said marker in biological samples obtained from a first population by measuring the concentrations of the marker the biological samples by detecting single molecules of the marker; and ii) determining the presence of absence of said biological state based on said concentration of said marker in said organism.

In some embodiments, the disclosure provides a method for detecting the presence or absence of a biological state in an organism, comprising i) measuring the concentrations of a marker in a plurality of biological samples from said organism, wherein said marker is a marker established through establishing a range of concentrations for said marker in biological samples obtained from a first population by measuring the concentrations of the marker the biological samples by detecting single molecules of the marker; and ii) determining the presence of absence of said biological state based on said concentrations of said marker in said plurality of samples. In some embodiments, the samples are of different types, e.g., are samples from different tissue types. In this case, the determining is based on a comparison of the concentrations of said marker in said different types of samples. More commonly, the samples are of the same type, and the samples are taken at intervals. The samples may be any sample type described herein, e.g., blood, plasma, or serum; or urine. Intervals between samples may be minutes, hours, days, weeks, months, or years. In an acute clinical setting, the intervals may be minutes or hours. In settings involving the monitoring of an individual, the intervals may be days, weeks, months, or years.

In many cases, the biological state whose presence or absence is to be detected is a disease phenotype. Thus, in one embodiment, a phenotypic state of interest is a clinically diagnosed disease state. Such disease states include, for example, cancer, cardiovascular disease, inflammatory disease, autoimmune disease, neurological disease, respiratory disease, infectious disease and pregnancy related disorders.

In some embodiments, the method is capable of detecting cTnI at a limit of detection of less than about 100, 80, 60, 50, 30, 20, 10, 5, 1, 0.5, 0.1, 0.05, 0.01, 0.005, 0.001, 0.0005 or 0.0001 pg/ml, e.g., less than about 100 pg/ml. In some embodiments, the method is capable of detecting the cTnI at a limit of detection of less than about 100 pg/ml. In some embodiments, the method is capable of detecting the cTnI a limit of detection of less than about 80 pg/ml. In some embodiments, the method is capable of detecting the cTnI a limit of detection of less than about 60 pg/ml. In some embodiments, the method is capable of detecting the cTnI a limit of detection of less than about 50 pg/ml. In some embodiments, the method is capable of detecting the cTnI a limit of detection of less than about 30 pg/ml. In some embodiments, the method is capable of detecting the cTnI a limit of detection of less than about 25 pg/ml. In some embodiments, the method is capable of detecting the cTnI a limit of detection of less than about 10 pg/ml. In some embodiments, the method is capable of detecting the cTnI a limit of detection of less than about 5 pg/ml. In some embodiments, the method is capable of detecting the cTnI a limit of detection of less than about 1 pg/ml. In some embodiments, the method is capable of detecting the cTnI a limit of detection of less than about 0.5 pg/ml. In some embodiments, the method is capable of detecting the cTnI at a limit of detection of less than about 0.1 pg/ml. In some embodiments, the method is capable of detecting the cTnI at a limit of detection of less than about 0.05 pg/ml. In some embodiments, the method is capable of detecting the cTnI at a limit of detection of less than about 0.01 pg/ml. In some embodiments, the method is capable of detecting the cTnI at a limit of detection of less than about 0.005 pg/ml. In some embodiments, the method is capable of detecting the cTnI at a limit of detection of less than about 0.001 pg/ml. In some embodiments, the method is capable of detecting the cTnI at a limit of detection of less than about 0.0005 pg/ml. In some embodiments, the method is capable of detecting the cTnI at a limit of detection of less than about 0.0001 pg/ml.

In some embodiments, the present disclosure provides methods to quantify normal levels of VEGF, cTnI, and MMP-9 and identify abnormally elevated levels of VEGF, cTnI, and MMP-9 indicative of the presence of vulnerable plaque. This measurement can be augmented by co-measurement of other inflammatory cytokines in healthy individuals, where elevated levels are indicative of inflammation. In some embodiments, because of the role of VEGF, cTnI, and MMP-9 in angiogenesis and artherosclerosis, the disclosure can also be used to quantify abnormally elevated levels of VEGF as indicative of vulnerable plaque in conjunction with elevated levels of cTnI and MMP-9. In some embodiments, the method described can be used to quantify normal levels of VEGF, cTnI, and MMP-9, and identify abnormally elevated levels of VEGF, cTnI, and MMP-9 indicative of the presence of vulnerable plaque in subjects with RA.

In one aspect, the present disclosure provides a method for determining the presence or absence of a single molecule of VEGF, cTnI, and MMP-9 or a fragment or complex thereof in a sample, by i) labeling the molecule, fragment, or complex, if present, with a label; and ii) detecting the presence or absence of the label, where the detection of the presence of the label indicates the presence of the single molecule, fragment, or complex of VEGF in the sample. In some embodiments, the methods of the present disclosure are capable of detecting VEGF, cTnI, and MMP-9 at a limit of detection of less than about 115, 100, 80, 60, 50, 30, 20, 10, 5, 1, 0.5, 0.1, 0.05, 0.01, 0.005, 0.001, 0.0005 or 0.0001 pg/ml.

Detection and Monitoring

The methods of the present disclosure can quantify minute changes in level of a biomarker, e.g., cTnI, MMP-9 and VEGF, over time when longitudinal samples are collected from an individual over a defined period of time. The ability to quantify discreet changes is enabled by the combined sensitivity and precision of measurements made when using the described method.

The methods described herein can be used to monitor levels of biomarkers, e.g., VEGF, MMP-9, cTnI, in healthy individuals, with the ability to detect minute elevations in level of analyte indicative of disease risk or early disease. Such elevations above normal can be quantified over time when regular longitudinal samples are collected from an individual. The ability to monitor discreet changes is enabled by the combined sensitivity and precision of measurements made when using the described method.

The method described can be used to monitor levels of biomarkers, e.g., VEGF, MMP-9, cTnI, in individuals for who elevated levels have been observed, with the ability to detect minute decreases in the level of analyte indicative of a return towards a healthy state. Such decreases can be quantified over time when regular longitudinal samples are collected from an individual, and compared to the healthy range. This information can be used to determine success of a therapeutic intervention or a return to a normal, healthy state. The ability to monitor discreet changes is enabled by the combined sensitivity and precision of measurements according to the present disclosure.

The method described can be used to monitor minute changes in level of analyte, e.g., VEGF, MMP-9, cTnI, over time when longitudinal samples are collected from an individual over a defined period of time. The ability to monitor discreet changes is enabled by the combined sensitivity and precision of measurements according to the present disclosure.

Kits

The disclosure further provides kits. In some embodiments, kits include an analyzer system and a label, as previously described. Kits of the disclosure include one or more compositions useful for the sensitive detection of a molecule, such as a marker, as described herein, in suitable packaging. In some embodiments, kits of the disclosure provide a label, as described herein, together with other components such as instructions, reagents, or other components. In some embodiments, the kit provides the label as separate components, in separate containers, such as an antibody and a fluorescent moiety, for attachment before use by the consumer. In some embodiments kits of the disclosure provide binding partner pairs, e.g., antibody pairs, that are specific for a molecule, e.g., a marker, where at least one of the binding partners is a label for the marker, as described herein. In some embodiments, the binding partners, e.g., antibodies, are provided in separate containers. In some embodiments, the binding partners, e.g., antibodies, are provided in the same container. In some embodiments, one of the binding partners, e.g., antibody, is immobilized on a solid support, e.g., a microtiter plate or a paramagnetic bead. In some of these embodiments, the other binding partner, e.g., antibody, is labeled with a fluorescent moiety as described herein.

Binding partners, e.g., antibodies, solid supports, and fluorescent labels for components of the kits may be any suitable such components as described herein.

The kits may additionally include reagents useful in the methods of the disclosure, e.g., buffers and other reagents used in binding reactions, washes, buffers or other reagents for preconditioning the instrument on which assays will be run, filters for filtering reagents, and elution buffers or other reagents for running samples through the instrument.

Kits may include one or more standards, e.g., standards for use in the assays of the disclosure, such as standards of highly purified, e.g., recombinant, protein markers, or various fragments, complexes, and the like, thereof. Kits may further include instructions.

Data Processing

After collection of the single molecule detection data in the sample, in various embodiments, the data is compared to a reference set of data, acquired under the same experimental conditions, to determine if variations exist in the single molecule detection of the analyte which are characteristic of differences of analytical, diagnostic or prognostic value. In exemplary embodiments, differences between the experimental and reference data are indicative of a disease state, progress of a disease state, stage of a disease state or efficacy of a treatment modality for the disease state. A number of means of performing this comparison are of utility. In an exemplary embodiment, multivariate analysis is used.

Discrimination between data acquired from samples having subtle variations requires the use of robust and sensitive methods of analysis. These methods must model for the nonlinearities that can arise due to various causes as well as account for the day to day drifts in instrument settings. Sample handling errors, noise fringes, baseline shifts, batch to batch variations, the presence of nondiagnostic debris and all other factors that adversely affect discrimination are also preferably adequately accounted for and modeled. Lastly, for a method to prove robust it must distinguish between good and poor quality data, and exclude samples not representative of the reference set. The non-representative samples are referred to as outlier samples. An outlier sample is a sample that is statistically different from all other samples in the reference set. In the case of single molecule detection, an outlier data set can result from samples with less than an optimal number of copies of the single molecule, and/or specimens that are rich in nondiagnostic debris.

In various embodiments, the reference data set is representative of all expected variations in the data set for a selected single molecule. The data of all samples is then processed using methods such as, for example, classical methods of spectroscopic data analysis or multivariate analysis.

Multivariate analysis has been used to analyze biological samples. For example, Robinson, et al. in U.S. Pat. No. 4,975,581 (issued Dec. 4, 1990) describe a quantitative method to determine the similarities of a biological analyte in known biological fluids using multivariate analysis.

Principal Component Analysis (PCA) and discriminate analysis are employed to distinguish between normal and abnormal biological samples. See, Ge, et al., Applied Spectroscopy 49:432-436 (1995). Haaland, et al., in U.S. Pat. No. 5,596,992 (issued Jan. 28, 1997) teach the use of multivariate methods to detect differences between normal and malignant cell samples.

In the present disclosure, when multivariate analysis is used, the comparison of single molecule detection data can be carried out by a partial least squares (PLS) or principal component analysis (PCA) statistical method on data which can be preprocessed (i.e., smoothed and/or derivatized), or unsmoothed and underivatized. Preferably, comparisons using principle component regression (PCR) are carried out using PCA. A number of computer programs are available which carry out these statistical methods, including PCR-32® (from Bio-Rad, Cambridge, Mass., USA) and PLS-PLUS® and DISCRIMINATE® (from Galactic Industries, Salem, N.H., USA). Discussions of the underlying theory and calculations can be found in, for example, Haaland, et al., Anal. Chem. 60:1193-1202 (1988); Cahn, et al., Applied Spectroscopy, 42:865-872 (1988); and Martens, et al., MULTIVARIATE CALIBRATION, John Wiley and Sons, New York, N.Y. (1989). Both PCR and PLS use a library of data derived from single molecules of a reference single molecule sample to create a reference set, wherein each of the data sets are acquired under essentially identical conditions. The data analysis techniques consist of data compression (in the case of PCR, this step is known as PCA), and linear regression. Using a linear combination of factors or principal components, a reconstructed data set is derived. This reconstructed data set is compared with the data set of unknown specimens which serves as the basis for classification.

In certain aspects of the present disclosure the predicted scores generated for individual data sets are “averaged” over the data sets acquired for a particular species of detected single molecule. The “averaged” score from the individual data sets over the sample are “averaged” over the collection of samples. The method of “averaging” can consist of simply taking the arithmetic mean of the predicted scores or can rely on other statistical methods for determining population distributions known in the art. The methods include, for example, determining the median and determining the mean of the predicted score population. The extent to which a population is scattered on either side of the determined center is assessed by establishing a measure of dispersion such as, for example, the standard deviation, the interquartile range, the range and the mean deviation. Other methods, of use in practicing the present disclosure, for establishing both the “average” value for the population of prediction scores and the extent of population scatter will be apparent to those of skill in the art.

Prior to the analysis of unknown samples, another set of data of the same single molecule can be used to validate and optimize the reference. This second set of data enhance the prediction accuracy of the PCR or PLS model by determining the rank of the model. The optimal rank is determined from a range of ranks by comparing the PCR or PLS predictions with known diagnoses. Increasing or decreasing the rank from what was determined optimal can adversely affect the PLS or PCR predictions. For example, as the rank is gradually decreased from optimal to suboptimal, PCR or PLS would account for less and less variations in the reference spectra. In contrast, a gradual increase in the rank beyond what was determined optimal would cause the PCR or PLS methodologies to model random variation rather than significant information in the reference spectra.

Generally, the more single molecule detection data a reference set includes, the better is the model, and the better are the chances to account for batch to batch variations, baseline shifts and the nonlinearities that can arise due to instrument drifts and changes in the refractive index. Errors due to poor sample handling and preparation, sample impurities, and operator mistakes can also be accounted for so long as the reference data render a true representation of the unknown samples.

Another advantage to using PCR and PLS analysis is that these methods measure the data noise level of unknown samples relative to the reference data. Biological samples are subject to numerous sources of perturbations. Some of these perturbations drastically affect the quality of data, and adversely influence the results of a “diagnosis”. Consequently, it is preferable to distinguish between data that conform with the reference data, and those that do not (e.g., the outlier samples). The F-ratio is a powerful tool in detecting conformity or a lack of fit of a data set (sample) to the reference data. In general F-ratios considerably greater than those of the reference indicate “lack of fit” and should be excluded from the analysis. The ability to exclude outlier samples adds to the robustness and reliability of PCR and PLS as it avoids the creation of a “diagnosis” from inferior and corrupted data. F-ratios can be calculated by the methods described in Haaland, et al., Anal. Chem. 60:1193-1202 (1988), and Cahn, et al., Applied Spectroscopy 42:865-872 (1988).

When discriminating between single molecule data from different samples, the biological materials no longer have known concentrations of constituents. As a result, in exemplary embodiments, the reference data determines the range of variation allowed for a sample to be classified as a member of that reference, and should also include preprocessing algorithms to account for diversities in sample make up.

Other data processing algorithms are of use in the present disclosure. For example in one exemplary embodiment of the present disclosure, the markers may be first identified using a statistically weighted difference between control individuals and diseased patients, calculated as D−NσD*σ_{N} where D is the median concentration of a marker in patients diagnosed as having a particular disease, N is the median of the control individuals, (D* is the standard deviation of D and σ_{N} is the standard deviation of N. The larger the magnitude, the greater the statistical difference between the diseased and normal populations.

According to one embodiment of the disclosure, markers resulting in a statistically weighted difference between control individuals and diseased patients of greater than 0.2, 0.5, 1, 1.5, 2, 2.5 or 3 are identified as diagnostically valuable markers.

As will be appreciated by those of skill, the patient him/herself may be the “control”. For example, if the assay is part of monitoring a course of treatment or of disease progression, data acquired from the patient at the start of the course of treatment or upon discovery or diagnosis of the disease can serve as a reference data set for assessing changes in the disease markers due to treatment or disease progression. Other embodiments in which data from the patient providing a sample for an assay of the disclosure serves as a reference data set or baseline data set will be apparent to those of skill in the art.

Another method of statistical analysis of use in the methods of the disclosure for determining the efficacy of particular candidate analytes, such as particular marker(s), for acting as diagnostic marker(s) is Receiver Operating Characteristic (ROC) curve analysis. An ROC curve is a graphical approach to looking at the effect of a cut-off criterion (e.g., a cut-off value for a diagnostic indicator such as an assay signal or the level of an analyte) on the ability of a diagnostic to correctly identify positive and negative samples or subjects. In an exemplary analysis, one axis of the ROC curve is the true positive rate (TPR, the probability that a true positive sample/subject will be correctly identified as positive) or, alternatively, the false negative rate (FNR=1−TPR, the probability that a true positive sample/subject will be incorrectly identified as a negative). The other axis is the true negative rate (TNR, the probability that a true negative sample will be correctly identified as a negative) or, alternatively, the false positive rate (FPR=1−TNR, the probability that a true negative sample will be incorrectly identified as positive). The ROC curve is generated using assay results for a population of samples/subjects by varying the diagnostic cut-off value used to identify samples/subjects as positive or negative and plotting calculated values of TPR (or FNR) and TNR (or FPR) for each cut-off value. The area under the curve (referred to herein as the ROC area) is one indication of the ability of the diagnostic to separate positive and negative samples/subjects.

One of skill in the art of diagnostic assays and statistical analysis of data, given the teaching and guidance provided herein, will be able to select without undue burden appropriate cut-off values, lines, ratios, zones etc. for best meeting the needs (e.g., sensitivity and specificity) for a particular application. A variety of statistical tools, such as, for example, receiver operating characteristic (ROC) curves, are available for evaluating the effect of adjustments to cut-offs on assay performance (e.g., predicted true positive fraction, false positive fraction, true negative fraction and false negative fraction). Alternatively, statistical analysis of patient populations can allow conversion of specific analyte values into probabilities that the patient has or does not have a disease. For background on the selection and analysis of populations of individuals so as to determine reference ranges see Boyd J. C. “Reference Limits in the Clinical Laboratory” in Professional Practice in Clinical Chemistry: A Companion Text; D. R. Dufour Ed., 1999, Washington D.C.: American Assoc. CLIN. CHEM., Chapter 2, pp. 2-1 to 2-7. For background on the selection of decision limits (i.e., cut-offs) or the calculation, from test results, of disease likelihood see Boyd J. C. “Statistical Aids for Test Interpretation” in Professional Practice in Clinical Chemistry: A Companion Text; D. R. Dufour Ed., 1999, Washington D.C.: American Assoc. CLIN. CHEM., Chapter 3, pp. 3-1 to 3-11.

Methods or Prevention and Treatment

In various aspects, the disclosure is directed to a method for treating vulnerable plaque, preventing vulnerable plaque, and/or preventing or slowing the progression of vulnerable plaque. The method includes determining a patient's vulnerable plaque burden according to the methods described herein, and treating the patient to prevent, slow the progression of or ameliorate the burden.

After vulnerable plaque is diagnosed, it is usually recommended that various treatment or prevention measures start immediately. Important, yet often neglected, aspects of treatment involves lifestyle modifications, such as reduction of sodium intake, regulation of fluid consumption, maintaining an appropriate body weight, and exercise.

Also, it is likely that many agents already proven to prevent coronary events achieve their effect by reducing plaque vulnerability. See Waxman, S., et al., New Drugs and Technologies: Detection and Treatment of Vulnerable Plaques and Vulnerable Patients, Circulation 2006; 114:2390-2411. Studies in animals have demonstrated that lipid-lowering diets and treatments convert fatty, presumably vulnerable plaques into fibrotic, presumably less vulnerable forms. Studies in patients in which the ability of lipid-lowering therapy to prevent events was far greater than its measurable effect on the degree of stenosis support such a concept. Although plaque stabilizing therapies are useful, many events continue to occur in patients receiving the best currently available therapy.

A number of newer systemic pharmacological approaches to coronary prevention are related to plaque vulnerability. For example, lowering of LDL, which can be readily achieved by statin therapy, can prevent events resulting from complications from vulnerable plaques. Also, the beneficial effects of statin therapy observed in the coronary circulation have also been observed in the carotid arteries and the aorta.

In addition, it is likely that increases in HDL would increase plaque stability because such changes accelerate reverse cholesterol transport, lowers oxidation level, and decreases inflammation. Clinical studies have documented the prevention of coronary events with HDL raising by niacin and gemfibrozil therapy.

Inhibitors of cholesteryl ester transport protein have emerged as promising agents to increase HDL levels. For example, Torcetrapib and JTT-705 can produce elevations of ≧50% in HDL cholesterol. The atheroprotective effects of apoA-1 Milano, a synthetic form of HDL, have also received considerable attention. Additional approaches in this active field include the use of small molecules to increase HDL levels and a variety of nuclear hormone receptor agonists to increase ABC gene transcription and HDL levels and/or function. There are also efforts to evaluate systemic administration of unilamellar phospholipid vesicles and gene therapy involving HDL-related proteins.

Lipoprotein-associated phospholipase A2 has emerged as a promising novel target for antiinflammatory therapy of atherosclerosis. This lipase circulates in plasma and can enter the vessel wall. Once in the wall, it interacts with macrophages and lymphocytes to intensify local inflammatory processes. A small-molecule inhibitor of lipoprotein-associated phospholipase A₂ is under study.

An agent that can block endothelial cell signaling pathways that facilitate the movement of monocytes into the vessel wall is being studied. AGI-1067 blocks the production of vascular cell adhesion molecule 1 and has antioxidant and lipid-lowering activity. It has been shown to reduce restenosis after PCI and to increase the luminal dimensions of reference segments. A randomized study is underway in 6000 patients to determine whether AGI-1067 can reduce coronary events.

The following table provides a useful classification to assess the likelihood that a given systemic therapy is plaque stabilizing. Therapies are grouped by the strength of the evidence that they prevent coronary events and the plausibility of a mechanism by which they might do so by stabilizing vulnerable plaques.

Classification of Systemic Therapy for Vulnerable Plaque*

| Therapies | Drug |
|---|---|
| Group 1: therapies with | Statins, ACE inhibitors, β-blockers, aspirin |
| biological plausibility and | |
| positive clinical evidence | |
| Group 2: therapies with | Antioxidants, folic acid, antibiotics |
| biological plausibility but | |
| negative clinical evidence | |
| Group 3: therapies with | Angiotensin-receptor blockers, omega-3 fatty |
| biological plausibility but | acids, other antihypertensive agents, |
| conflicting or inconclusive | cyclooxygenase-2 inhibitors, influenza |
| clinical evidence | vaccine, clopidogrel, metalloproteinase |
| | inhibitors |
| Group 4: therapies with | Peroxisome proliferator-activated receptor |
| biological plausibility but | antagonists, cholesterol ester transfer protein |
| no clinical data available | inhibitors |

*Ambrose JA, D'Agate DJ. Classification of systemic therapies for potential stabilization of the vulnerable plaque to prevent acute myocardial infarction. Am J Cardiol. 2005; 95: 379-382

Various examples of the foregoing compounds and others that may be useful for the treatment and prevention of vulnerable plaque include: anticoagulants (e.g., Dalteparin (Fragmin), Danaparoid (Orgaran) Enoxaparin (Lovenox) Heparin (various) Tinzaparin (Innohep) and Warfarin (Coumadin)); antiplatelet agents (e.g., Aspirin, Ticlopidine, Clopidogrel (Plavix®), and Dipyridamole), Angiotensin-Converting Enzyme (ACE) Inhibitors (e.g., Benazepril (Lotensin), Captopril (Capoten), Enalapril (Vasotec), Fosinopril (Monopril), Lisinopril, (Prinivil, Zestril), Moexipril (Univasc), Perindopril (Aceon), Quinapril (Accupril), Ramipril (Altace), Trandolapril (Mavik); Angiotensin II Receptor Blockers (or inhibitors) (e.g., Candesartan (Atacand), Eprosartan (Teveten), Irbesartan (Avapro), Losartan (Cozaar), Telmisartan (Micardis), and Valsartan (Diovan); Beta Blockers (e.g., Acebutolol (Sectral), Atenolol (Tenormin), Betaxolol (Kerlone), Bisoprolol/hydrochlorothiazide (Ziac), Bisoprolol (Zebeta), Carteolol (Cartrol), Metoprolol (Lopressor, Toprol XL), Nadolol (Corgard), Propranolol (Inderal), Sotalol (Betapace), Timolol (Blocadren); Calcium Channel Blockers (e.g., Amlodipine (Norvasc, Lotrel), Bepridil (Vascor), Diltiazem (Cardizem, Tiazac), Felodipine (Plendil), Nifedipine (Adalat, Procardia), Nimodipine, (Nimotop), Nisoldipine (Sular), Verapamil (Calan, Isoptin, Verelan); Diuretics (e.g., Amiloride (Midamor), Bumetanide (Bumex), Chlorothiazide (Diuril), Chlorthalidone (Hygroton), Furosemide (Lasix), Hydro-chlorothiazide (Esidrix, Hydrodiuril), Indapamide (Lozol) and Spironolactone Aldactone); Vasodialators (e.g., Isosorbide dinitrate (Isordil), Nesiritide (Natrecor), Hydralazine (Apresoline), Nitrates, Minoxidil); Digitalis Preparations (Digoxin and Digitoxin, e.g., Lanoxin); and Statins (e.g., statins, resins, nicotinic acid (niacin), gemfibrozil, clofibrate etc.).

The following are provided for exemplification purposes only and are not intended to limit the scope of the disclosure described in broad terms above. All references cited in this disclosure are incorporated herein by reference.

### EXAMPLES

### Example 1

### Quantification of Vulnerable Plaque Burden Based Upon Radiographic Findings (FDG PET/CT)

Nineteen rheumatoid arthritis patients with moderate RA had a mean age of 51.2 years, and a mean BMI of 29.5. Other parameters, including Disease Activity Score (DAS) and current use of biologics, were collected from each patient. Selected clinical characteristics of the RA patient cohort are summarized in Table 1.

TABLE 1
Summary of Clinical Characteristics of RA Patients

| | Mean |
|---|---|
| DAS 28 | 4.6 |
| Biologic Use | 36% |
| Distribution of CV Risk | |
| Factors* | |
| No CV Risk Factors | 47% |
| 1 CV Risk Factor | 36% |
| 2 or more Risk Factors | 17% |
| History of CAD/CVD | 0% |

*includes all Framingham CV risk factors with the exception of age or gender

For each patient, PET imaging of the ascending aorta and carotid arteries were taken. Vascular inflammation was scored by PET Target to Background (TBR) signal for the left and right carotids and the ascending aorta by the same radiologist. Scores for all patients are shown in Table 2.

TABLE 2
Distribution of PET TBR Signal

| | Mean (SD) | Median (IQR) |
|---|---|---|
| Left Carotid Mean TBR | 1.450 (0.174) | 1.440 (0.270) |
| Left Carotid Max TBR | 1.723 (0.221) | 1.740 (0.250) |
| Left Carotid MDS TBR | 1.744 (0.226) | 1.740 (0.240) |
| Right Carotid Mean TBR | 1.501 (0.220) | 1.510 (0.350) |
| Right Carotid Max TBR | 1.824 (0.304) | 1.750 (0.390) |
| Right Carotid MDS TBR | 1.842 (0.306) | 1.850 (0.360) |
| Ascending Aorta Mean TBR | 1.520 (0.404) | 1.460 (0.505) |
| Ascending Aorta Max TBR | 2.082 (0.574) | 2.050 (0.625) |
| Ascending Aorta MDS TBR | 2.214 (0.626) | 2.230 (0.685) |

MDS, Maximal Diseased Segment
TBR, Target-to-Background Ratio

### Example 2

### Determination of Biomarkers

Blood samples of patients identified in Example 1 and healthy volunteers (n=10) were obtained under IRB approval and informed consent. All specimens were collected under protocol, which included noting time of blood collection into serum tubes, separation of serum from cells and storing of resulting serum at −70° C.

Samples were assayed for cTnI, VEGF and MMP-9 using the highly sensitive ERENNA® immunoassay system (Singulex, Inc) as described previously (see Todd et al., Clin Chem 2007 and U.S. Pat. No. 7,838,250). Briefly, frozen samples were thawed, and if necessary clarified by centrifugation or filtering through a 1.2 um filter in a 96 well plate. For cTnI, the sample was added to a well in a 96 well plate, along with sufficient volume of calibrator diluent (3% BSA, Tris pH 8.0, 150 mM NaCl) to create a final volume of 50 ul. 150 ul of paramagnetic microparticles (MPs, MyOne, Invitrogen Dynal AS; approximately 5-10 ug MPs/well), coated with the capture antibody and diluted in assay buffer (1% BSA, Tris-buffered saline, pH 7.4, with 0.5 mL Triton X-100/L, and heterophile/human antimouse antibody-blocking reagents (from Scantibodies Laboratories, used per the manufacturer's recommendations), were added to each well and incubated for about 1 hour. MPs were separated using a magnetic bed (Ambion). Supernatant was removed, MPs were washed once, and then 20 uL of Alexa dye labeled detection antibody (50-500 mg/L diluted in assay buffer) was added and incubated for about 1 hour at 25° C. with shaking. The MPs were again magnetically separated and washed 5 times using Tris-buffered saline with 0.5 mL Triton X-100/L. After removal of residual wash buffer, 10 uL elution buffer (Glycine pH 2.5) was added. This reagent disrupted antibody-analyte interactions and resulted in the release of detection antibody from the MPs. The solution in each 96-well plate was then transferred to a 384-well filter plate (0.2 um, AcroPrep cat. no. 5070, Pall) containing 10 uL 0.5M TRIS buffer (to neutralize glycine) and centrifuged to create a solution in each well of the 384 well plate containing tris-glycine and eluted Alexa dye labelled detection antibody (The MPs were retained in the 96 well plate via magnetic separation using the magnetic bed). The 384 well plate was read in the ERENNA® system to count individual Alexa dye labeled detection antibody molecules from each well. The concentration of cTnI in each sample was determined via interpolation off a standard curve run with the samples.

Analysis of blood samples for VEGF and MMP9 were conducted with the ERENNA® system using the same assay procedure described above. Importantly, all of the assays had limits of quantification that were lower than the concentration of the analytes measured in serum. This ensured that the measurement of analyte was accurate.

All antibodies and analytes used in the assay were obtained from commercial sources as show in Table 3 (Biospecific, Inc, Emeryville Calif., Abcam, PLC, Cambridge, Mass., R&D Systems, Inc., Minneapolis, Minn.).

TABLE 3
Exemplary antibodies used to detect biomarkers in patient samples

| Biomarker | Capture Antibody | Detection Antibody |
|---|---|---|
| cTnI | Biospecific A34650228P | BioSpecific G-129-C |
| VEGF | AbCam ab36424 | AbCam ab17696 |
| total MMP-9 | R&D MAB936 | R& D AF911 |
| proMMP-9 | R&D MAB9111 | R&D AF911 |

The average levels of biomarker concentrations for RA patients and healthy volunteer control patients (n=10) determined with the above antibodies and procedures are shown in Table 4.

TABLE 4
Average plasma biomarker concentrations of
RA patients and healthy volunteer control patients.

| | cTnI | VEGF | pro-MMP-9 | total-MMP-9 |
|---|---|---|---|---|
| | (pg/mL) | (pg/mL) | (ng/mL) | (ng/mL) |
| RA | 6.33 | 37.32 | 44.31 | 651 |
| Patients | | | | |
| Control | 3.17 | 19.61 | 3.26 | 179 |
| Patients | | | | |

### Example 3

### Determination of Vulnerable Plaque

To further evaluate the relationship between biomarker levels and vulnerable plaque burden/carotid vascular inflammation, the PET imaging TBR signal for average carotid maximal disease segments (MDS) were compared with biomarker levels for cTnI, VEGF, MMP-9 and MMP-8. The results of the Spearman correlation analysis are shown Tables 5-9. The Most Diseased Segment (MDS) is defined as the 1.5 cm segment within the index vessel that shows the highest PET/CT activity at baseline, and is calculated as a mean of maximum TBR values derived from three contiguous axial segments.

TABLE 5
cTnI with carotid vascular inflammation

| | Spearman | |
|---|---|---|
| | coefficient | |
| | (r) | p-value |
| Carotids Mean TBR | 0.55 | 0.02* |
| Carotids Max TBR | 0.57 | 0.01* |
| Carotids MDS | 0.54 | 0.02* |
| Left Carotid Mean TBR | 0.56 | 0.01* |
| Left Carotid Max TBR | 0.60 | 0.006* |
| Left Carotid MDS | 0.55 | 0.02* |
| Right Carotid Mean TBR | 0.48 | 0.04* |
| Right Carotid Max TBR | 0.48 | 0.04* |
| Right Carotid MDS | 0.47 | 0.04* |
| Ascending Aorta Mean TBR | 0.1 | 0.6 |
| Ascending Aorta Max TBR | 0.13 | 0.59 |
| Ascending Aorta MDS | 0.14 | 0.54 |

MDS, Maximal Diseased Segment
TBR, Target-to-Background Ratio
*p-value<0.05

TABLE 6
cTnI and carotid vascular inflammation PET results

| | Beta Coefficient | |
|---|---|---|
| | (SD) from linear | |
| | regression | p-value |
| Carotids Mean TBR | 0.0106 (0.0039) | 0.0155* |
| Carotids Max TBR | 0.0149 (0.0053) | 0.0115* |
| Carotids MDS | 0.0143 (0.0055) | 0.0183* |
| Left Carotid Mean TBR | 0.0101 (0.0037) | 0.0136* |
| Left Carotid Max TBR | 0.0143 (0.0046) | 0.0063* |
| Left Carotid MDS | 0.0132 (0.0049) | 0.0151* |
| Right Carotid Mean TBR | 0.0110 (0.0049) | 0.0374* |
| Right Carotid Max TBR | 0.0155 (0.0069) | 0.0372* |
| Right Carotid MDS | 0.0154 (0.0069) | 0.0406* |
| Ascending Aorta Mean TBR | 0.0044 (1.0063) | 0.6377 |
| Ascending Aorta Max TBR | 0.0074 (1.0066) | 0.5810 |
| Ascending Aorta MDS | 0.0088 (1.0067) | 0.5439 |

MDS, Maximal Diseased Segment
TBR, Target-to-Background Ratio
*p-value<0.05

TABLE 7
VEGF and carotid vascular inflammation

| | Spearman | |
|---|---|---|
| | coefficient (r) | p-value |
| Carotids Mean TBR | 0.53 | 0.02* |
| Carotids Max TBR | 0.59 | 0.008* |
| Carotids MDS | 0.56 | 0.013* |
| Left Carotid Mean TBR | 0.59 | 0.008* |
| Left Carotid Max TBR | 0.70 | 0.001* |
| Left Carotid MDS | 0.66 | 0.002* |
| Right Carotid Mean TBR | 0.43 | 0.07** |
| Right Carotid Max TBR | 0.45 | 0.05** |
| Right Carotid MDS | 0.43 | 0.06** |
| Ascending Aorta Mean | 0.06 | 0.77 |
| TBR | | |
| Ascending Aorta Max | 0.08 | 0.71 |
| TBR | | |
| Ascending Aorta MDS | 0.09 | 0.67 |

MDS, Maximal Diseased Segment
TBR, Target-to-Background Ratio
*p-value<0.05;
**p-value<0.1

TABLE 8
VEGF and carotid vascular inflammation PET results

| | Beta Coefficient | |
|---|---|---|
| | (SD) | |
| | from linear | |
| | regression | p-value |
| Carotids Mean TBR | 0.0038 (0.0015) | 0.0192* |
| Carotids Max TBR | 0.0058 (0.0019) | 0.0081* |
| Carotids MDS | 0.0056 (0.0020) | 0.0129* |
| Left Carotid Mean TBR | 0.0040 (0.0013) | 0.0076* |
| Left Carotid Max TBR | 0.0062 (0.0015) | 0.0009* |
| Left Carotid MDS | 0.0059 (0.0016) | 0.0023* |
| Right Carotid Mean TBR | 0.0036 (0.0019) | 0.0695** |
| Right Carotid Max TBR | 0.0054 (0.0026) | 0.0543** |
| Right Carotid MDS | 0.0052 (0.0027) | 0.0646** |
| Ascending Aorta Mean | 0.0010 (1.0024) | 0.7752 |
| TBR | | |
| Ascending Aorta Max TBR | 0.0018 (1.0024) | 0.7137 |
| Ascending Aorta MDS | 0.0022 1.0025) | 0.6749 |

MDS, Maximal Diseased Segment
TBR, Target-to-Background Ratio
*p-value<0.05;
**p-value<0.1

TABLE 9

| | Spearman | |
|---|---|---|
| | correlation (r) | p-value |
| | MMP8 | |
| Left Carotid Mean TBR | 0.63 | 0.004 |
| Left Carotid Max TBR | 0.71 | <0.001 |
| Left Carotid MDS | 0.66 | 0.002 |
| Right Carotid Mean TBR | 0.49 | 0.03 |
| Right Carotid Max TBR | 0.50 | 0.03 |
| Right Carotid MDS | 0.49 | 0.03 |
| | proMMP9 | |
| Left Carotid Mean TBR | 0.58 | 0.009 |
| Left Carotid Max TBR | 0.70 | <0.001 |
| Left Carotid MDS | 0.66 | <0.001 |
| Right Carotid Mean TBR | 0.42 | 0.07 |
| Right Carotid Max TBR | 0.44 | 0.06 |
| Right Carotid MDS | 0.43 | 0.07 |

Table 10 shows the correlation between the maximal diseased segment and biomarker values for RA patients.

TABLE 10
Biomarker correlations with carotid vascular inflammation

| | Spearman | |
|---|---|---|
| Biomarker | coefficient (r) | p-value |
| VEGF | 0.5586 | 0.0129* |
| cTnI | 0.5349 | 0.0183* |
| tMMP-9 | 0.4287 | 0.067** |
| pMMP-9 | 0.493 | 0.03 |
| MMP-8 | 0.603 | 0.006 |

*p-value<0.05;
**p-value<0.1

These findings indicate that elevated levels of cTnI, VEGF or MMP-9, individually or combined, are associated with PET imaging for carotid vascular inflammation. As such, cTnI, VEGF or MMP-9 concentrations in biological samples (plasma, serum or blood) can be measured as a predictor for increased vulnerable plaque burden. This study suggests that RA patients who have a cTnI, VEGF or MMP-9 levels above a certain threshold in their plasma have an increased risk of carotid vascular inflammation, a key indicator of vulnerable plaque burden. These findings show that cTnI, VEGF or MMP-9 are reliable prognostic biomarkers for vulnerable plaque burden.

### Example 4

### Development of Vulnerable Plaque Score

The measurement of differences in the biomarker concentrations, either up- or down-regulated, singly or in combination, in RA patients provides opportunities for improved correlations with TBR. Combining the markers shown in Table 10 provides an improved correlation between marker(s) and disease. See Table 10 and FIGS. 1A-1F.

TABLE 11
Combination of Biomarkers Improve Correlations with TBR

| | Spearman | |
|---|---|---|
| Model | coefficient (r) | p-value |
| TBR MDS | | |
| cTnI + VEGF | 0.64 | 0.006* |
| cTnI + VEGF/2 | 0.59 | 0.011* |
| cTnI + VEGF/4 | 0.58 | 0.015* |
| cTnI + VEGF + MMP-9 | 0.69 | 0.002* |
| cTnI + VEGF + MMP-9/3 | 0.69 | 0.002* |
| hVEGF pg/mL | 0.52 | 0.035* |
| cTnI pg/mL | 0.53 | 0.029* |
| tMMP-9 ng/mL | 0.58 | 0.015* |

MDS, Maximal Diseased Segment
TBR, Target-to-Background Ratio
*p-value<0.05

### Example 5

### Use of Score to Determine Vulnerable Plaque

Blood levels of certain biomarkers were used to determine vulnerable plaque burden. The measurement of differences in these biomarker concentrations in patients when standardized to healthy volunteer control subjects provides opportunities for improved correlations with TBR. Standardizing test biomarker values to an average value from control subjects by dividing each vulnerable plaque sample results by the healthy volunteer control average creates a ratio for each sample. The biomarker ratios can be compared individually or combined with other biomarker ratios with the TBR scores.

For example, Table 12 shows the biomarker values for the healthy control subjects.

TABLE 12
Biomarker values in healthy plasma

| | IL-6 | hVEGF | cTnI | MMP2 | tMMP9 | proMMP9 |
|---|---|---|---|---|---|---|
| Patient ID | pg/mL | pg/mL | pg/mL | ng/mL | ng/mL | ng/mL |
| P11666563 | 2.63 | 12.59 | 2.13 | 51 | 261 | 3.61 |
| P11666564 | 2.91 | 12.06 | 7.40 | 51 | 247 | 2.65 |
| P11666565 | 8.01 | 29.30 | 4.06 | 37 | 201 | 3.78 |
| P11666566 | 2.43 | 15.00 | 8.71 | 54 | 139 | 2.79 |
| P11666567 | 0.64 | 64.90 | 1.47 | 35 | 133 | 5.37 |
| P11666568 | 1.22 | 15.58 | 1.63 | 38 | 179 | 2.33 |
| P11666569 | 9.00 | 10.25 | 1.19 | 28 | 160 | 4.34 |
| P11666570 | 0.91 | 12.43 | 1.37 | 48 | 210 | 2.99 |
| P11666571 | 6.02 | 7.33 | 1.13 | 59 | 134 | 2.15 |
| P11666572 | 5.79 | 16.65 | 2.66 | 47 | 129 | 2.59 |
| Average | 3.96 | 19.61 | 3.17 | 45 | 179 | 3.26 |

Tables 13A and 13B show the original biomarker values from RA patients and the ratios of the biomarker values from the RA patients to healthy control value.

TABLE 13A
Original biomarker values for RA patients

| | IL-6 | hVEGF | cTnI | MMP2 | tMMP9 | proMMP9 |
|---|---|---|---|---|---|---|
| Lab. ID | pg/mL | pg/mL | pg/mL | ng/mL | ng/mL | ng/mL |
| 100698 | 1.6 | 38 | 3.6 | 51 | 316 | 16 |
| 100703 | 4.3 | 64 | 1.5 | 41 | 1025 | 82 |
| 100704 | 6.3 | 16 | 2.2 | 46 | 249 | 11 |
| 100709 | 5.4 | 30 | 1.1 | 53 | 219 | 11 |
| 100710 | 2.6 | 40 | 2.2 | 44 | 1111 | 96 |
| 100712 | 3.3 | 33 | 1.6 | 42 | 414 | 34 |
| 100713 | 6.7 | 25 | 3.3 | 50 | 305 | 8 |
| 100715 | 46.9 | 28 | 7 | 30 | 675 | 77 |
| 100716 | 30.9 | 22 | 3.2 | 39 | 126 | 6 |
| 100718 | 2.1 | 28 | 2.3 | 38 | 305 | 10 |
| 100719 | 3.8 | 35 | 9.6 | 29 | 331 | 18 |
| 100721 | 5.9 | 34 | 10 | 57 | 2483 | 5 |
| 100722 | 35.7 | 24 | 2.6 | 51 | 640 | 2 |
| 100724 | 6.2 | 62 | 2.5 | 39 | 731 | 62 |
| 100725 | 0.9 | 12 | 6.9 | 44 | 148 | 2 |
| 100726 | 5.2 | 132 | 45 | 62 | 2143 | 323 |
| 100727 | 4.5 | 27 | 11.3 | 44 | 581 | 43 |
| 100729 | 3 | 20 | 1.6 | 36 | 285 | 14 |
| 100731 | 3.5 | 39 | 2.7 | 46 | 279 | 22 |

TABLE 13B
Ratios of original biomarker value/avg healthy control

| | IL-6 | hVEGF | cTnI | tMMP9 | MMP2 | proMMP9 |
|---|---|---|---|---|---|---|
| Lab. ID | pg/mL | pg/mL | pg/mL | ng/mL | ng/mL | ng/mL |
| 100698 | 0.4 | 1.9 | 1.1 | 1.8 | 1.1 | 4.9 |
| 100703 | 1.1 | 3.3 | 0.5 | 5.7 | 0.9 | 25.2 |
| 100704 | 1.6 | 0.8 | 0.7 | 1.4 | 1.0 | 3.4 |
| 100709 | 1.4 | 1.5 | 0.3 | 1.2 | 1.2 | 3.4 |
| 100710 | 0.7 | 2.0 | 0.7 | 6.2 | 1.0 | 29.4 |
| 100712 | 0.8 | 1.7 | 0.5 | 2.3 | 0.9 | 10.4 |
| 100713 | 1.7 | 1.3 | 1.0 | 1.7 | 1.1 | 2.5 |
| 100715 | 11.8 | 1.4 | 2.2 | 3.8 | 0.7 | 23.6 |
| 100716 | 7.8 | 1.1 | 1.0 | 0.7 | 0.9 | 1.8 |
| 100718 | 0.5 | 1.4 | 0.7 | 1.7 | 0.8 | 3.1 |
| 100719 | 1.0 | 1.8 | 3.0 | 1.8 | 0.6 | 5.5 |
| 100721 | 1.5 | 1.7 | 3.2 | 13.9 | 1.3 | 1.5 |
| 100722 | 9.0 | 1.2 | 0.8 | 3.6 | 1.1 | 0.6 |
| 100724 | 1.6 | 3.2 | 0.8 | 4.1 | 0.9 | 19.0 |
| 100725 | 0.2 | 0.6 | 2.2 | 0.8 | 1.0 | 0.6 |
| 100726 | 1.3 | 6.7 | 14.2 | 12.0 | 1.4 | 99.1 |
| 100727 | 1.1 | 1.4 | 3.6 | 3.2 | 1.0 | 13.2 |
| 100729 | 0.8 | 1.0 | 0.5 | 1.6 | 0.8 | 4.3 |
| 100731 | 0.9 | 2.0 | 0.9 | 1.6 | 1.0 | 6.7 |

The ratios can be used as score values for each biomarker. Table 14 shows the addition of the rations for the individual biomarkers to create a patient score for vulnerable plaque.

TABLE 14

| | | | | L. | | R. | |
|---|---|---|---|---|---|---|---|
| | | | | Ca- | | Ca- | |
| | | | | rotid | L. | rotid | R. |
| | | cTnI + | cTnI + | Max | Ca- | Max | Ca- |
| | cTnI + | VEGF + | VEGF + | TBR. | rotid | TBR. | rotid |
| ID | VEGF | TMMP9 | TMMP9 | EBW | MDS | EBW | MDS |
| 698 | 3.1 | 4.8 | 8.0 | 1.93 | 2.13 | 2.57 | 2.63 |
| 703 | 3.7 | 9.5 | 28.9 | 1.54 | 1.54 | 1.68 | 1.68 |
| 704 | 1.5 | 2.9 | 4.9 | 1.6 | 1.78 | 1.75 | 1.79 |
| 709 | 1.9 | 3.1 | 5.3 | 1.36 | 1.46 | 1.36 | 1.41 |
| 710 | 2.7 | 8.9 | 32.2 | 1.99 | 2.06 | 1.78 | 1.85 |
| 712 | 2.2 | 4.5 | 12.6 | 1.8 | 1.8 | 1.92 | 1.92 |
| 713 | 2.3 | 4.0 | 4.8 | 1.74 | 1.74 | 1.96 | 1.96 |
| 715 | 3.6 | 7.4 | 27.3 | 1.83 | 1.83 | 2.08 | 2.08 |
| 716 | 2.1 | 2.8 | 4.0 | 1.65 | 1.65 | 1.59 | 1.59 |
| 718 | 2.2 | 3.9 | 5.2 | 1.87 | 1.87 | 1.98 | 1.98 |
| 719 | 4.8 | 6.7 | 10.3 | 1.63 | 1.65 | 1.75 | 1.85 |
| 721 | 4.9 | 18.8 | 6.4 | 1.81 | 1.79 | 1.95 | 1.93 |
| 722 | 2.0 | 5.6 | 2.7 | 1.76 | 1.69 | 2.02 | 2.02 |
| 724 | 4.0 | 8.0 | 23.0 | 2.1 | 2.1 | 2.18 | 2.18 |
| 725 | 2.8 | 3.6 | 3.4 | 1.47 | 1.47 | 1.74 | 1.74 |
| 726 | 20.9 | 32.9 | 120.0 | 2.36 | 2.36 | 2.45 | 2.45 |
| 727 | 4.9 | 8.2 | 18.1 | 1.8 | 1.79 | 2.04 | 2.12 |
| 729 | 1.5 | 3.1 | 5.8 | 1.51 | 1.51 | 1.55 | 1.55 |
| 731 | 2.8 | 4.4 | 9.6 | 1.74 | 1.74 | 1.36 | 1.36 |

In Table 14, the scores can be correlated with MDS. For example, a score of 120 for combined cTnI, VEGF, and TMMP-9 correlates with R carotid max TBR of 2.45.

Tables 15A, 15B and 15C show the correlations between various TBR values and biomarkers values and scores. Examples of correlations include the correlation between the cTnI pg/mL values and R carotid max TBR. Here, the Spearman correlation coefficient across all samples was 0.52 (p=0.024). The correlation between the cTnI score (ratio to healthy volunteers) was 0.49 (p=0.032). When VEGF is added to the cTnI score, the combined score was 0.49 (p=0.033).

TABLE 15A
Biomarker Values

| | | | | | |
|---|---|---|---|---|---|
| IL-6 | hVEGF | cTnI | tMMP9 | MMP2 | proMMP9 |

pvalues, two tailed

| | | | | | | |
|---|---|---|---|---|---|---|
| L. Carotid | 0.957 | 0.016 | 0.111 | 0.002 | 0.624 | 0.052 |
| Max TBR | | | | | | |
| L. Carotid. | 0.856 | 0.028 | 0.179 | 0.010 | 0.585 | 0.032 |
| MDS | | | | | | |
| R. Carotid. | 0.542 | 0.284 | 0.024 | 0.010 | 0.524 | 0.303 |
| Max TBR | | | | | | |
| R. Carotid. | 0.581 | 0.284 | 0.019 | 0.011 | 0.524 | 0.325 |
| MDS | | | | | | |

Spearman correlation coefficients

| | | | | | | |
|---|---|---|---|---|---|---|
| L. Carotid | 0.01 | 0.54 | 0.38 | 0.66 | 0.12 | 0.45 |
| Max TBR | | | | | | |
| L. Carotid. | −0.04 | 0.50 | 0.32 | 0.57 | 0.13 | 0.49 |
| MDS | | | | | | |
| R. Carotid. | 0.15 | 0.26 | 0.52 | 0.57 | 0.16 | 0.25 |
| Max TBR | | | | | | |
| R. Carotid. | 0.14 | 0.26 | 0.53 | 0.57 | 0.16 | 0.24 |
| MDS | | | | | | |

TABLE 15B
Rations to Healthy Volunteers

| | IL-6 | hVEGF | cTnI | tMMP9 | MMP2 | proMMP9 |
|---|---|---|---|---|---|---|
| L. Carotid | 0.966 | 0.019 | 0.129 | 0.001 | 0.591 | 0.052 |
| Max TBR | | | | | | |
| L. Carotid. | 0.826 | 0.032 | 0.199 | 0.008 | 0.617 | 0.032 |
| MDS | | | | | | |
| R. Carotid. | 0.560 | 0.296 | 0.032 | 0.009 | 0.470 | 0.303 |
| Max TBR | | | | | | |
| R. Carotid. | 0.601 | 0.291 | 0.026 | 0.010 | 0.461 | 0.325 |
| MDS | | | | | | |
| L. Carotid | 0.01 | 0.53 | 0.36 | 0.68 | 0.13 | 0.45 |
| Max TBR | | | | | | |
| L. Carotid. | −0.05 | 0.49 | 0.31 | 0.59 | 0.12 | 0.49 |
| MDS | | | | | | |
| R. Carotid. | 0.14 | 0.25 | 0.49 | 0.58 | 0.18 | 0.25 |
| Max TBR | | | | | | |
| R. Carotid. | 0.13 | 0.26 | 0.51 | 0.58 | 0.18 | 0.24 |
| MDS | | | | | | |

TABLE 15C
Addition of Ratios to Create Scores

| | | cTnI + | cTnI + |
|---|---|---|---|
| | cTnI + | VEGF + | VEGF + |
| | VEGF | tMMP9 | pMMP9 |
| L.Carotid Max TBR | 0.033 | 0.006 | 0.022 |
| L.Carotid MDS | 0.068 | 0.025 | 0.020 |
| R.Carotid Max TBR | 0.033 | 0.019 | 0.210 |
| R.Carotid MDS | 0.025 | 0.018 | 0.220 |
| L.Carotid Max TBR | 0.49 | 0.61 | 0.52 |
| L.Carotid MDS | 0.43 | 0.51 | 0.53 |
| R.Carotid Max TBR | 0.49 | 0.53 | 0.30 |
| R.Carotid MDS | 0.51 | 0.54 | 0.29 |

Although various specific embodiments of the present disclosure have been described herein, it is to be understood that the disclosure is not limited to those precise embodiments and that various changes or modifications can be affected therein by one skilled in the art without departing from the scope and spirit of the disclosure.

The examples given above are merely illustrative and are not meant to be an exhaustive list of all possible embodiments, applications or modifications of the disclosure. Thus, various modifications and variations of the described methods and systems of the disclosure will be apparent to those skilled in the art without departing from the scope and spirit of the disclosure. Although the disclosure has been described in connection with specific embodiments, it should be understood that the disclosure as claimed should not be unduly limited to such specific embodiments. Indeed, various modifications of the described modes for carrying out the disclosure which are obvious to those skilled in molecular biology, immunology, chemistry, biochemistry or in the relevant fields are intended to be within the scope of the appended claims.

It is understood that the disclosure is not limited to the particular methodology, protocols, and reagents, etc., described herein, as these may vary as the skilled artisan will recognize. It is also to be understood that the terminology used herein is used for the purpose of describing particular embodiments only, and is not intended to limit the scope of the disclosure.

Any numerical values recited herein include all values from the lower value to the upper value in increments of one unit provided that there is a separation of at least two units between any lower value and any higher value. As an example, if it is stated that the concentration of a component or value of a process variable such as, for example, size, angle size, pressure, time and the like, is, for example, from 1 to 90, specifically from 20 to 80, more specifically from 30 to 70, it is intended that values such as 15 to 85, 22 to 68, 43 to 51, 30 to 32, etc. are expressly enumerated in this specification. For values which are less than one, one unit is considered to be 0.0001, 0.001, 0.01 or 0.1 as appropriate. These are only examples of what is specifically intended and all possible combinations of numerical values between the lowest value and the highest value enumerated are to be considered to be expressly stated in this application in a similar manner.

Particular methods, devices, and materials are described, although any methods and materials similar or equivalent to those described herein can be used in the practice or testing of the disclosure. The disclosures of all references and publications cited herein are expressly incorporated by reference in their entireties to the same extent as if each were incorporated by reference individually.

What is claimed is:

1. A method for determining vulnerable plaque burden in a subject comprising:
measuring an amount of at least one biomarker in a blood, serum or plasma sample from the subject, wherein the at least one biomarker is selected from the group consisting of vascular endothelial growth factor-A (VEGF), cardiac troponin I (cTnI), and matrix metalloproteinase-9 (MMP-9);
comparing the amount of the at least one biomarker in the biological sample from the subject to a threshold concentration representing the concentration of the at least one biomarker in a population of normal healthy control subjects, and
determining vulnerable plaque burden in the subject when the concentration of at least one biomarker in the sample is greater than the threshold concentration.

2. The method of claim 1, wherein the MMP-9 is total MMP-9 (tMMP-9).

3. The method of claim 1, wherein vulnerable plaque burden is determined when the concentrations of at least two biomarkers in the sample are greater than the threshold concentrations.

4. The method of claim 1, wherein vulnerable plaque burden is determined when the concentrations of VEGF, cTnI and MMP-9 in the sample are greater than the threshold concentrations.

5. The method of claim 1, wherein vulnerable plaque burden is symptomatic of cardiovascular disease.

6. A method for determining vulnerable plaque burden in a subject comprising:
measuring the concentration of one or more biomarkers selected from the group consisting of VEGF, cTnI and MMP-9 in a blood, serum or plasma sample from a patient,
standardizing the biomarker concentrations in the sample to threshold concentrations for the biomarkers in a population of normal healthy controls to create a ratio of the concentrations in the sample and the threshold concentrations,
wherein the ratios represent the vulnerable plaque burden in the subject.

7. A method for determining vulnerable plaque burden in a subject comprising:
measuring an amount of at least two biomarkers selected from VEGF, cTnI or MMP-9 in a blood, serum or plasma sample from a patient,
standardizing the biomarker concentrations in the sample to threshold concentrations of the biomarkers in a population of normal healthy controls to create ratios of the concentrations in the test sample and the threshold concentrations,
adding the ratios to create a score representing vulnerable plaque burden in the subject.

8. The method as in claim 7, further comprising normalizing the score representing vulnerable plaque burden in the subject on a scale of 1-100.

9. The method of claim 7, wherein a score of 1 represents the detection of the least amount of vulnerable plaque and 100 represents the detection of the most vulnerable plaque.

10. The method as in any one of claim 1, wherein measuring the amount of VEGF, cTnI or MMP-9 comprises:
contacting the sample with an antibody specific for the VEGF, cTnI and MMP-9, and
determining the amount of specific binding between the antibody and the VEGF, cTnI and MMP-9 in the sample.

11. The method of claim 11, wherein the measuring is conducted with a single molecule counting assay.

12. The method of claim 11, wherein the antibody for VEGF, cTnI, or MMP-9 binds to a portion or derivative of VEGF, cTnI, and MMP-9.
